SEQUENCE LISTING (6 sequences)

SEQ ID NO: 1 - protein, 985 aa, Artificial Sequence
  Synthetic Sequence
MVFQPISEFLLIRNAGMSMYFNKIISFNIISRIVICIFLICGMFMAGASEKYDANAPQQVQPYSVSSSAFENLHPNNEMESSINPFSASDTERNAAIIDRANKEQETEAVNKMISTGARLAASGRASDVAHSMVGDAVNQEIKQWLNRFGTAQVNLNFDKNFSLKESSLDWLAPWYDSASFLFFSQLGIRNKDSRNTLNLGVGIRTLENGWLYGLNTFYDNDLTGHNHRIGLGAEAWTDYLQLAANGYFRLNGWHSSRDFSDYKERPATGGDLRANAYLPALPQLGGKLMYEQYTGERVALFGKDNLQRNPYAVTAGINYTPVPLLTVGVDQRMGKSSKHETQWNLQMNYRLGESFQSQLSPSAVAGTRLLAESRYNLVDRNNNIVLEYQKQQVVKLTLSPATISGLPGQVYQVNAQVQGASAVREIVWSDAELIAAGGTLTPLSTTQFNLVLPPYKRTAQVSRVTDDLTANFYSLSALAVDHQGNRSNSFTLSVTVQQPQLTLTAAVIGDGAPANGKTAITVEFTVADFEGKPLAGQEVVITTNNGALPNKITEKTDANGVARIALTNTTDGVTVVTAEVEGQRQSVDTHFVKGTIAADKSTLAAVPTSIIADGLMASTITLELKDTYGDPQAGANVAFDTTLGNMGVITDHNDGTYSAPLTSTTLGVATVTVKVDGAAFSVPSVTVNFTADPIPDAGRSSFTVSTPDILADGTMSSTLSFVPVDKNGHFISGMQGLSFTQNGVPVSISPITEQPDSYTATVVGNTAGDVTITPQVDTLILSTLQKKISLFPVPTLTGILVNGQNFATDKGFPKTIFKNATFQLQMDNDVANNTQYEWSSSFTPNVSVNDQGQVTITYQTYSEVAVTAKSKKFPSYSVSYRFYPNRWIYDGGTSLVSSLEASRQCQGSDMSAVLESSRATNGTRAPDGTLWGEWGSLTAYSSDWQSGEYWVKKTSTDFETMNMDTGALVQGPAYLAFPLCALAI

SEQ ID NO: 2 - protein, 529 aa, Artificial Sequence
  Synthetic Sequence
MKKIMLVFITLILISLPIAQQTEAKDASAFHKEDLISSMAPPTSPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEINYDPEGNEIVQHKNWSENNKSKLAHFTSSIYLPGNARNINVYAKECTGLAWEWWRTVIDDRNLPLVKNRNISIWGTTLYPKYSNSVDNPIE

SEQ ID NO: 3 - protein, 658 aa, Artificial Sequence
  Synthetic Sequence
MITHGCYTRTRHKHKLKKTLIMLSAGLGLFFYVNQNSFANGENYFKLGSDSKLLTHDSYQNRLFYTLKTGETVADLSKSQDINLSTIWSLNKHLYSSESEMMKAAPGQQIILPLKKLPFEYSALPLLGSAPLVAAGGVAGHTNKLTKMSPDVTKSNMTDDKALNYAAQQAASLGSQLQSRSLNGDYAKDTALGIAGNQASSQLQAWLQHYGTAEVNLQSGNNFDGSSLDFLLPFYDSEKMLAFGQVGARYIDSRFTANLGAGQRFFLPANMLGYNVFIDQDFSGDNTRLGIGGEYWRDYFKSSVNGYFRMSGWHESYNKKDYDERPANGFDIRFNGYLPSYPALGAKLIYEQYYGDNVALFNSDKLQSNPGAATVGVNYTPIPLVTMGIDYRHGTGNENDLLYSMQFRYQFDKSWSQQIEPQYVNELRTLSGSRYDLVQRNNNIILEYKKQDILSLNIPHDINGTEHSTQKIQLIVKSKYGLDRIVWDDSALRSQGGQIQHSGSQSAQDYQAILPAYVQGGSNIYKVTARAYDRNGNSSNNVQLTITVLSNGQVVDQVGVTDFTADKTSAKADNADTITYTATVKKNGVAQANVPVSFNIVSGTATLGANSAKTDANGKATVTLKSSTPGQVVVSAKTAEMTSALNASAVIFFDGATR

SEQ ID NO: 4 - protein, 850 aa, Artificial Sequence
  Synthetic Sequence
MITHGCYTRTRHKHKLKKTLIMLSAGLGLFFYVNQNSFANGENYFKLGSDSKLLTHDSYQNRLFYTLKTGETVADLSKSQDINLSTIWSLNKHLYSSESEMMKAAPGQQIILPLKKLPFEYSALPLLGSAPLVAAGGVAGHTNKLTKMSPDVTKSNMTDDKALNYAAQQAASLGSQLQSRSLNGDYAKDTALGIAGNQASSQLQAWLQHYGTAEVNLQSGNNFDGSSLDFLLPFYDSEKMLAFGQVGARYIDSRFTANLGAGQRFFLPANMLGYNVFIDQDFSGDNTRLGIGGEYWRDYFKSSVNGYFRMSGWHESYNKKDYDERPANGFDIRFNGYLPSYPALGAKLIYEQYYGDNVALFNSDKLQSNPGAATVGVNYTPIPLVTMGIDYRHGTGNENDLLYSMQFRYQFDKSWSQQIEPQYVNELRTLSGSRYDLVQRNNNIILEYKKQDILSLNIPHDINGTEHSTQKIQLIVKSKYGLDRIVWDDSALRSQGGQIQHSGSQSAQDYQAILPAYVQGGSNIYKVTARAYDRNGNSSNNVQLTITVLSNGQVVDQVGVTDFTADKTSAKADNADTITYTATVKKNGVAQANVPVSFNIVSGTATLGANSAKTDANGKATVTLKSSTPGQVVVSAKTAEMTSALNASAVIFFDGATRVPTLTGILVNGQNFATDKGFPKTIFKNATFQLQMDNDVANNTQYEWSSSFTPNVSVNDQGQVTITYQTYSEVAVTAKSKKFPSYSVSYRFYPNRWIYDGGTSLVSSLEASRQCQGSDMSAVLESSRATNGTRAPDGTLWGEWGSLTAYSSDWQSGEYWVKKTSTDFETMNMDTGALVQGPAYLAFPLCALAI

SEQ ID NO: 5 - protein, 311 aa, Artificial Sequence
  Synthetic Sequence
MARKVDLTSCDREPIHIPGSIQPCGCLLACDAQAVRITRITENAGAFFGRETPRVGELLADYFGETEAHALRNALAQSSDPKRPALIFGWRDGLTGRTFDISLHRHDGTSIIEFEPAAAEQADNPLRLTRQIIARTKELKSLEEMAARVPRYLQAMLGYHRVMLYRFADDGSGMVIGEAKRSDLESFLGQHFPASLVPQQARLLYLKNAIRVVSDSRGISSRIVPEHDASGAALDLSFAHLRSISPCHLEFLRNMGVSASMSLSIIIDGTLWGLIICHHYEPRAVPMAQRVAAEMFADFLSLHFTAAHHQR

SEQ ID NO: 6 - protein, 505 aa, Artificial Sequence
  Synthetic Sequence
MDASAFHKEDLISSMAPPTSPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYPNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEINYDPEGNEIVQHKNWSENNKSKLAHFTSSIYLPGNARNINVYAKECTGLAWEWWRTVIDDRNLPLVKNRNISIWGTTLYPKYSNSVDNPIE

(19) United States
(12) Patent Application Publication
(10) Pub. No.: US 2023/0320591 A1
(43) Pub. Date: Oct. 12, 2023

(54) ENGINEERING MICROORGANISMS FOR DIAGNOSTIC IMAGING

(71) Applicant: Microbial Machines, Inc., Woburn, MA (US)

(72) Inventors: Jeffrey WAGNER, Boston, MA (US); Fred MERMELSTEIN, Newton, MA (US); Carl NOVINA, Newton, MA (US); Robert DISTEL, Framingham, MA (US); Steven NEIER, Waltham, MA (US); Barry POLISKY, Boulder, CO (US)

(21) Appl. No.: 18/000,555

(22) PCT Filed: Jun. 1, 2021
(86) PCT No.: PCT/US21/35120

§ 371 Date: Dec. 2, 2022

Related U.S. Application Data
(60) Provisional application No. 63/033,443, filed on Jun. 2, 2020.

(51) Int. Cl. A61B 5/00; C12N 15/70; C12N 1/20

(52) U.S. Cl. CPC A61B 5/0071; C12N 15/70; C12N 1/205; A61B 5/42; C12R 2001/19

(57) ABSTRACT

The present invention relates to, inter alia, engineered bacteria expressing (i) a surface protein, which specifically interacts cell membrane receptors that are exposed to the luminal side of epithelial cells of diseased gastrointestinal tissue and/or epithelial tissue lining the bile duct, pancreatic duct, or common bile duct, etc., and (ii) a detection marker. The engineered bacteria of the present technology are useful for detecting diseased gastrointestinal tissue and/or epithelial tissue lining the bile duct, pancreatic duct, or common bile duct, etc.

### TECHNICAL FIELD

The present invention relates, inter alia, to engineered bacteria and uses thereof.

### BACKGROUND

The gastrointestinal tract (GI tract) takes in food, digests it to extract and absorb energy and nutrients, and expels the remaining waste as feces. Gastrointestinal diseases (GI diseases) are the diseases involving the organs that form the gastrointestinal tract, which include the mouth, esophagus, stomach and small intestine, large intestine and rectum. GI diseases include Barrett's esophagus, inflammatory bowel disease (IBD), irritable bowel syndrome (IBS), Crohn's disease, ulcerative colitis, and precancerous syndromes, and cancer.

The diagnosis of GI diseases starts with symptoms and medical history. Techniques like endoscopy, colonoscopy and computed tomography (CT) scan aid diagnosis by facilitating viewing of the lumen of the GI tract. For example, focal, irregular and asymmetrical gastrointestinal wall thickening on CT scan suggests a malignancy. Segmental or diffuse gastrointestinal wall thickening can indicate an ischemic, inflammatory or infectious disease. The ability to visualize and remove abnormal cells and diseased tissue varies depending on the skills of the surgeon and visibility of the lesions (e.g. polyps or tumors). Certain abnormally growing lesions are flat or small and therefore, not efficiently visualized and removed even by skilled surgeons. Accordingly, new strategies to improve upon the sensitivity of detection are required.

### SUMMARY

Accordingly, in various aspects, the present invention provides compositions and methods that are useful for detecting diseased tissue of the gastrointestinal tract. An aspect of the present invention relates to a method for detecting diseased epithelial tissue. In some embodiments, wherein the diseased epithelial tissue is selected from gastrointestinal tract epithelium and bile duct epithelium. In various embodiments, the methods comprise administering to the gastrointestinal tract of a subject in need thereof, a genetically engineered microorganism that directs expression of a detection marker specifically in diseased cells. The method further involves detecting the expression of the detection marker to thereby detect the diseased epithelial cells. In various embodiments, the genetically engineered microorganism specifically interacts with diseased epithelial cells through an expressed surface protein that specifically interacts with one or more cell membrane receptor(s) that are specifically present on diseased gastrointestinal epithelial cells (i.e., as compared to non-diseased gastrointestinal epithelial cells) or on diseased bile duct epithelial cells (i.e., as compared to non-diseased bile duct epithelial cells). For example, the cell membrane receptor may not be exposed to the luminal side of epithelial cells of normal gastrointestinal tissue and/or epithelial tissue lining the bile duct, pancreatic duct, or common bile duct, etc., but is exposed to the luminal side of diseased epithelial cells of gastrointestinal tissue and/or epithelial tissue lining the bile duct, pancreatic duct, or common bile duct, etc. in the subject suffering from a disease. The surface protein thereby promotes binding and invasion of the microorganism in the diseased epithelial cells. In various embodiments, the microorganism comprises one or more gene(s) encoding at least one detection marker operably linked to a promoter. Thereby, in various embodiments, the microorganism delivers a nucleic acid (e.g. a DNA or an mRNA molecule) or protein to diseased epithelial cells. In various embodiments, the diseased epithelial cells express the at least one detection marker, and thereby allowing their detection.

In various embodiments, the method disclosed herein detects diseased gastrointestinal (GI) tissue selected from a precancerous lesion, cancer, or a lesion caused by ulcerative colitis, Crohn's disease, Barrett's esophagus, irritable bowel syndrome and/or irritable bowel disease. In any of the embodiments disclosed herein, the detection of the abnormal cells is performed using endoscopy, colonoscopy, MRI, CT scan, PET scan or a combination thereof to detect the detectable marker, and thereby detect the diseased epithelial cells. In various embodiments, the genetically engineered microorganism is administered via oral or rectal route. In various embodiments, optionally a colon cleansing agent may be administered prior to and/or after the administration of the microorganism.

An aspect of the present invention relates to a genetically engineered microorganism. The microorganism comprises a gene encoding a surface protein that specifically interacts with diseased epithelial cells via one or more cell membrane receptor(s) that are exposed to the luminal side of diseased epithelial cells of gastrointestinal tissue and/or epithelial tissue lining the bile duct, pancreatic duct, or common bile duct, etc. The one or more cell membrane receptor(s) are not expressed on the luminal side of epithelial cells of normal gastrointestinal tissue and/or epithelial tissue lining the bile duct, pancreatic duct, or common bile duct, etc., thus conferring the specificity for diseased or abnormal cells of gastrointestinal tissue and/or epithelial tissue lining the bile duct, pancreatic duct, or common bile duct, etc. on the microorganism. The surface protein specifically promotes the invasion of epithelial cells of diseased gastrointestinal tissue and/or epithelial tissue lining the bile duct, pancreatic duct, or common bile duct, etc.

In various embodiments, the microorganism is non-pathogenic. In various embodiments, the microorganism harbors at least one auxotrophic mutation, which optionally includes a deletion, inactivation, or reduced expression or activity of a gene involved in synthesis of a metabolite required for cell wall synthesis. In various embodiments, the at least one auxotrophic mutation facilitates lysis of the microorganism inside the diseased mammalian cell upon invasion. In some embodiments, the auxotrophic mutation is a deletion or inactivation of a gene involved in the synthesis of a metabolite that supports cell wall synthesis. In some embodiments, the gene involved in the synthesis of the metabolite that supports cell wall synthesis is dapA and/or the metabolite that supports cell wall synthesis is diamino pimelic acid. In various embodiments, the microorganism further comprises a gene encoding a lysin, which induces lysis of a phagosome.

In various embodiments, the microorganism comprises one or more gene(s) encoding at least one detection marker operably linked to a promoter. In some embodiments, the promoter is a mammalian promoter that is optionally active or specific for epithelial expression or GI tract epithelial cell-specific expression. In these embodiments, the microorganism delivers a DNA molecule (e.g. a plasmid) to diseased epithelial cells. In these embodiments, the DNA molecule optionally comprises at least one binding site for a DNA binding protein. In some embodiments, the DNA binding protein comprises one or more nuclear localization signal(s) (NLS), thus allowing nuclear translocation of the DNA molecule (e.g. a plasmid) in the diseased epithelial cells. In these embodiments, the diseased epithelial cells express the at least one detection marker from the DNA molecule (e.g. a plasmid) delivered by the microorganism, thereby allowing their detection.

In alternative embodiments, the promoter is a microbial promoter, and the microorganism delivers mRNA to the mammalian cell. In some embodiments, the one or more gene(s) encoding at least one detection marker optionally further comprises an internal ribosome entry site. In these embodiments, the microorganism delivers an mRNA molecule to diseased epithelial cells for translation. In these embodiments, the diseased epithelial cells express the at least one detection marker from the mRNA molecule delivered by the microorganism, thereby allowing their detection.

In alternative embodiments, the promoter is a microbial promoter, and the expressed mRNA is translated in the bacterial cell. In these embodiments, the one or more gene(s) encoding at least one detection marker optionally further comprises a protein that becomes fluorescent upon contact with a metabolite found only in the mammalian cytoplasm. In these embodiments, the microorganism produces and delivers the protein molecules to diseased epithelial cells. In these embodiments, the diseased epithelial cells do not produce the protein, but instead become fluorescent when the protein produced by the microorganism encounters the metabolite found only in the mammalian cytoplasm.

In various embodiments, the detection marker is selected from a fluorescent protein, a bioluminescent protein, a contrast agent for magnetic resonance imaging (MRI), a Positron Emission Tomography (PET) reporter, an enzyme reporter, a contrast agent for use in computerized tomography (CT), a Single Photon Emission Computed Tomography (SPECT) reporter, a photoacoustic reporter, an X-ray reporter, an ultrasound reporter, and ion channel reporters (e.g. cAMP activated cation channel), and a combination of any two or more these. In some embodiments, the one or more gene(s) encoding at least one detection marker comprises at least one intron.

In some embodiments, the fluorescent protein is a near-infrared fluorescent protein selected from iRFP670, miRFP670, iRFP682, iRFP702, miRFP703, miRFP709, iRFP713 (iRFP), iRFP720 and iSplit. In some embodiments, the fluorescent protein is iRFP670 (SEQ ID NO: 5).

Additionally or alternatively, in some embodiments, the detection marker is a bioluminescent protein selected from a Ca²⁺ regulated photoprotein, a luciferase, and active variants thereof. In these embodiments, a substrate of the bioluminescent protein may be administered prior to and/or after the administration of the microorganism.

Additionally or alternatively, in some embodiments, the detection marker is a contrast agent for use in MRI (e.g. a protein or peptide that causes the accumulation of magnetic responsive atoms) selected from ferritin, transferrin receptor-1 (TfR1), Tyrosinase (TYR), beta-galactosidase, manganese-binding protein MntR, sodium iodide symporter, E. coli dihydrofolate reductase, norepinephrine transporter, and active variants thereof. In these embodiments, a substrate of the contrast agent for use in MRI (e.g. a source of magnetic responsive atoms) may be administered prior to and/or after the administration of the microorganism.

Additionally or alternatively, in some embodiments, the detection marker is a PET reporter (e.g. a protein or peptide that causes the accumulation of a positron emitting radioisotope) selected from thymidine kinase, deoxycytidine kinase, Dopamine 2 Receptor, estrogen receptor a surface protein binding domain, somatostatin receptor subtype 2, carcinoembryonic antigen, a sodium iodide symporter, E. coli dihydrofolate reductase, a single-chain antibody specific to 1,4,7,10-tetraazacyclododecane-1,4,7,10-tetraacetic acid (DOTA), or a variants thereof. In these embodiments, a PET probe (e.g. a positron emitting radioisotope) may be administered prior to and/or after the administration of the microorganism.

Additionally or alternatively, in some embodiments, the detection marker is an enzyme reporter selected from beta-galactosidase, chloramphenicol acetyltransferase, horseradish peroxidase, alkaline phosphatase, acetylcholinesterase, and catalase. In these embodiments, a substrate of the enzyme reporter may be administered prior to and/or after the administration of the microorganism.

Additionally or alternatively, in some embodiments, the at least one detection marker is a Single Photon Emission Computed Tomography (SPECT) reporter (e.g. a protein or peptide that causes the accumulation of a gamma-ray emitting radioisotope) selected from sodium ion symporter, norepinephrine transporter, sodium iodide symporter, dopamine receptor, and dopamine transporter. In these embodiments, a SPECT probe (e.g. a gamma-ray emitting radioisotope) may be administered prior to and/or after the administration of the microorganism.

In various embodiments, the microorganism is selected from Lactobacillus, Bifidobacterium, Saccharomyces, Enterococcus, Streptococcus, Pediococcus, Leuconostoc, Bacillus, and Escherichia coli. In some embodiments, the microorganism is Escherichia coli (E. coli), such as E. coli Nissle 1917 or a derivative thereof.

In various embodiments, the one or more gene(s) encoding at least one detection marker may be inserted on a natural endogenous plasmid from Escherichia coli Nissle 1917 (i.e. pMUT1, pMUT2, and/or a derivative thereof). In some embodiments, the plasmid comprises a selection mechanism. In some embodiments, the selection mechanism may not require an antibiotic for plasmid maintenance. Accordingly, in some embodiments, the selection mechanism is selected from an antibiotic resistance marker, a toxin-antitoxin system, a marker causing complementation of a mutation in an essential gene, a cis acting genetic element and a combination of any two or more thereof.

An aspect of the present invention relates to a method of diagnosis of a disease in a subject, the method comprising: (i) administering to the gastrointestinal tract of the subject the genetically engineered microorganism disclosed herein, and (ii) detecting the expression of the detection marker to thereby detecting the diseased epithelial cells.

An aspect of the present invention relates to a method of diagnosis and/or treatment of a disease in a subject, the method comprising: (i) administering to the gastrointestinal tract of the subject a genetically engineered microorganism of any of the embodiments disclosed herein; and (ii) detecting the expression of the detection marker to thereby detecting diseased epithelial cells, optionally wherein the method further comprises administering a treatment to the subject.

An aspect of the present invention relates to a method of selecting a subject suffering from or suspected to be suffering from a disease for a treatment, the method comprising: (i) administering to the gastrointestinal tract of the subject a genetically engineered microorganism of any of the embodiments disclosed herein; (ii) detecting elevated expression of the detection marker compared to surrounding normal epithelial cells; and (iii) selecting the subject for treatment if expression of the detection marker is observed compared to surrounding normal epithelial cells.

An aspect of the present invention relates to a method for treating a cancer in a patient, comprising: (i) administering to the gastrointestinal tract of the subject a genetically engineered microorganism of any of the embodiments disclosed herein; (ii) detecting the expression of the detection marker to thereby detecting the diseased epithelial cells; and (iii) administering a treatment if the expression of the detection marker is observed. In various aspects and embodiments, the treatment is surgery or administration of a therapeutic agent selected from the group consisting of a chemotherapeutic agent, a cytotoxic agent, an immune checkpoint inhibitor, an immunosuppressive agent, a sulfa drug, a corticosteroid, an antibiotic and a combination of any two or more thereof.

Other aspects of the present invention provide a genetically engineered microorganism of any of the embodiments disclosed herein for use in the method of the above aspect.

Any aspect or embodiment disclosed herein can be combined with any other aspect or embodiment as disclosed herein.

### BRIEF DESCRIPTION OF THE DRAWINGS

FIG. 1 shows a schematic representation of diseased cells of GI tract epithelium. Basolateral and lumen sides are shown. The middle cell is a diseased cell, which exhibits mislocalized receptors displayed on the lumen side. Other diseased cells may have novel membrane receptors that are normally not found in the cells, including those formed by translocations and other genomic rearrangements.

FIG. 2 shows a schematic representation E. coli Nissle 1917 (EcN) strain. This strain is an exemplary strain useful for producing the genetically engineered bacterium. Chromosome and naturally occurring plasmids pMUT1 (GenBank Accession No. MW240712) and/or the plasmid pMUT2 (GenBank Accession No. CP023342) are represented by circles of different sizes.

FIG. 3 shows a schematic representation of an embodiment of the base strain of the genetically engineered E. coli Nissle 1917 (EcN) strain harboring one or more auxotrophic mutation(s) (shown by X). Exemplary auxotrophic mutations include dapAΔ, alrΔ, and dadXΔ. Such mutations prevent the bacterial cell from propagating in the body or the environment, and thereby aid in containment of the genetically engineered bacterium.

FIG. 4A and FIG. 4B shows growth requirements and growth characteristics of a genetically engineered E. coli Nissle 1917 (EcN) strain harboring dapAΔ, alrΔ, dadXΔ auxotrophic mutations. Double deletion of alr and dadX, the genes that encode alanine racemase, results in a D-alanine auxotrophy. Deletion of dapA results in auxotrophy for diaminopimelic acid. The graph in FIG. 4A demonstrates that the strain grows only when both D-alanine and diaminopimelic acid are added to growth media. The graph in FIG. 4B demonstrates that that when D-alanine and diaminopimelic acid are added to the media, the strain grows similarly to the wild type strain.

FIG. 5 shows a schematic representation of an embodiment of the genetically engineered bacterium E. coli Nissle 1917 (EcN) strain having genes encoding a surface protein and listeriolysin O (SEQ ID NO: 2) integrated in the genome. Exemplary surface proteins are invasin (SEQ ID NO: 1) and a nanobody/receptor binding peptide expressed on a bacterial scaffold. Listeriolysin is expressed to allow escape from the endosome.

FIG. 6 shows a schematic representation of an embodiment of the genetically engineered E. coli Nissle 1917 (EcN) derivative from which the plasmid pMUT1 has been cured. This strain also harbors auxotrophic mutations (shown by X), and interation of genes encoding invasin/nanobody and listeriolysin O (SEQ ID NO: 2) integrated in the genome.

FIG. 7A and FIG. 7B shows results of curing the cryptic plasmids from E. coli Nissle 1917 (EcN). FIG. 7A shows an agarose gel showing the sequential curing of pMut1 and then pMut2. Wild type E. coli Nissle 1917 (EcN) was transformed with a curing plasmid and passaged in the presence of 5 mg/ml ampicillin. Plasmid preparations from wild type E. coli Nissle 1917 (EcN) (lane A), E. coli Nissle 1917 (EcN) cured of pMUT1 (lane B), and E. coli Nissle 1917 (EcN) cured of pMUT1 and pMUT2 (lane C) FIG. 7B shows the results of quantitative PCR confirming the curing of pMUT1 and pMUT2 in the final strain. qPCR was carried out with primers specific to rpoA (chromosomal marker), pMUT1 and pMUT2 and show are the inverse of the cycle number where the amplification passed a set threshold (Cq⁻¹). Data labels are as in FIG. 7A

FIG. 8 shows a schematic representation of a non-limiting embodiment of the genetically engineered bacterium of present disclosure. This strain is an E. coli Nissle 1917 (EcN) derivative harboring one or more auxotrophic mutation(s) (shown by X), further having genes encoding surface protein and listeriolysin O (also referred to herein as Hly; SEQ ID NO: 2) integrated in the genome. This strain does not contain the plasmid pMUT1, but contains the plasmid pSRX, a pMUT1-based derivative, which is selected using complementation of an auxotrophic mutations as the selection mechanism (e.g., complementation of alr and dadX by plasmid borne alr gene). Plasmid pSRX also carries a detection marker, which is exemplified herein by GFP.

FIG. 9A to FIG. 9D show, without being bound by theory, a schematic representation of the method for detecting diseased gastrointestinal (GI) tissue. FIG. 9A shows specific binding of the genetically engineered bacterium to diseased epithelial cells (represented by the middle cell), which show a mislocalized receptor that is displayed on the lumen side of GI tract. Such binding leads to the internalization in the diseased epithelial cells (represented by the middle cell) of the genetically engineered bacterium. FIG. 9B shows bacterial lysis due to attenuation mutation, and lysis of phagosome through the action of LLO. FIG. 9C shows nuclear localization of the plasmid harboring the detection marker upon lysis of the genetically engineered bacterium. FIG. 9D shows expression of the detection marker by the diseased epithelial cells (represented by the middle cell) of GI tract.

FIG. 10 shows the expression of the detection marker (GFP) expressed by bacterial cells after invading SW480 colorectal cancer cells in vitro. A bacterial strain containing mNeonGreen (a green fluorescent protein) without (top panels) or with (bottom panels) an invasin gene was coincubated with SW480 (colorectal cancer derived cell line) for one hour, followed by washing away of extracellular bacteria. The SW480 cells were visualized by fluorescence microscopy (left panels), removed from the plate, and then analyzed by flow cytometry (right panels) to identify the portion of the SW480 cells that were successfully invaded by the bacterial strain.

FIG. 11A and FIG. 11B show bacterial cells constitutively expressing mNeonGreen (a green fluorescent protein) after invasion into cancerous mammalian cells. FIG. 11A shows the images produced by phase contrast microscopy (“Trans”), fluorescence microscopy (GFP), and merge of the two images of mammalian cells treated with bacteria without (left panels) or with (right panels) an invasin gene. FIG. 11B shows a graph showing the extent of invasion as a function of multiplicity of infection (MOI) as determined using flow cytometry.

FIG. 12A to FIG. 12C show the invasion induced by an intimin-invasin fusion protein (SEQ ID NO: 4). FIG. 12A shows a schematic representation of the intimin-invasin fusion protein. Adapted from Hatlem et al., Catching a SPY: Using the SpyCatcher-SpyTag and Related Systems for Labeling and Localizing Bacterial Proteins. IJMS 20: 2129 (2019). FIG. 12B shows bacterial cells constitutively expressing mNeonGreen (a green fluorescent protein) and an intimin-invasin scaffolding after invasion into cancerous mammalian cells in comparison with the bacteria expressing mNeonGreen and an intimin scaffold (SEQ ID NO: 3). The images produced by phase contrast microscopy (“Trans”), fluorescence microscopy (GFP), and merge of the two images of mammalian cells treated with bacteria expressing intimin scaffold alone (SEQ ID NO: 3, left panels) or intimin-invasin fusion protein (SEQ ID NO: 4, right panels). FIG. 12C shows a bar graph showing the extent of invasion by bacteria expressing intimin scaffold alone, the intimin-invasin fusion protein and invasin.

FIG. 13A and FIG. 13B show the ability of the bacteria to differentiate between diseased and non-diseased tissue when expressing invasin. FIG. 13A shows a schematic representation of the experiment conducted for detection of tumor cells in mice. Bacteria expressing invasin and mNeonGreen (GFP) were mixed with bacteria lacking invasin and expressing mScarlet (RFP) were added to the colon of a mouse containing an induced tumor at the distal end of the colon. After 3 hours, the mice were sacrificed, and their colons removed, washed, and subjected to fluorescent microscopy. FIG. 13B shows epifluorescence microscopy images from colon tissue from mice treated with the bacterial mixture showing that bacteria expressing GFP (and thus, invasin) were able to selectively invade only diseased tissue. Bacteria expressing RFP (and thus, lacking invasin) were unable to invade any tissue and were washed out.

FIG. 14A to FIG. 14E show the requirements for efficient delivery of DNA payloads by the engineered microorganisms disclosed herein. FIG. 14A shows, without being bound by theory, a schematic representation of the proposed mechanism of delivery of genetic payloads and a schematic showing the expression of iRFP670 (SEQ ID NO: 5) from a mammalian promoter (CMV promoter) contained on a high copy bacterial plasmid. FIG. 14B shows phase contrast microscopy (“Trans”), fluorescence microscopy (iRFP670) images, and a merge of the two images, demonstrating the expression of iRFP670 by mammalian cells. FIG. 14C shows the quantification of the expression of iRFP670 (SEQ ID NO: 5) by mammalian cells treated with the engineered microorganisms expressing listeriolysin O (Hly; SEQ ID NO: 2) only, invasin (SEQ ID NO: 1) only, or invasin (SEQ ID NO: 1) and listeriolysin O (SEQ ID NO: 2). FIG. 14D is a bar graph that demonstrating that lysis of the engineered microorganisms is required for efficient delivery of genetic payloads. The efficiency of DNA delivery by strains that secrete Hly with the strains that produce Hly lacking the periplasmic sequencing signal (SEQ ID NO: 6) and so remain in the cytoplasm was also compared in the presence or absence of diaminopimelic acid (DAP). Strains lacking invasion, Hly and/or dapA auxotrophy were used as negative controls. FIG. 14E demonstrates that addition of DAP to culture media during delivery of genetic payloads dapA auxotrophic strains reduces the efficiency of delivery of genetic payloads.

FIG. 15A to FIG. 15C show the delivery of DNA payloads by nonlimiting alternative embodiments of engineered microorganisms. FIG. 15A shows that engineered microorganism that secrete listeriolysin O (Hly; SEQ ID NO: 2) can deliver DNA payloads. A bar graph comparing the efficiency of the delivery of DNA payloads by strains that produce Hly in cytoplasm (SEQ ID NO: 6) and those that secrete Hly is shown. FIG. 15B demonstrates that whole cells, lystates, or culture supernatants of the engineered microorganism that retain or secrete listeriolysin O (Hly) are hemolytic under conditions optimal for listerilysin O. FIG. 15C shows that engineered microorganism having a chromosomal integration of invasin gene can deliver DNA payloads. Shown is a bar graph comparing the efficiency of the delivery of DNA payloads by strains having a chromosomal integration of invasin gene to those having an episomal invasin gene.

FIG. 16A to FIG. 16E demonstrate the delivery of RNA payloads by nonlimiting alternative embodiments of engineered microorganisms disclosed herein. FIG. 16A shows the genomic organization of a nonlimiting embodiment of engineered microorganisms used for delivery of RNA payloads. FIG. 16B shows the expression of GFP mRNA by the nonlimiting embodiments of engineered microorganisms shown in FIG. 16A, as determined by qRT-PCR. FIG. 16C shows the expression of iRFP670 (SEQ ID NO: 5) by mammalian cells upon contact with a nonlimiting embodiments of engineered microorganisms similar to the one shown in FIG. 16A. FIG. 16D shows a nonlimiting modification of mRNA comprising a 5′ stem-loop to improve mRNA stability. FIG. 16E shows the expression of iRFP670 (SEQ ID NO: 5) by mammalian cells upon contact with a nonlimiting embodiments of engineered microorganisms of FIG. 16D.

FIG. 17A and FIG. 17B show the non-limiting embodiments of engineered microorganisms useful for the delivery of RNA payloads harboring a dual plasmid system (FIG. 17A) and single plasmid system (FIG. 17B).

### DETAILED DESCRIPTION

Current diagnosis of abnormally growing cells in the gastrointestinal tract is based upon routine colonoscopies that are not always successful in detection of cancerous or pre-cancerous lesions. The ability to visualize and remove abnormal cells and diseased tissue varies depending on the skill of the surgeon and prominence of the polyps or tumors. Certain abnormally growing cells are flat or small in number and therefore, not visualized and removed even by skilled surgeons. The present disclosure provides engineered bacterial cells that have been genetically engineered to recognize and invade abnormal cells to be administered, for example, prior to a colonoscopy for the purpose of visualizing the cells for detection in, for example, luminescence, PET-, and MRI based imaging modalities.

Accordingly, in various aspects, the present invention provides compositions and methods that are useful for detecting diseased gastrointestinal (GI) tissue. An aspect of the present invention relates to a method for detecting diseased epithelial tissue comprising (i) administering to the gastrointestinal tract of a subject in need thereof, a genetically engineered microorganism, and (ii) detecting the expression of a detection marker in cells of the gastrointestinal tissue and/or epithelial tissue lining the bile duct, pancreatic duct, or common bile duct, etc. (or other target tissue described herein) to thereby detect the diseased epithelial cells, wherein the diseased epithelial tissue is selected from gastrointestinal tract epithelium and bile duct epithelium. In some embodiments, the genetically engineered microorganism is non-pathogenic, auxotrophic, and comprises an exogenous gene encoding a surface protein that specifically interacts with one or more cell membrane receptor(s). In some embodiments, the cell membrane receptor is not exposed to the luminal side of epithelial cells of normal gastrointestinal tissue and/or epithelial tissue lining the bile duct, pancreatic duct, or common bile duct, etc., but is exposed to the luminal side of diseased epithelial cells of gastrointestinal tissue and/or epithelial tissue lining the bile duct, pancreatic duct, or common bile duct, etc. in the subject suffering from a disease. The surface protein promotes binding and invasion of the microorganism in the diseased epithelial cells. The microorganism also comprises one or more gene(s) encoding at least one detection marker operably linked to a promoter to drive mammalian or bacterial RNA expression. In some embodiments, the promoter may be a mammalian promoter. In some embodiments, the mammalian promoter directs epithelial-specific expression or GI tract epithelial cell-specific expression. In some embodiments, the promoter is a bacterial promoter (or a bacteriophage promoter that functions in the bacteria), and the resulting mRNA is translatable by the bacterial cell or the mammalian cell. In some embodiments, the genetically engineered microorganism may be administered via oral or rectal route. In this aspect, a colon cleansing agent may optionally be administered prior to and/or after the administration of the microorganism. In any of the embodiments disclosed herein, the detection of the abnormal cells may be performed using endoscopy, colonoscopy, MRI, CT scan, PET scan or a combination thereof.

The gastrointestinal wall surrounding the lumen of the gastrointestinal tract is made up of four concentric layers called mucosa, submucosa, muscular layer, and serosa (if the tissue is intraperitoneal)/adventitia (if the tissue is retroperitoneal), arranged from the lumen outwards. The characteristics of mucosa depends on the organ. For example, the stomach mucosal epithelium is simple columnar, and is organized into gastric pits and glands to deal with secretion. The small intestinal mucosa, which is made of glandular epithelium intermixed with secretory cells (e.g. goblet cells and Paneth cells), immune cells (e.g. dendritic cells and M cells of the gut-associated lymphoid tissue (GALT)), arranged into villi, creating a brush border and increasing the area for absorption.

The epithelial cells of gastrointestinal tract form a polarized continuous layer. The epithelial cells are connected by tight and adherens junctions, creating a barrier at the apical surface, which controls the selective diffusion of solutes, ions and proteins between the apical and basal tissue compartments. The apical surface of the cells faces the GI tract lumen, and the basolateral surface sits adjacent to an internal-facing basement membrane. The basement membrane is an extracellular matrix (ECM) that comprises laminins, collagen IV, proteoglycans and nidogen. The epithelial cells interact with the ECM through integrins and the transmembrane proteoglycan dystroglycan, which are integral membrane proteins that bind to ECM components as well as intracellular proteins. β1 integrins, which are widely expressed in epithelial cells, have a central role in establishing their polarity. For example, the binding of integrin to ECM components activates signaling by the integrins, which influences the organization of cytoskeleton, which contributes to cellular polarity.

Disruption of the polarity and barrier function causes disease. For example, following inactivation of tumor suppressor APC, tissue polarity is lost very early during cancer progression. See, e.g. Fatehullah et al., Philos Trans R Soc Lond B Biol Sci. 368(1629): 20130014 (2013). Thus, the mislocalization of integrins at the opposing basal surface domain correlated with loss of epithelial architecture and cancer development. Krishnan et al., Mol Biol Cell 24(6):818-31 (2013). Similarly, pathogens such as enteropathogenic Escherichia coli and Y. pseudotuberculosis disrupt cell polarity and enable the apical migration of basolateral membrane proteins. Muza-Moons et al., Infect Immun. 71(12): 7069-7078 (2003); McCormick et al., Infect Immun. 65(4):1414-21 (1997). Moreover, diseases such as Crohn's disease, untreated celiac disease, irritable bower syndrome, irritable bowel disease feature disruption of the barrier function. Marchiando et al., Annu Rev Pathol 5: 119-144 (2010). Therefore, the detection of mislocalized and/or aberrantly expressed cell surface molecules has great diagnostic value.

A bile duct is a long tube-like structures that carry bile. Small bile ducts are visible in portal triads of liver lobule, which also contain a small hepatic artery branch,? a portal vein branch. The small bile ducts fuse to form larger bile ducts. The larger bile ducts in the hepatic triads coalesce to intrahepatic bile ducts that become the right and left hepatic ducts that fuse at the undersurface of the liver to become the common bile duct. About halfway down the common bile duct, the cystic duct (carrying bile to and from the gallbladder) branches off to the gallbladder. The common bile duct opens into the intestine. The intrahepatic ducts, cystic duct, and the common bile duct are lined by a tall columnar epithelium.

The gallbladder stores bile excreted from the liver. The columnar mucosa is arranged in folds over the lamina propria, allowing expansion. Beneath the lamina propria is a muscularis, and surrounding the gallbladder is a connective tissue layer and serosa. The gallbladder mucosa transports out sodium in the bile, passively followed by chloride and water. Thus, bile excreted by the liver and stored in the gallbladder becomes more concentrated. The muscularis of the gallbladder, contracts under the influence of the hormone cholecystokinin excreted by enteroendocrine cells of the small intestine.

The pancreatic duct, or duct of Wirsung (also, known as the major pancreatic duct), is a duct joining the pancreas to the common bile duct. The pancreatic duct joins the common bile duct just prior to the ampulla of Vater, after which both ducts perforate the medial side of the second portion of the duodenum at the major duodenal papilla. There are many rare anatomical variants as well. Pancreatic ducts are lined by columnar cells with luminal microvilli and glycocalyx and small apical cytoplasmic mucin droplets. In large pancreatic ducts, many epithelial cells also have cilia, which function to aid the downstream movement of exocrine secretions.

Accordingly, in various aspects, the present invention provides compositions and methods that are useful for detecting diseased gastrointestinal (GI) tissue. An aspect of the present invention relates to a method for detecting diseased epithelial tissue comprising (i) administering to the gastrointestinal tract of a subject in need thereof, a genetically engineered microorganism engineered to direct expression of a detectable marker specifically in diseased epithelial cells of the GI tract, and (ii) detecting the expression of a detection marker in cells of the GI tract (or other target tissue) to thereby detecting the diseased epithelial cells, wherein the diseased epithelial tissue is selected from gastrointestinal tract epithelium and bile duct epithelium. In various embodiments, the methods comprise administering to the gastrointestinal tract of a subject in need thereof, a genetically engineered microorganism that directs expression of a detection marker specifically in diseased cells. The method further involves detecting the expression of the detection marker to thereby detect the diseased epithelial cells. In some embodiments, the genetically engineered microorganism is non-pathogenic, auxotrophic, and comprises an exogenous gene encoding a surface protein that specifically interacts with one or more cell membrane receptor(s). The cell membrane receptor is not exposed to the luminal side of epithelial cells of normal gastrointestinal tissue and/or epithelial tissue lining the bile duct, pancreatic duct, or common bile duct, etc., but is exposed to the luminal side of diseased epithelial cells of gastrointestinal tissue and/or epithelial tissue lining the bile duct, pancreatic duct, or common bile duct, etc. in the subject suffering from a disease. Thus, the expression and/or localization of the one or more cell membrane receptor(s) confers the specificity for diseased or abnormal cells of gastrointestinal tissue and/or epithelial tissue lining the bile duct, pancreatic duct, or common bile duct, etc. on the microorganism. The surface protein thus promotes binding and invasion of the microorganism in the diseased epithelial cells. The microorganism also comprises one or more gene(s) encoding at least one detection marker operably linked to a promoter (e.g., a mammalian or bacterial promoter). Thereby, in various embodiments, the microorganism delivers a nucleic acid (e.g. a DNA or an mRNA molecule) for expression of the detection marker in diseased epithelial cells. In various embodiments, the diseased epithelial cells express the at least one detection marker, and thereby allowing their detection. In some embodiments, the promoter may be a mammalian promoter. In some embodiments, the mammalian promoter directs GI tract epithelial cell-specific expression. In some embodiments, the promoter is a bacterial promoter, and the resulting mRNA is translatable in the bacterial or mammalian cell.

The diseases that may be diagnosed using the genetically engineered microorganisms, and/or using the methods disclosed herein include precancerous lesions, GI tract cancers, ulcerative colitis, Crohn's disease, Barrett's esophagus, irritable bowel syndrome and irritable bowel disease. GI tract cancers and precancerous syndromes include squamous cell carcinoma of anus, colorectal cancer (CRC, including colorectal adenocarcinoma, familial adenomatous polyposis, hereditary nonpolyposis colorectal cancer), colorectal polyposis (including Peutz-Jeghers syndrome, juvenile polyposis syndrome, MUTYH-associated polyposis, familial adenomatous polyposis/Gardner's syndrome, and Cronkhite-Canada syndrome), carcinoid, pseudomyxoma peritonei, duodenal adenocarcinoma, distal bile duct carcinomas, pancreatic ductal adenocarcinomas, gastric carcinoma, signet ring cell carcinoma (SRCC), gastric lymphoma (MALT lymphoma), linitis plastic (Brinton's disease), and squamous cell carcinoma of esophagus and adenocarcinoma. The diseased epithelial cells from subjects suffering from one or more of these indications may be diagnosed using the genetically engineered microorganisms of the present invention. The genetically engineered microorganisms specifically bind to diseased epithelial cells by specifically interacting with one or more cell membrane receptor(s) that are exposed to the luminal side of diseased epithelial cells of gastrointestinal tissue and/or epithelial tissue lining the bile duct, pancreatic duct, or common bile duct, etc. The genetically engineered microorganisms do not bind to normal (non-diseased) epithelial cells because the one or more cell membrane receptor(s) are not exposed to the luminal side of the normal epithelial cells of gastrointestinal tissue and/or epithelial tissue lining the bile duct, pancreatic duct, or common bile duct, etc. In some embodiments, the genetically engineered microorganism of delivers a one or more nucleic acid(s) encoding at least one detection marker to the diseased epithelial cells (target cells). In these embodiments, the diseased epithelial cells (target cells) express the at least one detection marker, allowing their detection. For example, the diseased epithelial cells (target cells) can be identified as the cells that accumulate the at least one detection marker inside them or on their surface, while the detection marker is not present in or on the surface of the surrounding healthy cells (normal epithelial cells). Detection of the diseased epithelial cells may be carried out using a suitable technique such as colonoscopy, endoscopy, magnetic resonance imaging, CT scan, PET scan, SPECT scan, etc.

Colorectal cancer (CRC) is a common and often lethal tumor. Colorectal adenoma is the most frequent precancerous lesion. Other potentially premalignant conditions include chronic inflammatory bowel diseases and hereditary syndromes, such as familial adenomatous polyposis, Peutz-Jeghers syndrome and juvenile polyposis. These conditions can involve different sites of the gastrointestinal tract. In all such cases, disease recognition at an early stage is essential to devise suitable preventive cancer strategies.

Colorectal adenoma is an asymptomatic lesion often found incidentally during colonoscopy performed for unrelated symptoms or for CRC screening. About 25% men and 15% women who undergo colonoscopic screening have one or more adenomas. Up to 40% of people over the age of 60 harbor colorectal adenomatous polyps as shown in the colonoscopy examinations, although not all colonic polyps are adenomas and more than 90% of adenomas do not progress to cancer.

Lynch syndrome, also known as hereditary non-polyposis colon cancer (HNPCC), accounts for 2-4% of all CRC cases. Individuals with HNPCC have about 75% lifetime risk of developing CRC, and are predisposed to several types of cancer. Colon cancers and polyps arise in Lynch syndrome patients at a younger age than in the general population with sporadic neoplasias, and the tumors develop at a more proximal location. These cancers are often poorly differentiated and mucinous. Muir-Torre syndrome is a variant of Lynch syndrome that presents additional predisposition to certain skin tumors.

Familial adenomatous polyposis (FAP), having a prevalence of 1 in 10,000 individuals, is the second most common genetic syndrome predisposing to CRC. The lifetime risk of developing CRC for individuals suffering from FAP without prophylactic colectomy approaches 100%. The characteristic features of FAP include the development of hundreds to thousands of colonic adenomas beginning in early adolescence. The average age of CRC diagnosis (if untreated) in FAP patients is 40 years; 7% develop the tumor by the age of 20 and 95% by the age of 50. Attenuated FAP is a less severe form of the disease, with an average lifetime risk of CRC of 70%. In this group, approximately 30 adenomatous polyps develop in the colon, colonic neoplasms tend to be located in the proximal colon, and cancer occurs at an older age. Gardner's syndrome and Turcot's syndrome are rare variants of FAP. In addition to polyps, Gardner's syndrome causes extra-colonic symptoms like epidermoid cysts, osteomas, dental abnormalities and/or desmoid tumors. Turcot's syndrome causes colorectal adenomatous polyps, and predisposition to developing malignant tumors of the central nervous system, such as medulloblastoma.

The genetic conditions MUTYH-associated polyposis, Peutz-Jeghers syndrome, and juvenile polyposis syndrome are other rarer syndromes that cause colon polyps, and predisposition to cancer. Patients with MUTYH-associated polyposis (MAP) develop adenomatous polyposis of the colorectum and have an 80% risk of CRC. Peutz-Jeghers and juvenile polyposis syndromes exhibit an increased risk for colorectal and other malignancies with the lifetime risk of CRC is approximately 40%.

Biliary tract cancers, also called cholangiocarcinomas, refer to those malignancies occurring in the organs of the biliary system, including pancreatic cancer, gallbladder cancer, and cancer of bile ducts. Approximately 7,500 new cases of biliary tract cancer are diagnosed each year. These cancers include about 5,000 gallbladder cancers, and between 2,000 and 3,000 bile duct cancers. The preneoplastic and neoplastic lesions of the bile duct and pancreas share analogies in terms of molecular, histological and pathophysiological features. Intraepithelial neoplasms are reported in biliary tract, as biliary intraepithelial neoplasm (BilIN), and in pancreas, as pancreatic intraepithelial neoplasm (PanIN). Both can evolve to invasive carcinomas, respectively cholangiocarcinoma (CCA) and pancreatic ductal adenocarcinoma (PDAC).

BillNs are usually encountered in the epithelium lining the extrahepatic bile ducts (EHBDs), and large intrahepatic bile ducts (IHBDs), and may also be found in the gallbladder. BilINs are microscopic lesions, with a micropapillary, pseudopapillary or flat growth pattern, involved in the process of multistep cholangiocarcinogenesis. Based on the degree of cellular and architectural atypia, BilINs have been classified into three categories: BilIN-1 (low grade dysplasia) showing the mildest changes compared to non-neoplastic epithelium of the bile ducts; BilIN-2 (intermediate grade dysplasia) with increased nuclear atypia and focal anomalies of cellular polarity as compared to BilIN-1; BilIN-3 (high grade dysplasia or carcinoma in situ), which are usually identified in proximity of cholangiocarcinoma areas.

About 30,000 new cases of pancreatic cancer are diagnosed in the United States each year. Because the early symptoms are vague, and there are no screening tests to detect it, early diagnosis is difficult. The pancreatic intraepithelial neoplasm (PanINs) is defined as microscopic flat or micropapillary noninvasive lesions. These lesions are frequently less than 5 mm in size, and considered the most common malignant precursors of pancreatic ductal adenocarcinoma (PDAC). A lower proportion of cases of PDAC also originate from the intraductal papillary mucinous neoplasms of the pancreas (IPMNs) and mucinous cystic neoplasms (MCNs). PanlNs have also been classified, according to the degree of cellular and architectural atypia, into low grade (previously classified as PanIN-1 and PanIN-2) with mild-moderate cytological atypia and basally located nuclei, and high grade (previously classified PanIN-3) with severe cytological atypia, loss of polarity and mitoses.

Inflammatory bowel disease (IBD) is a group of nonspecific chronic inflammatory diseases of the gut, which includes Crohn's disease (CD), ulcerative colitis (UC) and indeterminate colitis. The pathogenesis of IBD remains unclear, and it is characterized by long-lasting and relapsing intestinal inflammation. The incidence of UC in the United States is estimated to be between 9 and 12 per 100,000 persons with a prevalence of 205 to 240 per 100,000 persons (Tally et al., Am J Gastroenterol. 106 Suppl 1:S2-S25 (2011)). The etiology of UC is unknown. However, abnormal immune responses to contents in the gut, including intestinal microbes, are thought to drive disease in genetically predisposed individuals (Geremia et al., Autoimmun Rev. 13:3-10 (2014)). Colitis-associated colorectal cancer (CACC) is one of the most serious complications of inflammatory bowel disease (IBD), particularly in ulcerative colitis (UC); it accounts for approximately 15% of all-causes mortality among IBD patients. Because of worse prognosis and higher mortality in CACC than in sporadic CRC, early CACC detection is crucial.

Crohn's disease is marked by inflammation of the gastrointestinal (GI) tract. The inflammation can appear anywhere in the GI tract from the mouth to the anus. People with the disease often experience ups and downs in symptoms. They may even experience periods of remission. The length of diagnostic delay can represent an issue for at least a proportion of patients with Crohn's disease [CD]. However, Crohn's is a progressive disease that starts with mild symptoms and gradually gets worse. Early diagnosis is important to help prevent bowel damage such as fistulae, abscesses, or strictures.

Irritable bowel syndrome (IBS) is a disorder which manifests as a set of chronic gastrointestinal (GI) symptoms and changes in bowel habits in the absence of evident structural and biochemical abnormalities. IBS has a global prevalence of 10-15% and is more frequent among individuals aged <50 years old. Altered bowel habits are the most commonly reported clinical feature, with the syndrome predominantly associated with constipation (IBS-C), diarrhoea (IBS-D) or a mixture of both conditions (IBS-M). In addition, patients with IBS often experience abdominal pain, which can be provoked by emotional stress or eating and is usually alleviated by the passing of stool. A diagnosis of IBS is confirmed according to the latest version of the Rome criteria based on the clinical experience and consensus of a committee of multinational experts.

Barrett's esophagus is a condition in which tissue that is similar to the lining of intestine replaces tissue lining the esophagus. People with Barrett's esophagus may develop esophageal adenocarcinoma. The exact cause of Barrett's esophagus is unknown, but gastroesophageal reflux disease (GERD) increases the risk developing Barrett's esophagus.

Diagnosis, and specifically early diagnosis is a key for preventing mortality and morbidity in individuals suffering from precancerous lesions, GI tract cancers, ulcerative colitis, Crohn's disease, Barrett's esophagus, irritable bowel syndrome and/or irritable bowel disease.

In various aspects, the present invention provides a genetically engineered microorganism useful in the detection of the mislocalized and/or aberrantly expressed cell surface molecules in the gastrointestinal tract, and thereby diagnose, prognose, or evaluate a disease condition. The genetically engineered microorganism disclosed herein comprises a gene encoding a surface protein, wherein the surface protein specifically interacts with one or more cell membrane receptor(s), wherein the one or more cell membrane receptor(s) are not exposed to the luminal side of epithelial cells of normal gastrointestinal tissue and/or epithelial tissue lining the bile duct, pancreatic duct, or common bile duct, etc.; and wherein the one or more cell membrane receptor(s) are exposed to the luminal side of epithelial cells of diseased gastrointestinal tissue and/or epithelial tissue lining the bile duct, pancreatic duct, or common bile duct, etc. In this aspect, in some embodiments, the surface protein promotes binding and invasion of epithelial cells of diseased gastrointestinal tissue and/or epithelial tissue lining the bile duct, pancreatic duct, or common bile duct, etc. by the genetically engineered microorganism disclosed herein. The microorganism also comprises one or more gene(s) encoding at least one detection marker, which is operably linked to a promoter. In some embodiments, the microorganism may be non-pathogenic and/or harbors at least one auxotrophic mutation. In some embodiments, the at least one auxotrophic mutation includes a deletion, inactivation, or decreased expression or activity of a gene involved in the synthesis of a metabolite (e.g., a non-genetically encoded amino acid) required for cell wall synthesis. In exemplary embodiments, the gene is required for synthesis of D-alanine or diaminopimelic acid. Such auxotrophic mutations provide a means for selection for the engineered microorganism, and also facilitate lysis of the microorganism once inside the mammalian cell.

In some embodiments, the genetically engineered microorganism of the present disclosure delivers a nucleic acid to diseased epithelial cells (target cells). The one or more gene(s) encoding at least one detection marker may include one or more sequence element(s) operably linked to the detection marker genes that control the expression of at least one detection marker. The sequence element may control and regulate the transcription, transcript stability, translation, protein stability, cellular localization, and/or secretion of the detection marker. In some embodiments, the sequence element may prevent expression of the detection marker by the genetically engineered microorganism. In alternative embodiments, the sequence element may allow expression (transcription and/or translation) of the detection marker by the genetically engineered microorganism.

In some embodiments, the genetically engineered microorganism of the present disclosure delivers a DNA molecule (e.g. a plasmid DNA, which is also referred to herein as a payload plasmid) to diseased epithelial cells (target cells). In some embodiments, the payload plasmid is present in multiple copies (ranging from about 1 to about 300 copies, from about 20 to about 50 copies, from about 2 to about 10 copies, or from about 5 to about 10 copies) per cell, or is a single copy plasmid. Copy number depends on the particular genetic characteristics of the plasmid. In some embodiments, the payload plasmid harbors one or more gene(s) encoding at least one detection marker. In some embodiments, the one or more gene(s) encoding at least one detection marker is operably linked to a mammalian promoter. In some embodiments, the one or more gene(s) encoding at least one detection marker comprises a microbial repressor binding site(s) to inhibit bacterial transcription. In some embodiments, the one or more gene(s) encoding at least one detection marker comprises intron(s), where removal of the introns is necessary for functional expression of the detection marker. In some embodiments, the one or more gene(s) encoding at least one detection marker comprises microbial transcription terminator(s).

In some embodiments, the bacteria express a T7 RNA polymerase (T7RNAP) encoded by a T7RNAP gene, and harbor a gene encoding a detection marker disclosed herein under the control of a T7 promoter. In some embodiments, the T7RNAP is integrated on the bacterial chromosome. In some embodiments, the T7RNAP is present on a plasmid. In some embodiments, the T7RNAP is controlled by an inducible promoter (e.g. araBAD or lacUV5 promoters). In these embodiments, the bacteria express mRNA encoding the detection marker and/or the detection marker. In some embodiments, these bacteria deliver mRNA encoding the detection marker to diseased epithelial cells. In these embodiments, the mRNA encoding the detection marker that is delivered to diseased epithelial cells comprises an internal ribosome entry site (IRES). In some embodiments, these bacteria deliver the detection marker protein to diseased epithelial cells. In these embodiments, the detection marker that is delivered to diseased epithelial cells becomes fluorescent upon contact with a cellular metabolite.

Without being bound by theory, it is believed that certain optional sequence elements present in the gene encoding the detection marker (e.g. mammalian promoters, microbial repressor binding sites (e.g. operators), internal ribosome entry sites, and introns) allow production of the detection marker in mammalian cells, while preventing the expression of the detection marker in the genetically engineered microorganism. Therefore, in some embodiments, the genetically engineered microorganism provides a true readout of the presence of diseased epithelial cells (target cells), without background expression in the genetically engineered microorganism. Accordingly, in some embodiments, the one or more gene(s) encoding at least one detection marker may be operably linked to a mammalian promoter. In some embodiments, the mammalian promoter directs GI tract epithelial cell-specific expression. Illustrative examples of suitable mammalian promoters that direct GI tract epithelial cell-specific expression are MUC2 gene promoter, T3^{b} gene promoter, intestinal fatty acid binding protein gene promoter, lysozyme gene promoter and villin gene promoter. In some embodiments, the mammalian promoter directs an inducible GI tract epithelial cell-specific expression. Illustrative example of suitable inducible mammalian promoter may be a cytochrome P450 promoter element that is transcriptionally up-regulated in response to a lipophilic xenobiotic such as β-napthoflavone. In some embodiments, the inducible mammalian promoter may be regulated by tetracycline, cumate, or an estrogen. In some embodiments, the inducible mammalian promoter may be a Tet-On or Tet-Off promoter. Accordingly, in some embodiments, the one or more gene(s) encoding at least one detection marker may be inducible and/or repressible, and optionally controlled by delivering the inducer or repressor to the patient

The microbial repressor binding sites, which are optionally present in the one or more gene(s) encoding at least one detection marker repress the expression of the one or more gene(s) encoding at least one detection marker in bacteria, while exerting no such repressive effect in mammalian cells. In some embodiments, the repressor sequence may be selected from one or more lac operator(s), one or more ara operator(s), one or more trp operator(s), one or more SOS operator(s), one or more integration host factor (IHF) binding sites, one or more histone-like protein HU binding sites, and a combination of two or more thereof.

The microbial transcription termination site(s) cause premature termination of the transcription of the one or more gene(s) encoding at least one detection marker in the genetically engineered microorganism, without causing premature termination of the transcription of the one or more gene(s) encoding at least one detection marker in mammalian cells. In some embodiments, the one or more gene(s) encoding at least one detection marker comprises a rho-independent microbial transcription termination site. In some embodiments, the one or more gene(s) encoding at least one detection marker comprises a 5′ untranslated region, the 5′ untranslated region comprises a rho-independent microbial transcription termination site. In some embodiments, the rho-independent microbial transcription termination site comprises a short hairpin followed by a run of 4-8 Ts (e.g. TTTTTT and TTTTT). Illustrative rho-independent microbial transcription termination sites are T7 terminator, rrnB terminator, and T0 terminator.

In alternative embodiments, the genetically engineered microorganism of the present disclosure may deliver an mRNA molecule encoding at least one detection marker to the diseased epithelial cells (target cells). Accordingly, in these embodiments, the one or more gene(s) encoding at least one detection marker may be operably linked to a microbial promoter (e.g. proD promoter). In some embodiments, the microorganism delivers an mRNA encoding the at least one detection marker to the cytoplasm of diseased epithelial cells. In some embodiments, the one or more gene(s) encoding at least one detection marker comprises an internal ribosome entry site(s) (IRES). In these embodiments, the internal ribosome entry site promotes translation of the mRNA molecule delivered by the microorganism. In some embodiments, the mRNA sequence that is delivered comprises an element that imparts stability on the mRNA molecule. Non-limiting examples of the elements that impart stability on the mRNA molecule include 5′ hairpin structures and 3′poly A tails.

Accordingly, in these embodiments, the one or more gene(s) encoding at least one detection marker may be operably linked to a microbial promoter. Illustrative examples of suitable microbial promoter include a natural promoter of any chromosomal gene, plasmid gene, or bacteriophage gene that functions in a microorganism (e.g. E. coli). In some embodiments, the microbial promoter may be a synthetic promoter derived from a promoter consensus sequence. In some embodiments, the microbial promoter may be an inducible promoter. Illustrative examples of suitable inducible microbial promoters are the araBAD and lac promoters. Accordingly, in some embodiments, the one or more gene(s) encoding at least one detection marker may be inducible and/or repressible, and optionally controlled by delivering the inducer or repressor to the patient.

An internal ribosome entry site (IRES) is an RNA element that allows for translation initiation in a cap-independent manner. In some embodiments, the internal ribosome entry site (IRES) may be selected from an IRES from encephalomyocarditis virus (EMCV), an IRES from hepatitis C virus (HCV), and an IRES from cricket paralysis virus (CrPV). In some embodiments, the internal ribosome entry site(s) present in the one or more gene(s) encoding at least one detection marker allows for the production of the at least one detection marker in mammalian cells using an mRNA produced in the genetically engineered microorganism.

An intron(s), which is optionally present in the one or more gene(s) encoding at least one detection marker prevents the expression of the at least one detection marker in bacteria, while allowing expression of the one or more gene(s) encoding at least one detection marker in mammalian cells, irrespective of whether the mRNA encoding the at least one detection marker may be transcribed in the genetically engineered microorganism or a mammalian cell. In some embodiments, the intron may be a spliceosomal intron. In some embodiments, the intron creates a frameshift or premature stop codon in an unspliced mRNA encoding the at least one detection marker. Therefore, in some embodiments, the genetically engineered microorganism provides a true readout of the presence of diseased epithelial cells (target cells), without background expression of the at least one detection marker protein in the genetically engineered microorganism.

In any of the embodiments disclosed herein, the one or more gene(s) encoding at least one detection marker optionally further comprises a sequence element selected from Kozak sequences, 2A peptide sequences, mammalian transcription termination sequences, polyadenylation sequences (pA), leader sequences for protein secretion and a combination of any two or more thereof.

The Kozak sequence is a nucleic acid motif that functions as the protein translation initiation site in most eukaryotic mRNA transcripts. The Kozak sequence present in the one or more gene(s) encoding at least one detection marker improves correct translation initiation. In some embodiments, the Kozak sequence has the following nucleotide sequence: 5′-(GCC)GCCRCCAUGG-3′.

The 2A peptides, where present, function by preventing the synthesis of a peptide bond between the glycine and proline residues found at the end of the 2A peptides, and that the 2A peptides allow production of equimolar levels of multiple proteins from the same mRNA. The 2A peptides become attached to C-terminus upstream protein, while the downstream protein starts with a proline. In some embodiments, the 2A peptide is selected from E2A ((GSG)QCTNYALLKLAGDVESNPGP), F2A ((GSG)VKQTLNFDLLKLAGDVESNP GP), P2A ((GSG)ATNFSLLKQAGDVEENPGP), and T2A ((GSG)EGRGSLLTCGDVEE NPGP). In some embodiments, the GSG sequence (which is included in the parentheses) may be optionally present.

The polyadenylation sequences (pA) cause addition of a polyA tail to mRNA, which is important for the nuclear export, translation, and stability of mRNA. The mammalian transcription termination sequences terminate transcription and promote the addition of polyA tail. In some embodiments, the one or more gene(s) encoding at least one detection marker comprises a sequence element that is both a mammalian transcription termination sequence and a polyadenylation sequence. In some embodiments, the sequence element that may be both a mammalian transcription termination sequence and a polyadenylation sequence is selected from a SV40 terminator, hGH terminator, BGH terminator, and rbGlob terminator.

In some embodiments, the one or more gene(s) encoding at least one detection marker further comprises leader sequences for protein secretion. In some embodiments, the one or more gene(s) encoding at least one detection marker further comprises the necessary upstream sequences for display of the detection marker on mammalian cell surface.

In some embodiments, the one or more gene(s) encoding at least one detection marker comprises codon usage optimized for mammalian expression.

In some embodiments, the genetically engineered microorganism delivers one or more nucleic acid(s) encoding at least one detection marker to the diseased epithelial cells (target cells). In these embodiments, the diseased epithelial cells (target cells) express the at least one detection marker, allowing their detection. For example, diseased epithelial cells (target cells) can be identified as the cells that accumulate the at least one detection marker inside them or on their surface, while the detection marker is not present in or on the surface of the surrounding healthy cells.

In some embodiments, the detection marker is selected from a fluorescent protein, a bioluminescent protein, a contrast agent for magnetic resonance imaging (MRI), a Positron Emission Tomography (PET) reporter, an enzyme reporter, a contrast agent for use in computerized tomography (CT), a Single Photon Emission Computed Tomography (SPECT) reporter, a photoacoustic reporter, an X-ray reporter, an ultrasound reporter (e.g. a bacterial gas vesicle), and ion channel reporters (e.g. a cAMP activated cation channel), and a combination of any two or more these.

In some embodiments, the at least one detection marker is a fluorescent protein. Accordingly, in some embodiments, the genetically engineered microorganism of delivers one or more nucleic acid(s) encoding at least one fluorescent protein to diseased epithelial cells (target cells). In these embodiments, the diseased epithelial cells (target cells) express the at least one fluorescent protein, allowing their detection. In some embodiments, the detection of diseased epithelial cells is performed using an endoscopic procedure, or colonoscopic procedure. Illustrative endoscopic procedures useful in the detection of the diseased epithelial cells (target cells) are white light endoscopic procedure or Laser-Induced Fluorescence Endoscopy (LIFE).

In these embodiments, the florescent protein is expressed by the diseased epithelial cells. In some embodiments, the at least one detection marker is a fluorescent protein selected from GFP, RFP, YFP, Sirius, Sandercyanin, shBFP-N158S/L173I, Azurite, EBFP2, mKalama1, mTagBFP2, TagBFP, shBFP, ECFP, Cerulean, mCerulean3, SCFP3A, CyPet, mTurquoise, mTurquoise2, TagCFP, mTFP1, monomeric Midoriishi-Cyan, Aquamarine, TurboGFP, TagGFP2, mUKG, Superfolder GFP, Emerald, EGFP, Monomeric Azami Green, mWasabi, Clover, mNeonGreen, NowGFP, mClover3, TagYFP, EYFP, Topaz, Venus, SYFP2, Citrine, Ypet, lanRFP-ΔS83, mPapaya1, mCyRFP1, Monomeric Kusabira-Orange, mOrange, mOrange2, mKOκ, mKO2, TagRFP, TagRFP-T, RRvT, mRuby, mRuby2, mTangerine, mApple, mStrawberry, FusionRed, mCherry, mNectarine, mRuby3, mScarlet, mScarlet-I, mKate2, HcRed-Tandem, mPlum, mRaspberry, mNeptune, NirFP, TagRFP657, TagRFP675, mCardinal, mStable, mMaroon1, mGarnet2, iFP1.4, iRFP713 (iRFP), iRFP670, iRFP682, iRFP702, iRFP720, iFP2.0, mIFP, TDsmURFP, miRFP703, miRFP709 and miRFP670.

In some embodiments, the fluorescent protein is a near-infrared fluorescent protein selected from iRFP670, miRFP670, iRFP682, iRFP702, miRFP703, miRFP709, iRFP713 (iRFP), iRFP720 and iSplit. In some embodiments, the fluorescent protein is iRFP670 (SEQ ID NO: 5). iRFP670 requires biliverdin to fluoresce. Since the microorganisms of present disclosure do not make biliverdin, IRFP 670 fluorescence provides evidence that iRFP670 was located in mammalian cells.

Additionally or alternatively, in some embodiments, the at least one detection marker is a bioluminescent protein. Accordingly, in some embodiments, the genetically engineered microorganism of delivers one or more nucleic acid(s) encoding at least one bioluminescent protein to diseased epithelial cells (target cells). In these embodiments, the diseased epithelial cells (target cells) express the at least one bioluminescent protein, allowing their detection.

In some embodiments, the at least one detection marker is a bioluminescent protein selected from a Ca⁺² regulated photoprotein (e.g. aequorin, symplectin, Mitrocoma photoprotein, Clytia photoprotein, and Obelia photoprotein), North American firefly luciferase, Japanese firefly luciferase, Italian firefly luciferase, East European firefly luciferase, Pennsylvania firefly luciferase, Click beetle luciferase, railroad worm luciferase, Renilla luciferase, Gaussia luciferase, Cypridina luciferase, Metridina luciferase, Metrida luciferase, OLuc protein, red firefly luciferase, bacterial luciferase, and active variants thereof.

In some embodiments, the detection of diseased epithelial cells is performed using an endoscopic procedure, or colonoscopic procedure. In some embodiments, a substrate of the at least one bioluminescent protein is administered during or before the endoscopic procedure, or colonoscopic procedure. Illustrative substrates include luciferin, or a pharmaceutically acceptable, analog, derivative or salt thereof. In some embodiments, the administration of the substrate of the at least one bioluminescent protein may be started prior to marker detection by at least about 1 hour, at least about 6 hours, at least about 12 hours, at least about 24 hours, at least about 2 days, at least about 3 days prior to marker detection.

Additionally or alternatively, in some embodiments, the at least one detection marker is a contrast agent for use in magnetic resonance imaging (MRI) (e.g. a protein or peptide that causes the accumulation of magnetic responsive atoms). Magnetic resonance imaging (MRI) aligns atomic nuclei with an external magnetic field, and perturbs them using radio waves. MRI sensors detect the energy released and the relaxation rate of the nuclei as they realign with the magnetic field. Thus, an illustrative MRI assays the relaxation rate of water protons or other elements in vivo. MRI contrast agents improve the visibility of internal body structures (e.g. diseased epithelial cells) in MRI. Without being bound by theory, it is believed that the MRI contrast agents alter the relaxation times of nuclei, leading to the change in MRI signal intensity. For example, paramagnetic metal ion positively alter the relaxation rate of nearby water proton spins.

Accordingly, in some embodiments, the genetically engineered microorganism delivers one or more nucleic acid(s) encoding at least one contrast agent for use in MRI to diseased epithelial cells (target cells). In these embodiments, the diseased epithelial cells (target cells) express the at least one contrast agent for use in MRI, allowing their detection. In some embodiments, least one contrast agent for use in MRI causes the accumulation of a magnetic responsive atom such as transition metal ions (e.g. Cu²⁺, Fe²⁺/Fe³⁺, Co²⁺, and Mn²⁺), or lanthanide metal ions (e.g Eu³⁺, Gd³⁺, Ho³⁺, and Dy³⁺). In illustrative embodiment, the contrast agent for use in magnetic resonance imaging causes sequestration or chelation metal ions (e.g. Fe³⁺) or catalyzes a biochemical reaction that leads to change in accumulation of ions (e.g. cleavage of a caged synthetic Gd³⁺ compound), and thereby allow the detection of the target cells. For example, the target cells are identified as cells that accumulate the at least one contrast agent for use in MRI, while the surrounding healthy cells do not express the at least one contrast agent for use in MRI.

In some embodiments, the at least one detection marker is a contrast agent for use in MRI selected from ferritin, transferrin receptor-1 (TfR1), Tyrosinase (TYR), beta-galactosidase, manganese-binding protein MntR, creatine kinase (CK), Magnetospirillum magnetotacticum magA, divalent metal transporter DMT1, protamine-1 (hPRM1), urea transporter (UT-B), and ferritin receptor Timd2 (T-cell immunoglobulin and mucin domain containing protein 2), sodium iodide symporter, E. coli dihydrofolate reductase, norepinephrine transporter, and active variants thereof.

In some embodiments, the detection of diseased epithelial cells is performed using a magnetic resonance imaging (MRI) procedure. In some embodiments, the magnetic resonance imaging (MRI) procedure is noninvasive. In some embodiments, a substrate of the at least one contrast agent for use in magnetic resonance imaging is administered during or before the MRI procedure. In some embodiments, the substrate of the at least one contrast agent for use in magnetic resonance imaging is a source of magnetic responsive atoms, which are accumulated by the at least one contrast agent for use in magnetic resonance imaging in or on the surface of the diseased epithelial cells. For example, a caged synthetic Gd³⁺ compound comprising a galactoside may be administered when the contrast agent for use in MRI is beta-galactosidase. In some embodiments, the administration of the substrate of the at least one contrast agent for use in magnetic resonance imaging may be started prior to marker detection by at least about 1 hour, at least about 6 hours, at least about 12 hours, at least about 24 hours, at least about 2 days, at least about 3 days prior to marker detection.

Additionally or alternatively, in some embodiments, the at least one detection marker is a positron emission tomography (PET) reporter. A PET reporter is a protein or peptide that causes the accumulation of a positron emitting radioisotope in or on the surface of diseased epithelial cells. Accordingly, in some embodiments, the genetically engineered microorganism delivers one or more nucleic acid(s) encoding at least one PET reporter to diseased epithelial cells (target cells). In these embodiments, the diseased epithelial cells (target cells) express the at least one PET reporter, allowing their detection.

The positron emission tomography (PET) imaging uses radioactive substances to visualize and measure metabolic processes in the body. For example, a positron emitting radioisotope labeled imaging probe (a PET probe) may be administered to a subject in need thereof. A PET probe is a positron emitting radioisotope. The PET reporter disclosed herein causes accumulation of the PET probe within or on the surface of diseased epithelial cells. The unstable nucleus of the PET probe combines with neighboring electrons to produce gamma rays in the opposite direction at 180 degrees with respect to each other. These gamma rays are detected by the ring of detector placed within the donut-shaped body of the scanner. The energy and location of these gamma rays are used to reconstruct the precise location of the PET probe inside the body of the subject and the amount of imaging probe accumulated at every site at any given time.

In some embodiments, the at least one detection marker is a PET reporter selected from thymidine kinase, deoxycytidine kinase, Dopamine 2 Receptor, estrogen receptor a surface protein binding domain, somatostatin receptor subtype 2, carcinoembryonic antigen, a sodium iodide symporter, a single-chain antibody specific to 1,4,7,10-tetraazacyclododecane-1,4,7,10-tetraacetic acid (DOTA), E. coli dihydrofolate reductase, or a variants thereof.

In some embodiments, the PET reporter causes the accumulation of one or more PET probes in or on the surface of the diseased epithelial cells (target cells). In illustrative embodiments, the one or more PET reporter(s) cause the accumulation of the one or more PET probe(s) through binding to a receptor, antibody, an enzyme, or a cellular transport mechanism. In some embodiments, the detection of diseased epithelial cells is performed using a PET imaging procedure. In some embodiments, one or more PET probe(s) are administered during or before the PET imaging procedure. Illustrative PET probes include [¹⁸F]FHBG, [¹⁸F]FEAU, [¹²⁴I]FIAU, [¹⁸F or ¹¹C]BCNA, [¹¹C] β-galactosyl triazoles, [¹⁸F]L-FMAU, [¹⁸F]FESP, [¹¹C]Raclopride, [¹¹C]N-methylspiperone, [¹⁸F]FES, ⁶⁸Ga-DOTATOC, [¹⁸F]fluoropropyl-trimethoprim, Na¹²⁴I, and a ²²⁵Ac-DOTA chelate. In some embodiments, the administration of the PET probe may be started prior to marker detection by at least about 1 hour, at least about 6 hours, at least about 12 hours, at least about 24 hours, at least about 2 days, at least about 3 days prior to marker detection.

Additionally or alternatively, in some embodiments, the at least one detection marker is an enzyme reporter. Accordingly, in some embodiments, the genetically engineered microorganism delivers one or more nucleic acid(s) encoding at least one enzyme reporter to diseased epithelial cells (target cells). In these embodiments, the diseased epithelial cells (target cells) express the at least one enzyme reporter, allowing their detection.

In some embodiments, the enzyme reporter catalyzes a reaction, which may be detected on the basis of change in, e.g., color, fluorescence or luminescence. Such reactions may use chromogenic, fluorigenic or luminogenic substrates, which may be provided locally or systemically at the time of detection of the diseased cells. In some embodiments, the enzyme substrate is colorigenic, luminogenic, and/or fluorigenic. In some embodiments, the at least one detection marker is an enzyme reporter such as beta-galactosidase, chloramphenicol acetyltransferase, horseradish peroxidase, alkaline phosphatase, acetylcholinesterase, and catalase.

In some embodiments, the detection of diseased epithelial cells is performed using an endoscopic procedure or colonoscopic procedure. In some illustrative embodiments, the enzyme reporter is beta-galactosidase, and the substrate is selected from resorufin β-D-galactopyranoside, 5-dodecanoylaminofluorescein di-β-D-galactopyranoside, 5-bromo-4-chloro-3-indolyl β-D-galactopyranoside (X-Gal), and GALACTO-LIGHT PLUS. Accordingly, in some embodiments, the enzyme substrate is administered before the endoscopic procedure or colonoscopic procedure. In some illustrative embodiments, the enzyme reporter is horseradish peroxidase, and the substrate is selected from 3,3′,5,5′-Tetramethylbenzidine (TMB), 3,3′-Diaminobenzidine (DAB), 2,2′-azino-bis(3-ethylbenzothiazoline-6-sulfonic acid) (ABTS), and 5-Amino-2,3-dihydrophthalazine-1,4-dione (luminol). In some illustrative embodiments, the enzyme reporter is chloramphenicol acetyltransferase, and the substrate is BODIPY FL-1-deoxychloramphenicol. In some embodiments, the administration of the substrate of the enzyme reporter may be started prior to marker detection by at least about 1 hour, at least about 6 hours, at least about 12 hours, at least about 24 hours, at least about 2 days, at least about 3 days prior to marker detection.

Additionally or alternatively, in some embodiments, the at least one detection marker is a Single Photon Emission Computed Tomography (SPECT) reporter. A SPECT reporter is a protein or peptide that causes the accumulation of a gamma-ray emitting radioisotope in or on the surface of the diseased epithelial cells. The Single Photon Emission Computed Tomography (SPECT) imaging uses gamma-ray-generating radioactive substances to visualize body structures. For example, a gamma-ray emitting radioisotope labeled imaging probe (a SPECT probe) may be administered to a subject in need thereof. The SPECT reporter disclosed herein cause accumulation of the SPECT probe within or on the surface of diseased epithelial cells. The gamma rays emitted by the SPECT probe are detected by a gamma detector to acquire multiple 2-D images (also called projections), from multiple angles. A computer is then used to apply a tomographic reconstruction algorithm to the multiple projections, yielding a 3-D data set. This data set may then be manipulated to show thin slices along any chosen axis of the body. In some embodiments, the SPECT reporter causes the accumulation of one or more SPECT probes in or on the surface of the diseased epithelial cells (target cells).

Accordingly, in some embodiments, the genetically engineered microorganism of delivers one or more nucleic acid(s) encoding at least one Single Photon Emission Computed Tomography (SPECT) reporter to diseased epithelial cells (target cells). In these embodiments, the diseased epithelial cells (target cells) express the at least one Single Photon Emission Computed Tomography (SPECT) reporter, allowing their detection. In some embodiments, the Single Photon Emission Computed Tomography (SPECT) reporter causes the accumulation of one or more gamma ray-emitting radio labeled ligand (SPECT probe) in or on the surface of the diseased epithelial cells (target cells). In illustrative embodiments, the one or more SPECT reporter(s) cause the accumulation of the one or more SPECT probe(s) through binding to a receptor, antibody, an enzyme, or a cellular transport mechanism. In some embodiments, the Single Photon Emission Computed Tomography (SPECT) reporter is selected from sodium ion symporter, norepinephrine transporter, sodium iodide symporter, dopamine receptor, and dopamine transporter.

In some embodiments, the detection of diseased epithelial cells is performed using a SPECT imaging procedure. In some embodiments, one or more SPECT probe(s) are administered during or before the SPECT imaging procedure. Illustrative SPECT probes include Sodium pertechnetate ([⁹⁹mTc]NaTcO₄), Na¹²³I, Na¹²⁵I, Na¹³¹I, [¹²³I]-NKJ64, [¹²⁵I]-NKJ64, [¹³¹I]-(R)-N-methyl-3-(2-iodophenoxy)-3-phenylpropanamine, [¹²³I]-NKJ64, [¹²⁵I]-(R)-N-methyl-3-(2-iodophenoxy)-3-phenylpropanamine, [¹³¹I]-(R)-N-methyl-3-(2-iodophenoxy)-3-phenylpropanamine, [¹²³I]β-CIT (2β-carbomethoxy-3β-(4-iodophenyl)tropane), [¹²⁵I]β-CIT (2β-carbomethoxy-3β-(4-iodophenyl)tropane), [¹³¹I]β-CIT [¹²³I]-2β-carbomethoxy-3β-(4-iodophenyl)tropane, [¹²⁵I]-2β-carbomethoxy-3β-(4-iodophenyl)tropane, [¹³¹I]-2β-carbomethoxy-3β-(4-iodophenyl)tropane, [¹²³I]-2′-iodospiperone, [¹²⁵I]-2′-iodospiperone, [¹³¹]-2′-iodospiperone, [¹²³I]epidepride, [¹²⁵I]epidepride, [¹³¹I]epidepride, [¹²³I]-5-iodo-7-N-[(1-ethyl-2-pyrrolidinyl)methyl]carboxamido-2,3-dihydrobenzofuran, [¹²⁵I]-5-iodo-7-N-[(1-ethyl-2-pyrrolidinyl)methyl]carboxamido-2,3-dihydrobenzofuran, [¹³¹I]-5-iodo-7-N-[(1-ethyl-2-pyrrolidinyl)methyl]carboxamido-2,3-dihydrobenzofuran. In some embodiments, the administration of the SPECT probe may be started prior to marker detection by at least about 1 hour, at least about 6 hours, at least about 12 hours, at least about 24 hours, at least about 2 days, at least about 3 days prior to marker detection.

Additionally or alternatively, in some embodiments, the at least one detection marker is a photoacoustic reporter. Accordingly, in some embodiments, the genetically engineered microorganism of delivers a one or more nucleic acid(s) encoding at least one photoacoustic reporter to diseased epithelial cells (target cells). In some embodiments, the detection of diseased epithelial cells is performed using an endoscopic procedure, or colonoscopic procedure. Illustrative a photoacoustic reporters are any fluorescent proteins disclosed herein.

In some embodiments, the genetically engineered microorganism of the present disclosure delivers a protein to diseased epithelial cells (target cells). In these embodiments, the one or more gene(s) encoding at least one detection marker is operably linked to a microbial promoter. In some embodiments, the one or more gene(s) encoding at least one detection marker comprises microbial transcription terminator(s). In some embodiments, the one or more gene(s) encoding at least one detection marker comprises Shine-Dalgarno sequence(s) (bacterial ribosome binding site). In some embodiments, the microbial promoter is inducible and/or repressible. In some embodiments, the microbial promoter is constitutive. In some embodiments, the one or more gene(s) encoding at least one detection marker is inserted on a plasmid. In some embodiments, the one or more gene(s) encoding at least one detection marker is stably integrated on the chromosome. In some embodiments, the one or more gene(s) encoding at least one detection marker encodes a protein that becomes fluorescent upon contact with a metabolite found only in the mammalian cytoplasm. In some embodiments, the protein that becomes fluorescent upon contact with a metabolite found only in the mammalian cytoplasm is infrared fluorescent protein (iRFP), which utilizes biliverdin as a cofactor to gain functionality. In some embodiments, the protein that becomes fluorescent upon contact with a metabolite found only in the mammalian cytoplasm is Japanese freshwater eel (Anguilla japonica) UnaG protein, which fluoresces only upon binding to bilirubin. In these embodiments, the diseased epithelial cells do not produce the protein, but instead become fluorescent when the protein produced by the genetically engineered microorganism encounters the metabolite found only in the mammalian cytoplasm.

The genetically engineered microorganism disclosed herein comprises one or more gene(s) encoding a surface protein, wherein the surface protein specifically interacts with one or more cell membrane receptor(s), wherein the one or more cell membrane receptor(s) are not exposed to the luminal side of epithelial cells of normal gastrointestinal tissue and/or epithelial tissue lining the bile duct, pancreatic duct, or common bile duct, etc.; and wherein the one or more cell membrane receptor(s) are exposed to the luminal side of epithelial cells of diseased gastrointestinal tissue and/or epithelial tissue lining the bile duct, pancreatic duct, or common bile duct, etc. In this aspect, in some embodiments, the surface protein promotes the binding and invasion specifically of epithelial cells of diseased gastrointestinal tissue and/or epithelial tissue lining the bile duct, pancreatic duct, or common bile duct, etc. by the genetically engineered microorganism.

In some embodiments, the surface protein comprises an invasin, or a fragment thereof. In some embodiments, the surface protein comprises an intimin, or a fragment thereof. In some embodiments, the surface protein comprises an adhesin, or a fragment thereof. In some embodiments, the surface protein comprises a flagellin, or a fragment thereof. In some embodiments, the surface protein is the invasin is selected from Yersinia enterocolitica invasin, Yersinia pseudotuberculosis invasin, Salmonella enterica PagN, Candida albicans Als3; and/or the intimin is selected from Escherichia albertii intimin (e.g. NCBI accession no. WP_113650696.1), Escherichia coli intimin (e.g. NCBI accession no. WP_000627885), and Citrobacter rodentium intimin (e.g. NCBI accession no. WP_012907110.1). Intimins are discussed in greater details, e.g, in Adu-Bobie et al., Detection of Intimins α, β, γ, and δ, Four Intimin Derivatives Expressed by Attaching and Effacing Microbial Pathogens, J. Clinical Microbiol 36(3): 662-668 (1998), entire contents of which hereby incorporated by reference in their entirety.

In some embodiments, the surface protein is invasin and YadA (Yersinia enterocolitica plasmid adhesion factor). Rickettsia invasion factor RickA (actin polymerization protein), Legionella RaIF (guanine exchange factor), one or more Neisseria invasion factors (e.g. NadA (Neisseria adhesion/invasion factor), OpA and OpC (opacity-associated adhesions)), Listeria InlA and/or InlB, one or more of Shigella invasion plasmid antigens (e.g. IpaA, IpaB, IpaC, IpgD, IpaB-IpaC complex, VirA, and IcsA), one or more of Salmonella invasion factor (e.g. SipA, sipC, SpiC, SigD, SopB, SopE, SopE2, and SptP), Staphylococcus FnBPA and/or FnBPB, one or more Streptococcus invasion factor (ACP, Fba, F2, Sfb1, Sfb2, SOF, and PFBP), an intimin and/or Porphyromonas gingivalis FimB (integrin binding protein fibriae). In some embodiments, the surface protein comprises a fusion protein of the aforementioned surface proteins. In an exemplary embodiment, the surface protein comprises a fusion protein of invasin and intimin. In embodiments, In some embodiments, the surface protein comprises an active fragment of one or more of invasin, YadA, RickA, RaIF, NadA, OpA, OpC, InlA, InlB, IpaA, IpaB, IpaC, IpgD, IpaB-IpaC, VirA, IcsA, SipA, SipC, SpiC, SigD, SopB, SopE, SopE2, SptP, FnBPA, FnBPB, ACP, Fba, F2, Sfb1, Sfb2, SOF, PFBP, and FimB. In some embodiments, the fragment is expressed on the surface of the engineered microorganism disclosed herein, e.g., on an adhesion scaffold.

In some embodiments, the surface protein is a type III secretion system or a component thereof. In some embodiments, the surface protein comprises a peptide or protein that specifically binds to the surface of cancerous and pre-cancerous cells, optionally wherein the protein is selected from a leptin, an antibody, or a fragment thereof (e.g. sdAb, also known as Nanobody® and an scFv fragment), In some embodiments, the surface protein comprises one or more of leptins, antibodies, or fragments thereof Illustrative examples of fragments of antibodies are single-domain antibody (sdAb, also known as Nanobody®) or scFv fragments. In some embodiments, the surface protein comprises a peptide or protein that specifically binds to mislocalized proteins in cancerous tissues or precancerous lesions (polyps or adenomas), tears and erosions (Barett's Esophagus), or inflammatory diseases.

By virtue of the identity of the surface protein, the genetically engineered microorganism disclosed herein may mimic the affinity of the native surface protein. In some embodiments, the genetically engineered microorganism disclosed herein may specifically bind to one or more of oral epithelial cells, buccal epithelial cells of the tongue, pharyngeal epithelial cells, mucosal epithelial cells, endothelial cells of the stomach, intestinal epithelial cells, colon epithelial etc.

In some embodiments, the genetically engineered microorganism disclosed herein comprises a second exogenous gene encoding a lysin that lyses the endocytotic vacuole, and thereby contributes to pore-formation, breakage or degradation of the phagosome. In some embodiments, the lysin is a cholesterol-dependent cytolysin. In some embodiments, the lysin is selected from the group consisting of listeriolysin O, ivanolysin O, streptolysin, perfringolysin, botulinolysin, leukocidin and a mutant derivative thereof. In some embodiments, the lysin is listeriolysin O (SEQ ID NO: 2), or a mutant derivative thereof (without limitation, e.g. SEQ ID NO: 6).

The genetically engineered microorganism of the present technology may be derived from any non-pathogenic microorganism, such as the non-pathogenic microorganisms that are normal flora of human GI tract or the microorganisms that are generally recognized as safe for human consumption via foods like yogurts, cheeses, breads and the like. In some embodiments, the genetically engineered microorganism of any one of the embodiments disclosed herein may be derived from a microorganism selected from Lactobacillus, Bifidobacterium, Saccharomyces, Enterococcus, Streptococcus, Lactococcus, Pediococcus, Leuconostoc, Bacillus, and Escherichia coli. Illustrative species that are suitable for genetically engineering microorganism of any one of the embodiments disclosed herein include Bacillus coagulans, Bifidobacterium adolescentis, Bifidobacterium animalis, Bifidobacterium bifidum, Bifidobacterium breve, Bifidobacterium essencis, Bifidobacterium faecium, Bifidobacterium infantis, Bifidobacterium lactis, Bifidobacterium longum, Bifidobacterium longum subsp. infantis, Bifidobacterium pseudolungum, Lactobacillus acidophilus, Lactobacillus boulardii, Lactobacillus breve, Lactobacillus brevis, Lactobacillus bulgaricus, Lactobacillus casei, Lactobacillus delbrueckii ssp. Bulgaricus, Lactobacillus fermentum, Lactobacillus gasseri, Lactobacillus helveticus, Lactobacillus paracasei, Lactobacillus plantarum, Lactobacillus reuteri, Lactobacillus rhamnosus, Lactobacillus rhamnosus GG, Lactobacillus salivarius, Lactococcus lactis, Streptococcus thermophilus, Pediococcus acidilactici, Enterococcus faecium, Leuconostoc, Carnobacterium, Proprionibacterium, Saccharomyces boulardii, and Escherichia coli.

In some embodiments, the genetically engineered microorganism of any one of the embodiments disclosed herein may be derived from a probiotic Escherichia coli strain such as Escherichia coli Nissle 1917, Escherichia coli Symbioflor2 (DSM 17252), Escherichia coli strain A0 34/86, Escherichia coli O83 (Colinfant). In some embodiments, the genetically engineered microorganism of any one of the embodiments disclosed herein is derived from Escherichia coli Nissle 1917.

In some embodiments, the genetically engineered microorganism of any one of the embodiments disclosed herein is an Escherichia coli Nissle 1917 or a derivative thereof. Escherichia coli Nissle 1917 contains two naturally occurring, stable, cryptic plasmids pMUT1 and pMUT2. In some embodiments, the Escherichia coli Nissle 1917 or the derivative thereof harbors a plasmid pMUT1 and/or a plasmid pMUT2, and/or one or more derivative thereof. In some embodiments, the Escherichia coli Nissle 1917 or the derivative thereof is cured of the plasmid pMUT1 (GenBank Accession No. MW240712) and/or the plasmid pMUT2 (GenBank Accession No. CP023342). In some embodiments, the Escherichia coli Nissle 1917 derivative harbors a derivative of plasmid pMUT1 having wild type alr gene as a selection mechanism. In some embodiments, the Escherichia coli Nissle 1917 derivative harbors a derivative of plasmid pMUT1 having wild type alr gene as a selection mechanism, and genes encoding invasin and/or listeriolysin, or a mutant derivative thereof. In some embodiments, the Escherichia coli Nissle 1917 derivative having mull mutations in alr and dadX genes harbors a derivative of plasmid pMUT1 having wild type alr gene under its own promoter as a selection mechanism, and optionally, genes encoding invasin (SEQ ID NO: 1) and/or listeriolysin O (SEQ ID NO: 2), or a mutant derivative thereof (without limitation, e.g., SEQ ID NO: 6). In some embodiments, a Escherichia coli Nissle 1917 derivative harbors a derivative of plasmid pMUT2 having wild type alr gene under the control of its own promoter as a selection mechanism. In some embodiments, the Escherichia coli Nissle 1917 derivative harbors a derivative of plasmid pMUT2 having wild type alr gene under the control of its own promoter as a selection mechanism, and genes encoding invasin (SEQ ID NO: 1) and/or listeriolysin O (SEQ ID NO: 2), or a mutant derivative thereof. In some embodiments, the Escherichia coli Nissle 1917 derivative having mull mutations in alr and dadX genes harbors a derivative of plasmid pMUT2 having wild type alr gene under the control of its own promoter as a selection mechanism, and optionally, genes encoding invasin and/or listeriolysin O, or a mutant derivative thereof.

The complete genome sequence of Escherichia coli Nissle 1917 is known. Reister et al., J Biotechnol. 187:106-7 (2014). In some embodiments, the Escherichia coli Nissle 1917 or the derivative thereof, the gene encoding the surface protein is integrated at a first genomic site of Escherichia coli Nissle 1917. Additionally, or alternatively, in some embodiments, the second gene encoding the lysin is integrated at the same site or a second genomic site of Escherichia coli Nissle 1917. In some embodiments, the gene encoding the surface protein, and the second gene encoding the lysin are integrated at a single genomic site, optionally the single genomic site is an integration site of a bacteriophage. Alternatively, one or both genes are inserted into a plasmid, which is optionally a naturally occurring plasmid. Thus, in some embodiments, the one or more gene(s) encoding at least one detection marker may be inserted on a natural endogenous plasmid from Escherichia coli Nissle 1917 (i.e. pMUT1, pMUT2, and/or a derivative thereof). In some embodiments, the plasmid comprises a selection mechanism (e.g., an auxotrophic marker such as alr as described). In alternative embodiments, the gene encoding the surface protein is inserted on a plasmid. In some embodiments, the gene encoding the lysin is inserted on the plasmid.

Additionally, or alternatively, in some embodiments, the one or more gene(s) encoding at least one detection marker is integrated at a genomic site, which can be the same or different from the genomic sites used for integration of the gene encoding the surface protein and/or the gene encoding the lysin. In some embodiments, the gene encoding the surface protein, the second gene encoding the lysin and the gene(s) encoding at least one detection marker are integrated at a single genomic site genomic site, optionally the single genomic site is an integration site of a bacteriophage. Alternatively, the gene encoding the detection marker is inserted into a plasmid, which can be a single copy of multi-copy plasmid, and/or may be naturally occurring plasmid. In some embodiments, the one or more gene(s) encoding at least one detection marker is inserted on the plasmid. Additionally, or alternatively, in some embodiments, the one or more gene(s) encoding at least one detection marker is inserted on a second plasmid. In some embodiments, the microorganism is Escherichia coli Nissle 1917 or a derivative thereof and the plasmid or the second plasmid is selected from the plasmid pMUT1, the plasmid pMUT2, and/or a derivative thereof.

In some embodiments, the plasmid and/or the second plasmid comprises a selection mechanism. In some embodiments, the selection mechanism may not require an antibiotic for plasmid maintenance. Accordingly, in some embodiments, the selection mechanism is selected from an antibiotic resistance marker, a toxin-antitoxin system, a marker causing complementation of a mutation in an essential gene, a cis acting genetic element and a combination of any two or more thereof.

In some embodiments, the selection mechanism is a resistance marker to an antibiotic that is not used or is rarely in human or animals for therapy. In some embodiments, the selection mechanism used for selection of the plasmid and/or the second plasmid is an antibiotic resistance marker selected from kanamycin resistance gene, tetracycline resistance gene and a combination thereof. Additionally, or alternatively, in some embodiments, the selection mechanism used for selection of the plasmid and/or the second plasmid is a toxin-antitoxin system selected from a hok/sok system of plasmid R1, parDE system of plasmid RK2, ccdAB of F plasmid, flmAB of F plasmid, kis/kid system of plasmid R1, XCV2162-ptaRNA1 of Xanthomonas campestris, ataT-ataR of enterohemorragic E. coli or Klebsiella, toxIN system of Erwinia carotovora, parE-parD system of Caulobacter crescentus, fst-RNAII from Enterococcus faecalis plasmid AD1, ϵ-ζ system of Bacillus subtilis plasmid pSM19035 and a combination of any two or more thereof. Additionally, or alternatively, in some embodiments, the selection mechanism used for selection of the plasmid and/or the second plasmid is an essential gene encoding an enzyme involved in biosynthesis of an essential nutrient or a substrate (e.g., an amino acid) required for cell wall synthesis; and/or an house-keeping function. Exemplary amino acids required for cell wall synthesis include D-alanine and diaminopimelic acid. In some embodiments, the essential gene is selected from dapA, dapD, murA, alr, dadX, murI, dapE, thyA and a combination of any two or more thereof. In some embodiments, the essential genes are a combination of alr and dadX (both of which encode for alanine racemases). In some embodiments, the essential genes are a combination of alr and dadX, and the plasmid is selected using a functional alr gene (air⁺, e.g. a wild type alr gene) as a selection marker. In some embodiments, the plasmid and/or the second plasmid is selected by complementation of the alr and dadX mutations by a functional alr gene present on the plasmid and/or the second plasmid. In some embodiments, the house-keeping function is selected from infA, a gene encoding a subunit of an RNA polymerase, a DNA polymerase, an rRNA, a tRNA, a cell division protein, a chaperon protein, and a combination of any two or more thereof. Additionally, or alternatively, in some embodiments, the selection mechanism used for selection of the plasmid and/or the second plasmid is a cis acting genetic element such as ColE1 cer locus or par from pSC101.

In some embodiments, when the genetically engineered microorganism delivers an mRNA molecule the one or more gene(s) encoding at least one detection marker to the diseased epithelial cells (target cells), the one or more gene(s) encoding at least one detection marker is integrated in genome of the genetically engineered microorganism. In some embodiments, when the genetically engineered microorganism delivers an mRNA molecule the one or more gene(s) encoding at least one detection marker to the diseased epithelial cells (target cells), the one or more gene(s) encoding at least one detection marker is present on a plasmid.

In alternative embodiments, when the genetically engineered microorganism delivers a DNA molecule (e.g. a plasmid) comprising the one or more gene(s) encoding at least one detection marker to the diseased epithelial cells (target cells), the one or more gene(s) encoding at least one detection marker is present on a plasmid. In some embodiments, the plasmid comprising the one or more gene(s) encoding at least one detection marker further comprises at least one binding site for a DNA binding protein. In some embodiments, the at least one binding site for a DNA binding protein forms an array of multiple adjacent binding sites for the DNA binding protein. In some embodiments, the DNA binding protein comprises one or more nuclear localization signal(s) (NLS). In these embodiments, the DNA binding protein binds the DNA molecule (e.g. a plasmid) and promotes the nuclear translocation of the DNA molecule (e.g. a plasmid) via the one or more nuclear localization signal(s) (NLS). In some embodiments, the NLS is SV40 T antigen NLS sequence (KKKRKV). In some embodiments, the DNA binding protein is NFκB. In some embodiments, the microorganism comprises a gene encoding the DNA binding protein comprises one or more nuclear localization signal(s) (NLS). Without being bound by theory, it is believed that the DNA binding protein comprising one or more nuclear localization signal(s) binds the at least one binding site for the DNA binding protein on the plasmid comprising the one or more gene(s) encoding at least one detection marker and promotes nuclear translocation of the plasmid via the action of one or more nuclear localization signal(s). thus, in these embodiments, the diseased epithelial cells express the at least one detection marker from the DNA molecule (e.g. a plasmid) delivered by the microorganism, thereby allowing their detection.

In some embodiments, the gene encoding the DNA binding protein is genomically integrated, or present on the plasmid, the second plasmid or a third plasmid.

In some embodiments, the microorganism harbors at least one nutritional auxotrophic mutation selected from dapA, dapD, dapE, murA, alr, dadX, murI, thyA, aroC, ompC, and ompF. In some embodiments, the microorganism harbors a combination of dapA, alr and dadX auxotrophic mutations. In some embodiments, a plasmid is selected by complementation of the alr and dadX mutations by a functional alr gene present on the plasmid. In some embodiments, the at least one nutritional auxotrophic mutation facilitates lysis of the microorganism inside the diseased mammalian cell upon invasion. In some embodiments, the dapA auxotrophic mutation acilitates lysis of the microorganism inside the diseased mammalian cell upon invasion.

In some embodiments, about 10³ to about 10¹¹ viable genetically engineered microorganisms are administered to a subject, depending on the species of the subject, as well as the disease or condition that is being diagnosed or treated. In some embodiments, about 10⁵ to about 10⁹ viable genetically engineered microorganisms of the present disclosure are administered to a subject.

The genetically engineered microorganisms of the present disclosure may be administered between 1 and about 50 times prior to detection of the expressed marker. The genetically engineered microorganisms may be administered from about 1 to about 21, or from 1 to about 14, or from about 1 to about 7 times prior to the marker detection. The genetically engineered microorganisms may be administered starting between about 1 hour to about 2 months prior to marker detection. The administration of the genetically engineered microorganisms may be started prior to marker detection by at least about 1 hour, at least about 6 hours, at least about 12 hours, at least about 24 hours, at least about 2 days, at least about 3 days, at least about 5 days, at least about 7 days, at least about 10 days, at least about 15 days, at least about 20 days, at least about 30 days, at least about 40 days, at least about 50 days, or at least about 60 days.

The genetically engineered microorganisms of the present disclosure may be administered by any route as long as they are capable of invading their target cells upon administration and capable of delivery of their payload. The payload that the genetically engineered microorganisms of the present disclosure deliver are generally a nucleic acid molecule encoding a detection marker. In some embodiments, the genetically engineered microorganism of the present technology is administered by oral and/or rectal route.

The genetically engineered microorganisms of the present disclosure are generally administered along with a pharmaceutically acceptable carrier and/or diluent. The particular pharmaceutically acceptable carrier and/or diluent employed is not critical to the present invention. Examples of diluents include a phosphate buffered saline, buffer for buffering against gastric acid in the stomach, such as citrate buffer (pH 7.0) containing sucrose, bicarbonate buffer (pH 7.0) alone (Levine et al., J. Clin. Invest. 79:888-902 (1987); and Black et al., J. Infect. Dis. 155:1260-1265 (1987)), or bicarbonate buffer (pH 7.0) containing ascorbic acid, lactose, and optionally aspartame (Levine et al., Lancet 2(8609):467-70 (1988)). Examples of carriers include proteins, e.g., as found in skim milk, sugars, e.g., sucrose, or polyvinylpyrrolidone. Typically these carriers would be used at a concentration of about 0.1-30% (w/v) but preferably at a range of 1-10% (w/v).

The pharmaceutically acceptable carriers or diluents which may be used for delivery may depend on specific routes of administration. Any such carrier or diluent can be used for administration of the genetically engineered microorganisms of the invention, so long as the genetically engineered microorganisms of the present disclosure are still capable of invading a target cell and delivering the payload that they carry to the target cells. In vitro or in vivo tests for invasiveness can be performed to determine appropriate diluents and carriers. The compositions of the invention can be formulated for oral and/or rectal administration. Lyophilized forms are also included, so long as the genetically engineered microorganisms are invasive and capable of delivering their payload upon contact with a target cell or upon administration to the subject. Techniques and formulations generally may be found in Remington's Pharmaceutical Sciences, Meade Publishing Co., Easton, Pa.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

The pharmaceutical compositions provided herein may be administered rectally in the forms of suppositories, pessaries, pastes, powders, creams, ointments, solutions, emulsions, suspensions, gels, foams, sprays, or enemas. These dosage forms can be manufactured using conventional processes as described in Remington: The Science and Practice of Pharmacy, supra.

Rectal suppositories are solid bodies for insertion into rectum, which are solid at ordinary temperatures but melt or soften at body temperature to release the genetically engineered microorganisms of the present disclosure inside the rectum. Pharmaceutically acceptable carriers utilized in rectal suppositories include bases or vehicles, such as stiffening agents, which produce a melting point in the proximity of body temperature, when formulated with the pharmaceutical compositions provided herein; and antioxidants, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, hydrogels, such as polyvinyl alcohol, hydroxyethyl methacrylate, polyacrylic acid; glycerinated gelatin. Combinations of the various vehicles may be used. Rectal suppositories may be prepared by the compressed method or molding. The typical weight of a rectal suppository is about 2 to about 3 g.

In some embodiments, the genetically engineered microorganisms of the present disclosure are administered as a single composition, or they are administered individually at the same or different times and via the same or different route (e.g., oral and rectal) of administration. In some embodiments, the genetically engineered microorganisms of the present disclosure is provided in a mixture or solution suitable for rectal instillation and comprises sodium thiosulfate, bismuth subgallate, vitamin E, and sodium cromolyn. In some embodiments, a therapeutic composition of the invention comprises, in a suppository form, butyrate, and glutathione monoester, glutathione diethylester or other glutathione ester derivatives. The suppository can optionally include sodium thiosulfate and/or vitamin E. The pharmaceutical compositions may also be formulated in rectal compositions such as suppositories or retention enemas, e.g., containing conventional suppository bases such as cocoa butter or other glycerides.

In some embodiments, the genetically engineered microorganisms of the present disclosure are formulated as an enema formulation. The enema formulation comprises a reducing agent (or any other agent having a similar mode of action). In some embodiments, an enema formulation of the invention comprises the genetically engineered microorganisms. The enema formulation can optionally comprise polysorbate-80 (or any other suitable emulsifying agent), and/or any short chain fatty acid (e.g., a five, four, three, or two carbon fatty acid) as a colonic epithelial energy source, such as sodium butyrate (4 carbons), proprionate (3 carbons), acetate (2 carbons), etc., and/or any mast cell stabilizer, such as cromolyn sodium (GASTROCROM) or Nedocromil sodium (ALOCRIL).

In some embodiments, the composition comprises from about 10⁵ to about 10⁹ viable genetically engineered microorganisms of the present disclosure. If the composition comprises cromolyn sodium it can be present in an amount from about 10 mg to about 200 mg, or from about 20 mg to about 100 mg, or from about 30 mg to about 70 mg. If the composition comprises polysorbate-80, it can be provided at a concentration from about 1% (v/v) to about 10% (v/v). If the composition comprises sodium butyrate it can be present in an amount of about 500 to about 1500 mg. In some embodiments, the composition suitable for administration as an enema is formulated to include genetically engineered microorganisms of the present disclosure, cromolyn sodium, and polysorbate-80. In some embodiments, the composition further comprises alpha-lipoic acid and/or L-glutamine and/or N-acetyl cysteine and/or sodium butyrate (1.1 gm).

The compositions may, if desired, be presented in a pack or dispenser device and/or a kit that may contain one or more unit dosage forms containing the active ingredient. The pack may for example comprise metal or plastic foil, such as a blister pack. The pack or dispenser device may be accompanied by instructions for administration.

In various aspects, the present invention provides a method for detecting diseased epithelial tissue, the method comprising (i) administering to the gastrointestinal tract of a subject in need thereof, a genetically engineered microorganism disclosed herein; and (ii) detecting the expression of the detection marker to thereby detecting the diseased epithelial cells, wherein the diseased epithelial tissue is selected from gastrointestinal tract epithelium and bile duct epithelium. As discussed above, the microorganism comprises an exogenous gene encoding a surface protein, wherein the surface protein specifically interacts with one or more cell membrane receptor(s), which are not exposed to the luminal side of epithelial cells of normal gastrointestinal tissue and/or epithelial tissue lining the bile duct, pancreatic duct, or common bile duct, etc. but is exposed to the luminal side of diseased epithelial cells of gastrointestinal tissue and/or epithelial tissue lining the bile duct, pancreatic duct, or common bile duct, etc. in the subject suffering from a disease. In some embodiments, the surface protein promotes binding and invasion of the microorganism in the diseased epithelial cells. The microorganism also comprises one or more gene(s) encoding at least one detection marker operably linked to a promoter.

In some embodiments, the promoter is a mammalian promoter. In some embodiments, the mammalian promoter that is active or specific for epithelial expression or GI tract epithelial cell-specific expression. In some embodiments, the mammalian promoter directs GI tract epithelial cell-specific expression. In these embodiments, the microorganism delivers a DNA molecule (e.g. a plasmid) to diseased epithelial cells. In some embodiments, the genetically engineered microorganism is administered via oral or rectal route. In some embodiments, the method further comprises administration of a colon cleansing agent comprising a laxative. In some embodiments, the colon cleansing agent comprising the laxative is administered prior to the administration of the microorganism.

The diseased gastrointestinal (GI) tissue may be precancerous lesion(s), a GI tract cancer, ulcerative colitis, Crohn's disease, Barrett's esophagus, irritable bowel syndrome and irritable bowel disease. Illustrative precancerous lesion(s) and GI tract cancers include squamous cell carcinoma of anus, low-grade squamous intraepithelial lesions (LSIL) of anus, high-grade squamous intraepithelial lesions (HSIL) of anus, colorectal cancer, colorectal adenocarcinoma, familial adenomatous polyposis, hereditary nonpolyposis colorectal cancer, colorectal polyposis (e.g. Peutz-Jeghers syndrome, juvenile polyposis syndrome, MUTYH-associated polyposis, familial adenomatous polyposis/Gardner's syndrome, and Cronkhite-Canada syndrome), carcinoid, pseudomyxoma peritonei, duodenal adenocarcinoma, premalignant adenoma of small bowel, distal bile duct carcinomas, biliary intraepithelial neoplasm (BilIN), BilIN-1, BilIN-2, BilIN-3 or cholangiocarcinoma, pancreatic ductal adenocarcinoma (PDAC), pancreatic intraepithelial neoplasm (PanIN), PanIN-1, PanIN-2, PanIN-3, gastric carcinoma, signet ring cell carcinoma (SRCC), gastric lymphoma (MALT lymphoma), linitis plastic (Brinton's disease), and squamous cell carcinoma of esophagus and adenocarcinoma.

In some embodiments, the gastrointestinal (GI) tissue may be potentially diseased because the subject suffers from disease such as a precancerous lesion, cancer, ulcerative colitis, Crohn's disease, Barrett's esophagus, irritable bowel syndrome and irritable bowel disease. In some embodiments, the precancerous lesion comprises a polyp such as a sessile polyp, serrated polyp (e.g. hyperplastic polyps, sessile serrated adenomas/polyps, and traditional serrated adenoma), sessile serrated polyp, flat polyp, sub-pedunculated polyp , pedunculated polyp, and a combination thereof. In some embodiments, the polyp is a diminutive polyp. In some embodiments, the precancerous lesion comprises a biliary intraepithelial neoplasm (BilIN) selected from BilIN-1, BilIN-2, BilIN-3, and cholangiocarcinoma. In some embodiments, the precancerous lesion comprises a pancreatic intraepithelial neoplasm (PanIN) selected from PanIN-1, PanIN-2, PanIN -3 and pancreatic ductal adenocarcinoma (PDAC). In some embodiments, the precancerous lesion has a size from about 0.05 mm to about 30 mm. In some embodiments, the precancerous lesion has a size from less than about 0.1 mm, less than about 0.25 mm, less than about 0.5 mm, less than about 1 mm, less than about 2 mm, less than about 5 mm, less than about 8 mm, less than about 10 mm, less than about 15 mm, less than about 20 mm, less than about 25 mm, less than about 30 mm.

In some embodiments, the cancer comprises a polyp, an adenoma, or a frank cancer. In some embodiments, the cancer comprises Lynch syndrome, familial adenomatous polyposis, hereditary non-polyposis colon cancer (HNPCC), or a sporadic cancer. In some embodiments, the cancer comprises a biliary intraepithelial neoplasm (BilIN), BilIN-1, BilIN-2, BilIN-3 or cholangiocarcinoma), pancreatic intraepithelial neoplasm (PanIN), PanIN-1, PanIN-2, PanIN-3 or pancreatic ductal adenocarcinoma (PDAC).

In some embodiments, the at least one detection marker is selected from a fluorescent protein, a bioluminescent protein, a contrast agent for magnetic resonance imaging (MRI), a Positron Emission Tomography (PET) reporter, an enzyme reporter, a contrast agent for use in computerized tomography (CT), a Single Photon Emission Computed Tomography (SPECT) reporter, a photoacoustic reporter, an X-ray reporter, an ultrasound reporter, and ion channel reporters (e.g. cAMP activated cation channel), and a combination of any two or more thereof. In some embodiments, the fluorescent protein, the bioluminescent protein, the contrast agent for magnetic resonance imaging (MRI), the Positron Emission Tomography (PET) reporter, the enzyme reporter, the contrast agent for use in computerized tomography (CT), the Single Photon Emission Computed Tomography (SPECT) reporter, the photoacoustic reporter, the X-ray reporter, the ultrasound reporter, and the ion channel reporters (e.g. cAMP activated cation channel) of any of the embodiments disclosed herein may be used.

In some embodiments, the method further comprises administration of one or more substrate(s) of the at least one bioluminescent protein, one or more substrate(s) of the at least one contrast agent for use in magnetic resonance imaging, one or more PET probe(s), one or more substrate of the enzyme reporter, one or more SPECT probe(s) or a combination of any two or more thereof. In some embodiments, the administration of one or more substrate(s) of the at least one bioluminescent protein, one or more substrate(s) of the at least one contrast agent for use in magnetic resonance imaging, one or more PET probe(s), one or more substrate of the enzyme reporter, one or more SPECT probe(s) or a combination of any two or more thereof may be started prior to marker detection by at least about 1 hour, at least about 6 hours, at least about 12 hours, at least about 24 hours, at least about 2 days, at least about 3 days prior to marker detection. In some embodiments, the one or more substrate(s) of the at least one bioluminescent protein, one or more substrate(s) of the at least one contrast agent for use in magnetic resonance imaging, one or more PET probe(s), one or more substrate of the enzyme reporter, one or more SPECT probe(s) or a combination of any two or more thereof may be administered after the administration of the microorganism.

In various aspects, the present invention provides a method for diagnosis and/or prognosis of a disease or disorder in a subject, the method comprising (i) administering to the gastrointestinal tract of a subject in need thereof, a genetically engineered microorganism disclosed herein; and (ii) detecting the expression of the detection marker to thereby detecting the diseased epithelial cells. As discussed above, the microorganism comprises an exogenous gene encoding a surface protein, wherein the surface protein specifically interacts with one or more cell membrane receptor(s), which are not exposed to the luminal side of epithelial cells of normal gastrointestinal tissue and/or epithelial tissue lining the bile duct, pancreatic duct, or common bile duct, etc. but is exposed to the luminal side of diseased epithelial cells of gastrointestinal tissue and/or epithelial tissue lining the bile duct, pancreatic duct, or common bile duct, etc. in the subject suffering from a disease. In some embodiments, the surface protein promotes binding and invasion of the microorganism in the diseased epithelial cells. The microorganism also comprises one or more gene(s) encoding at least one detection marker operably linked to a promoter. In some embodiments, the promoter is a mammalian promoter. In some embodiments, the mammalian promoter that is active or specific for epithelial expression or GI tract epithelial cell-specific expression. In some embodiments, the mammalian promoter directs GI tract epithelial cell-specific expression. In these embodiments, the microorganism delivers a DNA molecule (e.g. a plasmid) to diseased epithelial cells. In some embodiments, the genetically engineered microorganism is administered via oral or rectal route. In some embodiments, the method further comprises administration of a colon cleansing agent comprising a laxative. In some embodiments, the colon cleansing agent comprising the laxative is administered prior to the administration of the microorganism. In some embodiments, the genetically engineered microorganism is non-pathogenic. In some embodiments, the genetically engineered microorganism is auxotrophic. In some embodiments, the genetically engineered microorganism is non-pathogenic and auxotrophic.

In various aspects, the present invention provides a genetically engineered microorganism for use in a method of diagnosis and/or prognosis of a disease or disorder in a subject, the method comprising (i) administering to the gastrointestinal tract of a subject in need thereof, disclosed herein; and (ii) detecting the expression of the detection marker to thereby detecting the diseased epithelial cells. As discussed above, the microorganism comprises an exogenous gene encoding a surface protein, wherein the surface protein specifically interacts with one or more cell membrane receptor(s), which are not exposed to the luminal side of epithelial cells of normal gastrointestinal tissue and/or epithelial tissue lining the bile duct, pancreatic duct, or common bile duct, etc. but is exposed to the luminal side of diseased epithelial cells of gastrointestinal tissue and/or epithelial tissue lining the bile duct, pancreatic duct, or common bile duct, etc. in the subject suffering from a disease. In some embodiments, the surface protein promotes binding and invasion of the microorganism in the diseased epithelial cells. The microorganism also comprises one or more gene(s) encoding at least one detection marker operably linked to a promoter. In some embodiments, the promoter is a mammalian promoter. In some embodiments, the mammalian promoter directs GI tract epithelial cell-specific expression. In some embodiments, the genetically engineered microorganism is administered via oral or rectal route. In some embodiments, the method further comprises administration of a colon cleansing agent comprising a laxative. In some embodiments, the colon cleansing agent comprising the laxative is administered prior to the administration of the microorganism. In some embodiments, the genetically engineered microorganism is non-pathogenic. In some embodiments, the genetically engineered microorganism is auxotrophic. In some embodiments, the genetically engineered microorganism is non-pathogenic and auxotrophic.

Disclosed herein, in various aspects, are methods of selecting a subject suffering from or suspected to be suffering from a disease for a treatment, the method comprising: (i) administering to the gastrointestinal tract of the subject a genetically engineered microorganism of any one of embodiments disclosed herein; (ii) detecting elevated expression of the detection marker compared to surrounding normal epithelial cells; and (iii) selecting the subject for treatment if expression of the detection marker is observed compared to surrounding normal epithelial cells. In some embodiments, the disease is selected from a precancerous lesion, cancer, ulcerative colitis, Crohn's disease, Barrett's esophagus, irritable bowel syndrome and irritable bowel disease. In some embodiments, the treatment is surgery or administration of a therapeutic agent. In some embodiments, the surgery removes diseased tissue. In some embodiments, the therapeutic agent is selected from a chemotherapeutic agent, a cytotoxic agent, an immune checkpoint inhibitor, an immunosuppressive agent, a sulfa drug, a corticosteroid, an antibiotic and a combination of any two or more thereof.

In some embodiments, the precancerous lesion comprises a polyp selected from sessile polyp, serrated polyp (e.g. hyperplastic polyps, sessile serrated adenomas/polyps, and traditional serrated adenoma), sessile serrated polyp, flat polyp, sub-pedunculated polyp , pedunculated polyp, and a combination thereof. In some embodiments, the polyp is a diminutive polyp. In some embodiments, the polyp is a diminutive polyp. In some embodiments, the precancerous lesion comprises a biliary intraepithelial neoplasm (BilIN) selected from BilIN-1, BilIN-2, BilIN-3, and cholangiocarcinoma. In some embodiments, the precancerous lesion comprises a pancreatic intraepithelial neoplasm (PanIN) selected from PanIN-1, PanIN-2, PanIN-3 and pancreatic ductal adenocarcinoma (PDAC). In some embodiments, the precancerous lesion has a size of from about 0.05 mm to about 30 mm. In some embodiments, the precancerous lesion has a size of less than about 0.1 mm, less than about 0.25 mm, less than about 0.5 mm, less than about 1 mm, less than about 2 mm, less than about 5 mm, less than about 8 mm, less than about 10 mm, less than about 15 mm, less than about 20 mm, less than about 25 mm, or less than about 30 mm.

Additionally, or alternatively, in some embodiments, the cancer comprises a polyp, an adenoma, or a frank cancer. In some embodiments, the cancer comprises Lynch syndrome, familial adenomatous polyposis, hereditary non-polyposis colon cancer (HNPCC), or a sporadic cancer.

Also disclosed herein, in various aspects, are methods of treating a cancer in a patient. These methods comprise: (i) administering to the gastrointestinal tract of the subject a genetically engineered microorganism of any one of claims 57 to 100; (ii) detecting the expression of the detection marker to thereby detecting the diseased epithelial cells; and (iii) administering a treatment if the expression of the detection marker is observed. In some embodiments, the treatment is surgery or administration of a therapeutic agent. In some embodiments, the therapeutic agent is selected from the group consisting of a chemotherapeutic agent, a cytotoxic agent, an immune checkpoint inhibitor, an immunosuppressive agent, a sulfa drug, a corticosteroid, an antibiotic and a combination of any two or more thereof.

### EXAMPLES

### Example 1

### Genetically Engineered Bacterial Strains and the Setup

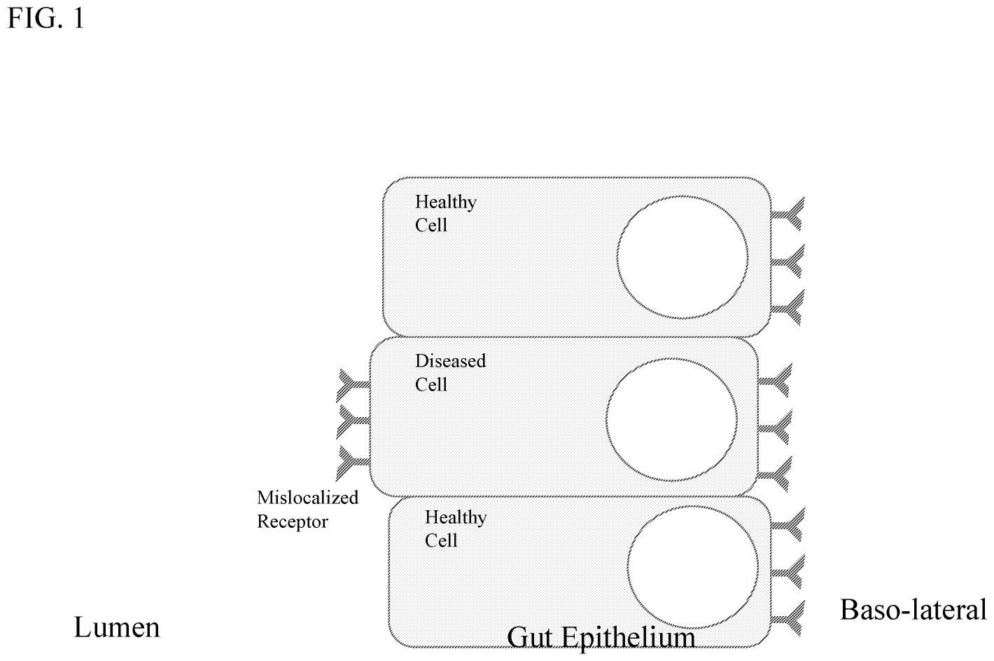

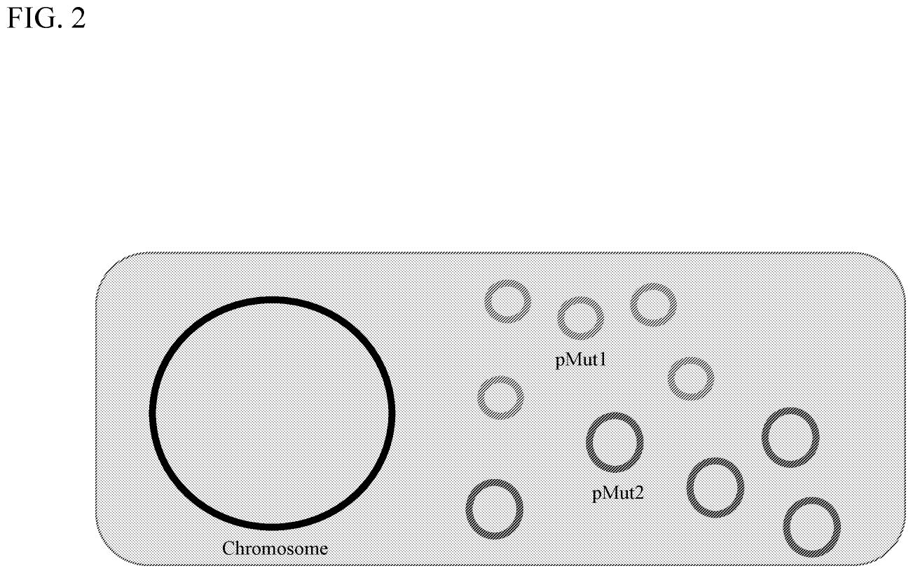

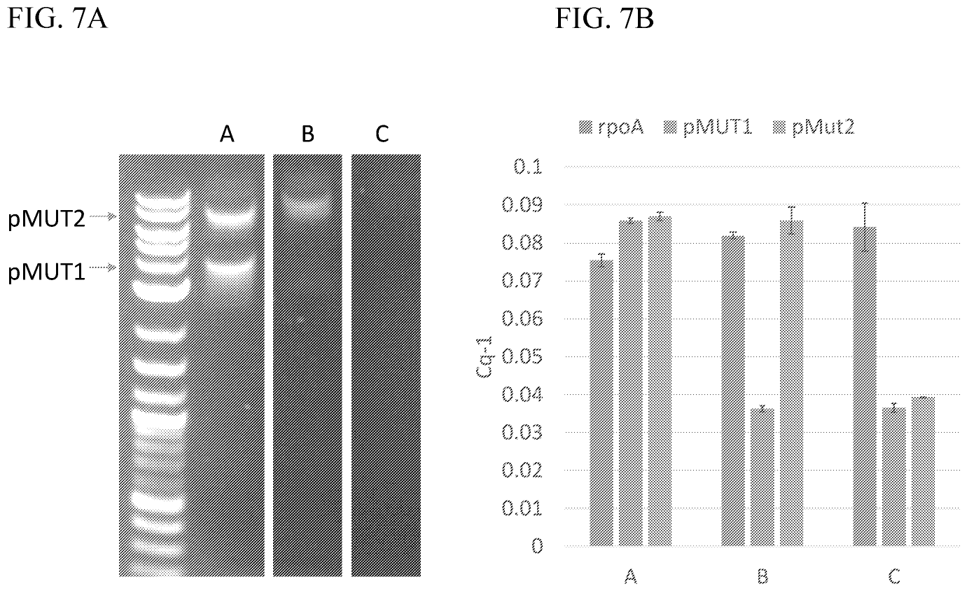

One of the objectives of this study was to construct bacterial strains that can detect diseased cells in gastrointestinal tract epithelium. As shown in FIG. 1, diseased epithelial cells exhibit mislocalized and/or expression of novel mammalian membrane receptors. The novel receptors may be receptors that are not normally found in the cells or receptors formed by translocations and other genomic rearrangement. Disclosed herein are bacterial strains derived from Escherichia coli Nissle 1917. As shown in FIG. 2, the strain contains a single bacterial chromosome and two extra chromosomal plasmids (pMUT1 and pMUT2). See lane A of FIG. 7.

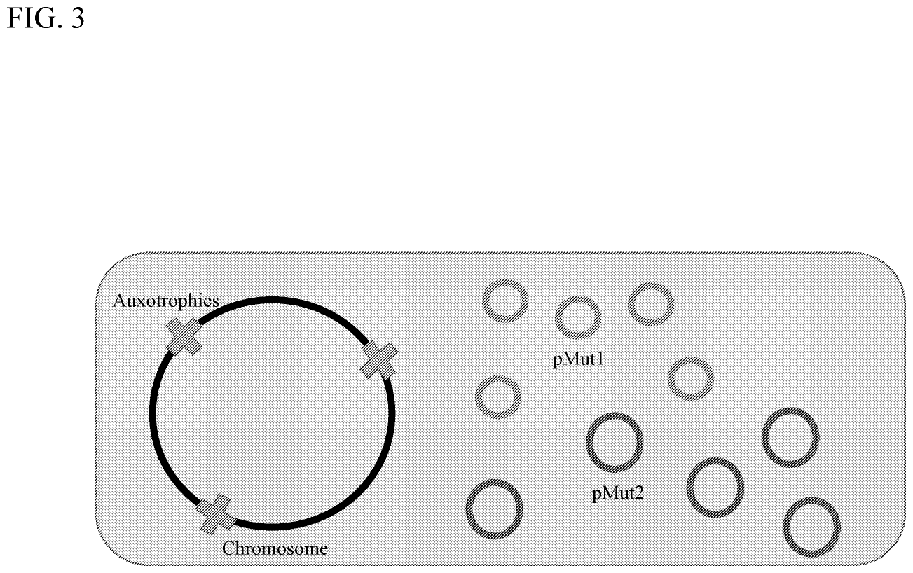

Nutritional auxotrophies were introduced (See FIG. 3) to allow containment of the bacterial strains. The nutritionally auxotrophic strains cannot reproduce in the body or environment. Moreover, the nutritional auxotrophies allow for the antibiotic free selection of the plasmids.

For bacterial containment, dapA gene, which is essential to produce diaminopimelic acid, an essential component of the bacterial cell wall, was knocked out. ΔdapA strains require diaminopimelic acid in the media for growth. For plasmid selection, alr and dadX genes were knocked out. alr and dadX are redundant alanine racemases and render the bacterial strain dependent on being supplied with the amino acid D-Alanine, which is also component of the bacterial cell wall, for growth.

All auxotrophies were generated with the well-established lambda red recombination system and done in such a way as to eliminate the antibiotic marker. Datsenko and Wanner, Proc Natl Acad Sci U S A. 97(12):6640-5 (2000). As a result, the final strain is sensitive to all antibiotics that the E. coli Nissle 1917 strain is sensitive to and is expected to require the addition of diaminopimelic acid and D-alanine for growth.

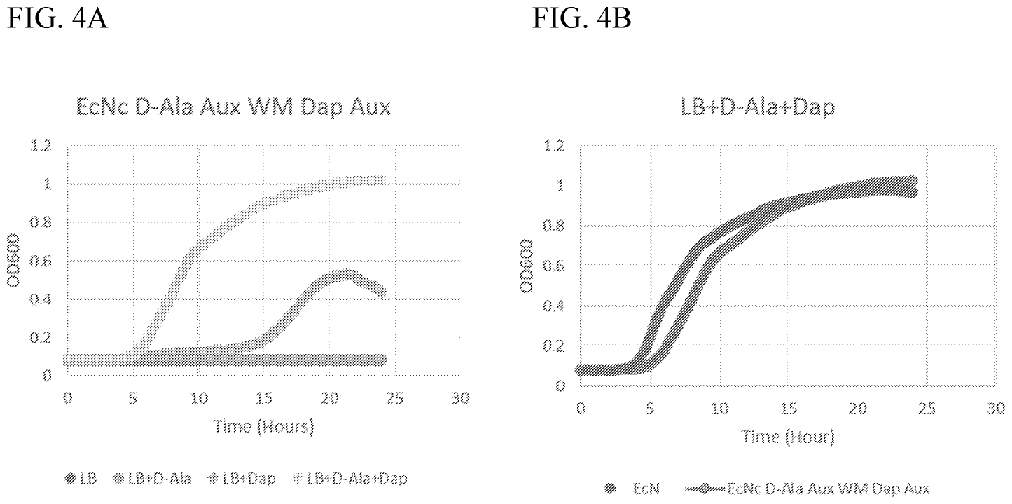

The resultant strain (E. coli Nissle 1917 ΔdapA Δalr ΔdadX) was grown in LB media supplemented with D-alanine and diaminopimelic acid. The cultures were diluted in (1) LB, (2) LB supplemented with D-alanine only, (3) LB supplemented diaminopimelic acid only, and (4) LB supplemented with D-alanine and diaminopimelic acid, incubated at 37° C., and growth was monitored. As shown in FIG. 4A, the strain only grew only when both D-alanine and diaminopimelic acid were added to the media. Further, as shown in FIG. 4B, when D-alanine and diaminopimelic acid were added to the media, the strain exhibited growth properties that were similar to that of the wild type strain.

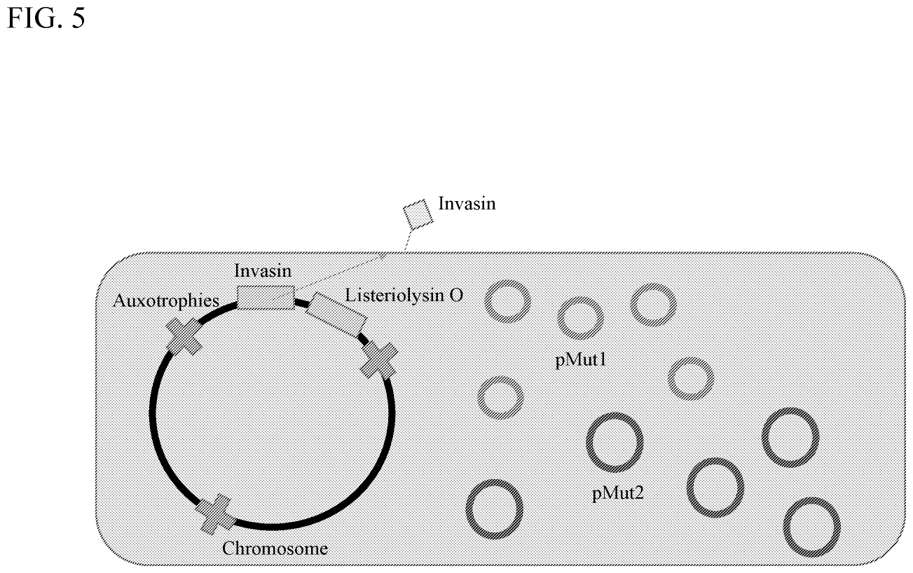

A chassis containing invasin (SEQ ID NO: 1) and listeriolysin O (SEQ ID NO: 2) was created. The invasin and listeriolysin O genes were maintained on a plasmid. Alternatively, a bacterial strain harboring stably integrated invasin (SEQ ID NO: 1) and listeriolysin O (SEQ ID NO: 2) genes can be constructed (FIG. 5).

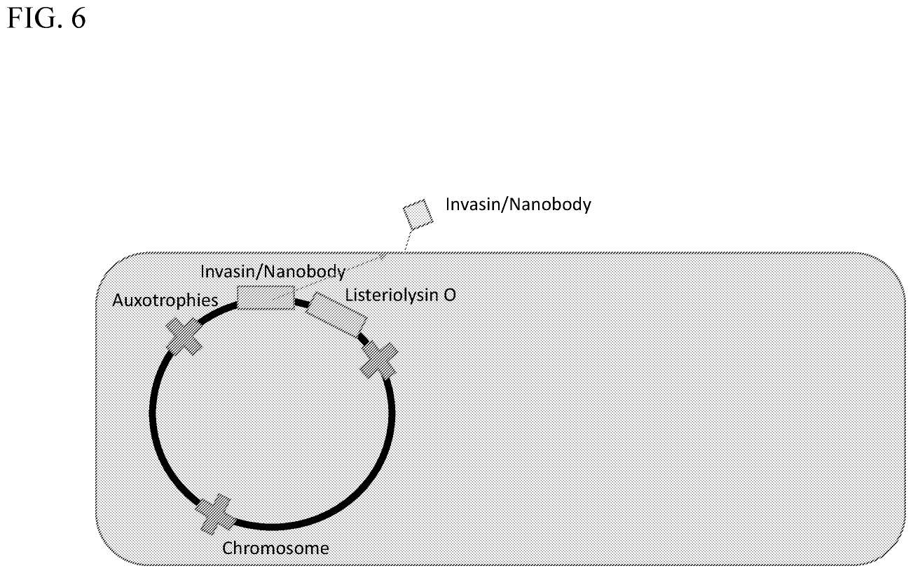

Next, the plasmids pMUT1 and pMUT2 were cured using standard procedures (FIG. 6). FIG. 7A shows an agarose gel showing results of an experiment conducted to cure plasmids pMUT1 and pMUT2 from an E. coli Nissle 1917 (EcN) derivative. Wild type E. coli Nissle 1917 (EcN) was transformed with a curing plasmid and passaged in the presence of 5 mg/ml ampicillin. Plasmid preparations from wild type E. coli Nissle 1917 (EcN) (lane A), E. coli Nissle 1917 (EcN) cured of pMUT1 (lane B), and E. coli Nissle 1917 (EcN) cured of pMUT1 and pMUT2 (lane C) Expected locations of plasmids pMUT1 and pMUT2 are shown. FIG. 7B shows the results of a quantitative PCR experiment to confirm that the plasmids have been cured. Data labels are the same as in FIG. 7A.

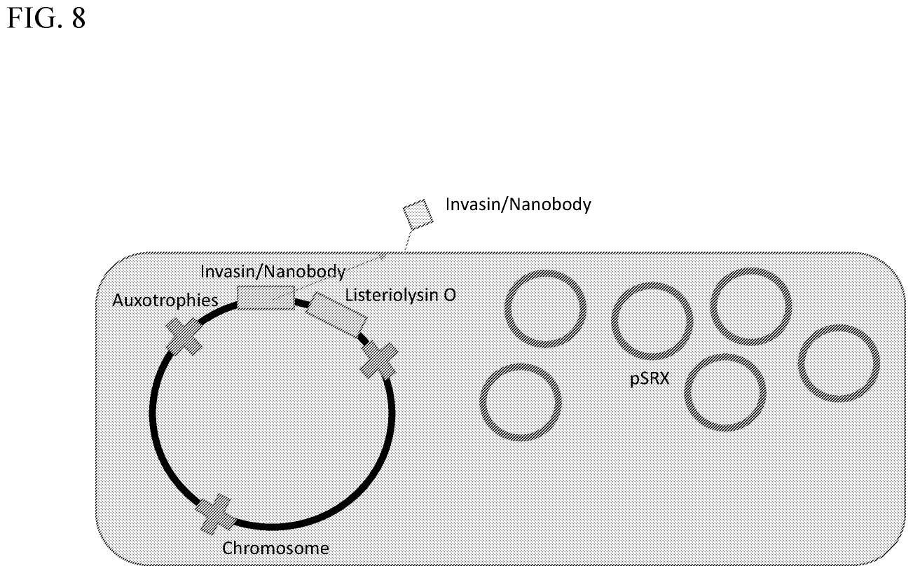

A pMUT1-based plasmid vector having a non-antibiotic selection was constructed. Summarily, E. coli alr gene was used as selection in dapA, alr, dadX triple deletant derivative of E. coli Nissle 1917. GFP gene was cloned into the result plasmid selected using alr. This plasmid was named pSRX. FIG. 8 shows a schematic representation of an embodiment of the genetically engineered bacterium of the present disclosure. This strain is an E. coli Nissle 1917 (EcN) derivative harboring one or more auxotrophic mutation(s) (shown by X), further having genes encoding surface protein and listeriolysin O integrated in the genome. This strain does not contain the plasmid pMUT1, but contains the plasmid pSRX, a pMUT1-based derivative, which is selected using complementation of an auxotrophic mutation as the selection mechanism. Plasmid pSRX also carries a detection marker, which is exemplified herein by GFP.

### Example 2

### In Vitro Detection of Colorectal Carcinoma Cells

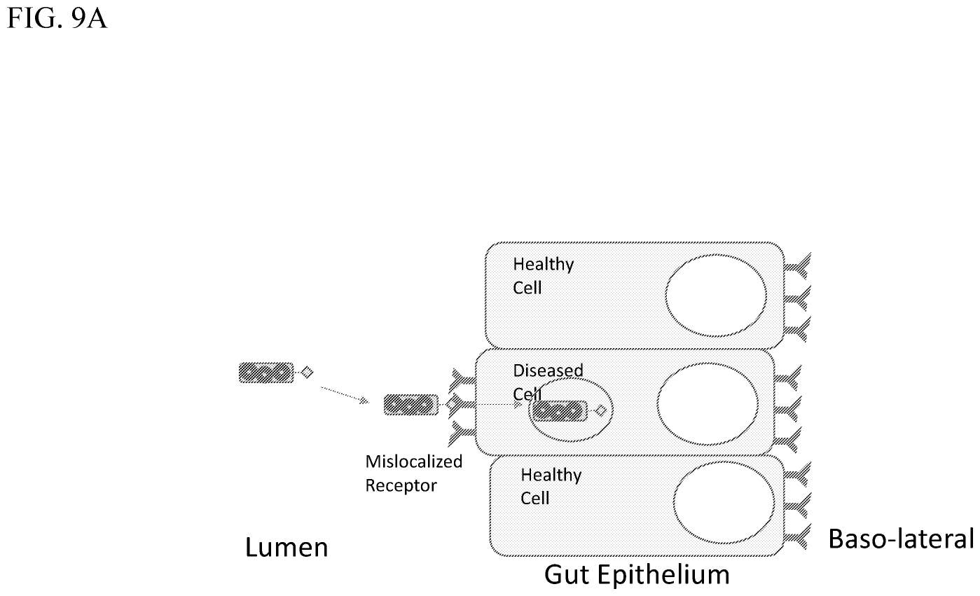

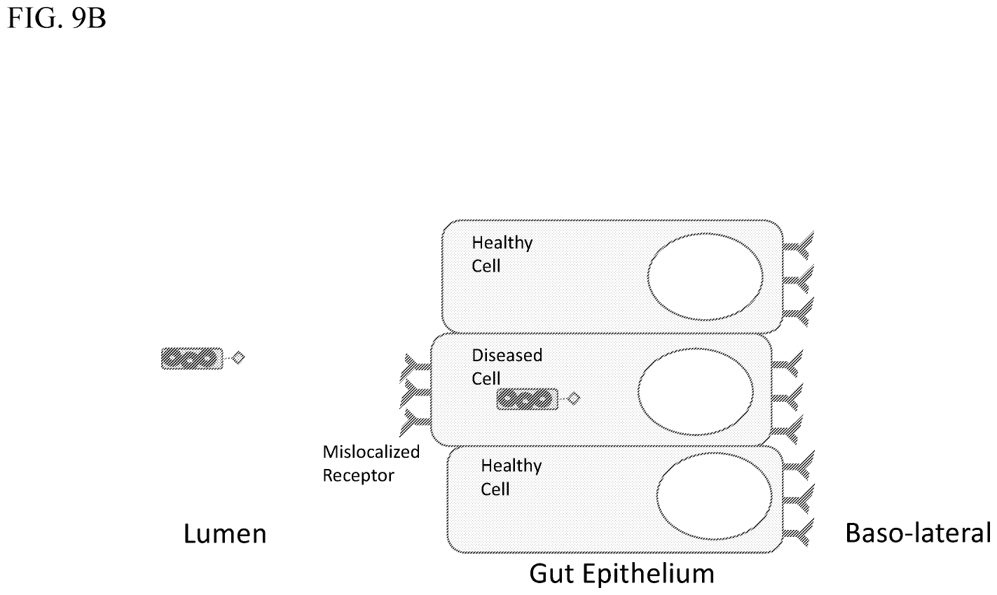

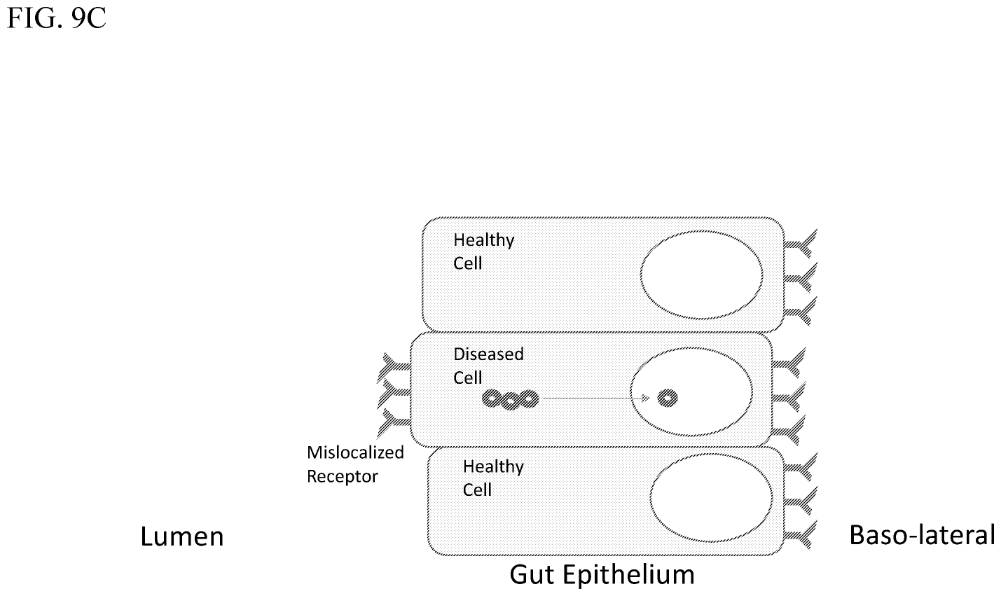

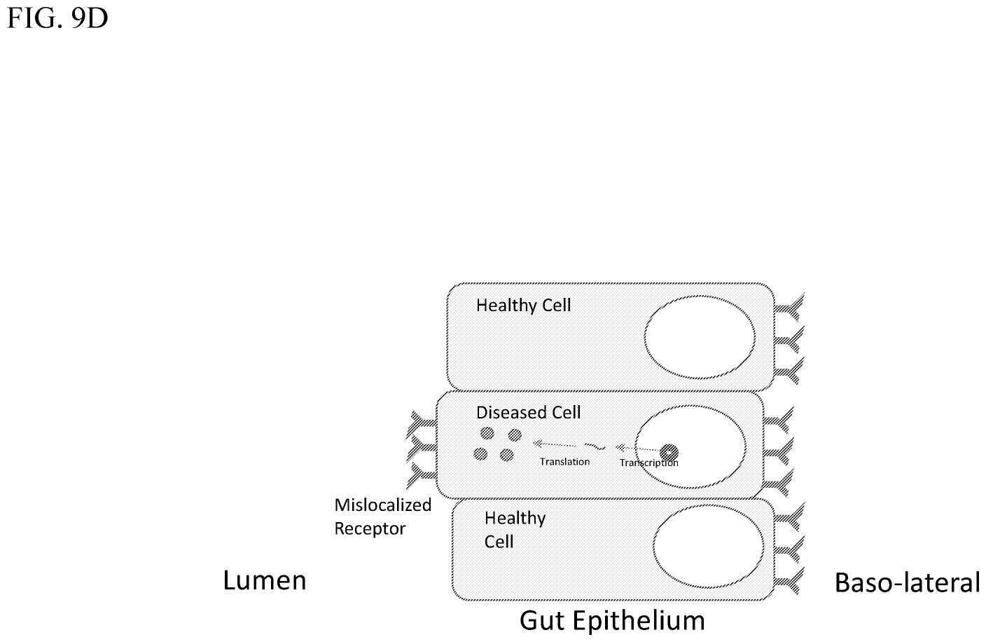

Bacteria of the current disclosure can specifically detect diseased cells. Without being bound by theory, it is hypothesized that detection of diseased cells proceeds through four distinct steps. As shown in FIG. 9A, the genetically engineered microorganisms of the current disclosure bind to diseased epithelial cells through mislocalized receptors, and undergo internalization. Upon internalization, as shown in FIG. 9B, bacteria undergo lysis due to the dapA attenuation mutation, which causes a defect in cell wall synthesis. Listeriolysin O (LLO) is then released and lyses the phagosome or is naturally exported from the E. coli strain. As shown in FIG. 9C, the plasmid carrying detection marker undergoes nuclear localization, optionally guided by binding of a protein that includes a nuclear localization signal. As shown in FIG. 9D, the plasmid carrying detection marker drives the expression of the detection marker in the diseased epithelial cells of GI tract.

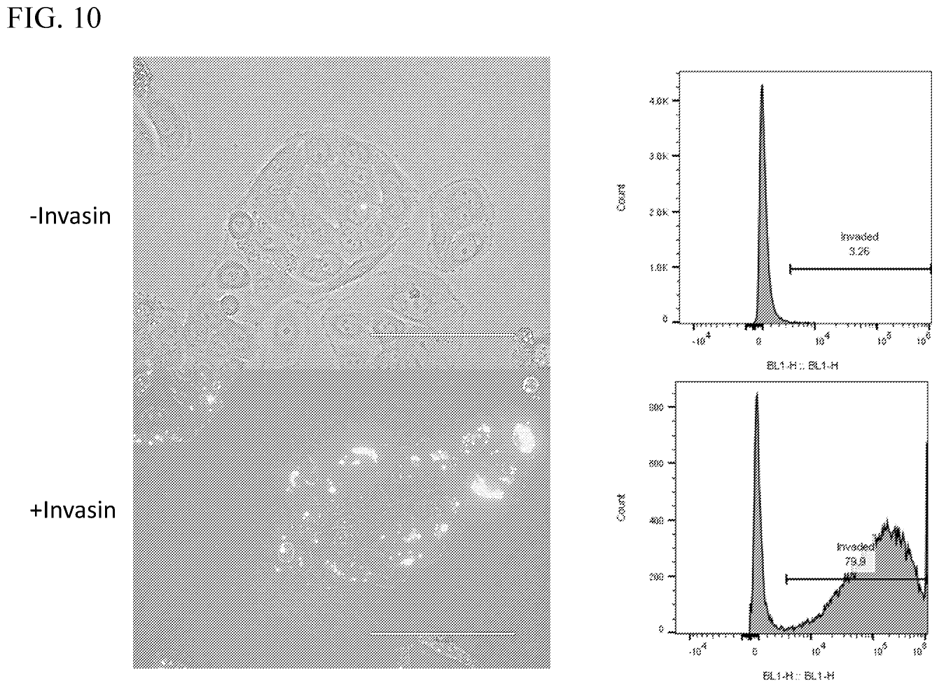

To test this scheme, a strain containing invasion machinery was constructed. The invasion machinery consists of a bacterial surface protein that binds to a protein on the mammalian cell surface and facilitating endocytosis of the bacterium. The initial bacterial surface protein tested was the inv gene from Yersinia pseudotuberculosis coding for the protein invasin. Invasin binds to integrins on the surface of mammalian cells and facilitates endocytotsis. The strain is E. coli Nissle 1917 harboring a pMUT1 derived plasmid that expresses inv under control of the proD constitutive promoter. The plasmid also included a gene encoding a detectable marker (GFP) under the control of a bacterial promoter to make the bacteria easily visible and distinguishable from the mammalian cells. The bacteria from this strain were coincubated with SW480 (colorectal cancer derived cell line) for one hour, followed by washing away of extracellular bacteria. SW480 cells were visualized by fluorescence microscopy, removed from the plate, and then analyzed by flow cytometry to identify the portion of the SW480 cells that were successfully invaded by the bacterial strain. As shown in FIG. 10, SW480 colorectal cancer cells were only invaded by bacterial cells expressing the invasin gene. These results demonstrate that the genetically engineered microorganisms of the current technology can invade cancer cells in vitro, escape the lysosome and express a detectable marker in the cancer cells.

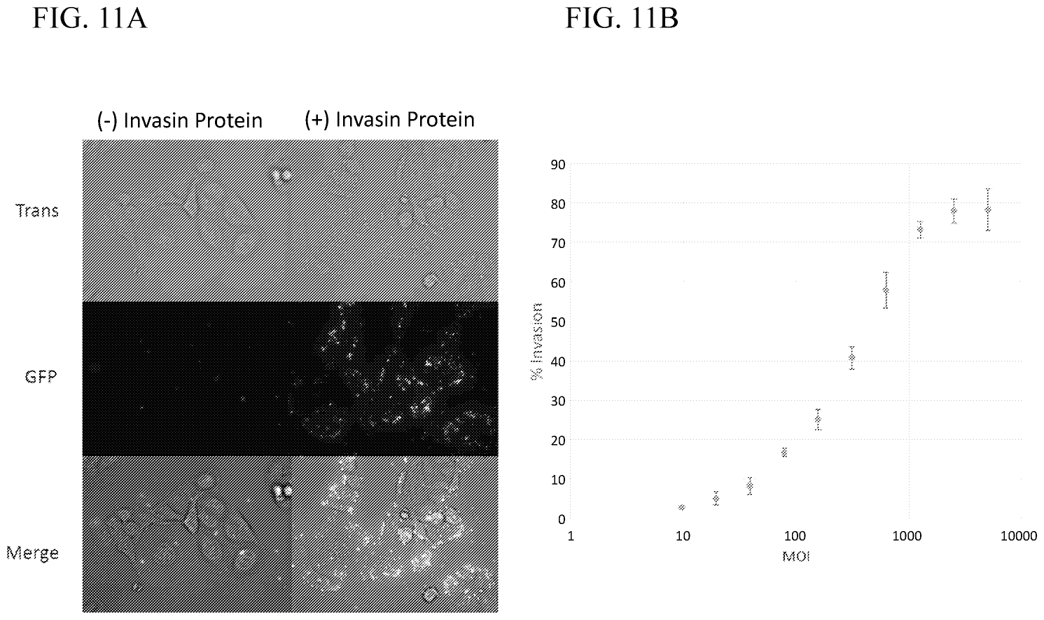

Increasing numbers of the bacteria from the above strain were coincubated with SW480 cells for one hour, followed by washing away of extracellular bacteria. SW480 cells were visualized by fluorescence microscopy and photographed using phase contrast microscopy (“Trans” in FIG. 11A) and fluorescence microscopy (GFP in FIG. 11A). To see which cells are invaded, phase contrast and fluorescence microscopy images were merged. As shown in FIG. 11A, cells contacted with invasin-expressing microorganisms showed staining consistent with invasion of the cells. In contrast, cells treated with invasin⁻ cells did not show such staining. To quantify invasion in mammalian cells, the treated cells were analyzed by flow cytometry to identify the portion of the SW480 cells that were successfully invaded by the bacterial strain. The extent of invasion was plotted as a function of multiplicity of infection (MOI). As shown in FIG. 11B, invasion increased with an increase in MOI and was saturable.

The invasion machinery comprising genes encoding invasin (inv) and listeriolysin O (hly) which allows the bacteria to escape the endocytotic vacuole will be integrated onto the bacterial chromosome at the lambda phage integration site. The integration will occur in such a way as to allow elimination of the antibiotic selection after integration. FIG. 5 shows a schematic representation of this embodiment of the genetically engineered bacterium E. coli Nissle 1917 (EcN) strain. This strain optionally harbors one or more auxotrophic mutation(s) such as dapAΔ, alrΔ, and dadXΔ (shown by X).

### Example 3

### Use of Intimin Scaffold for Display of Cancer-Specific Ligands

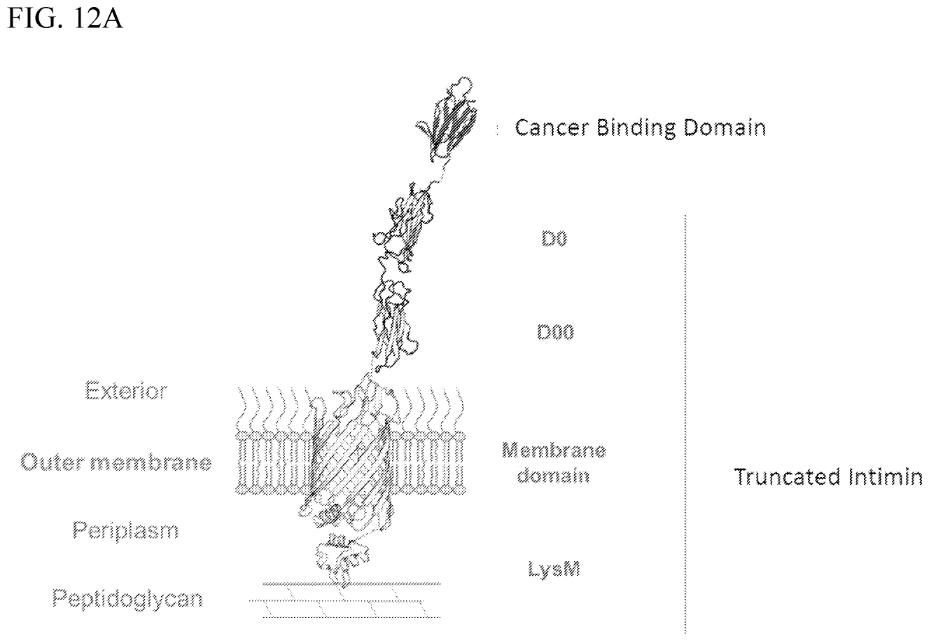

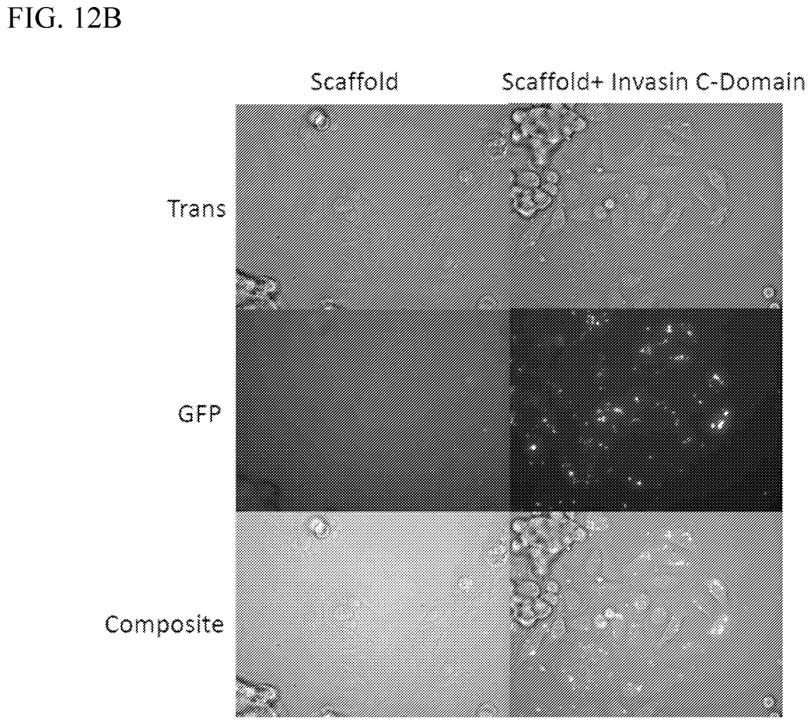

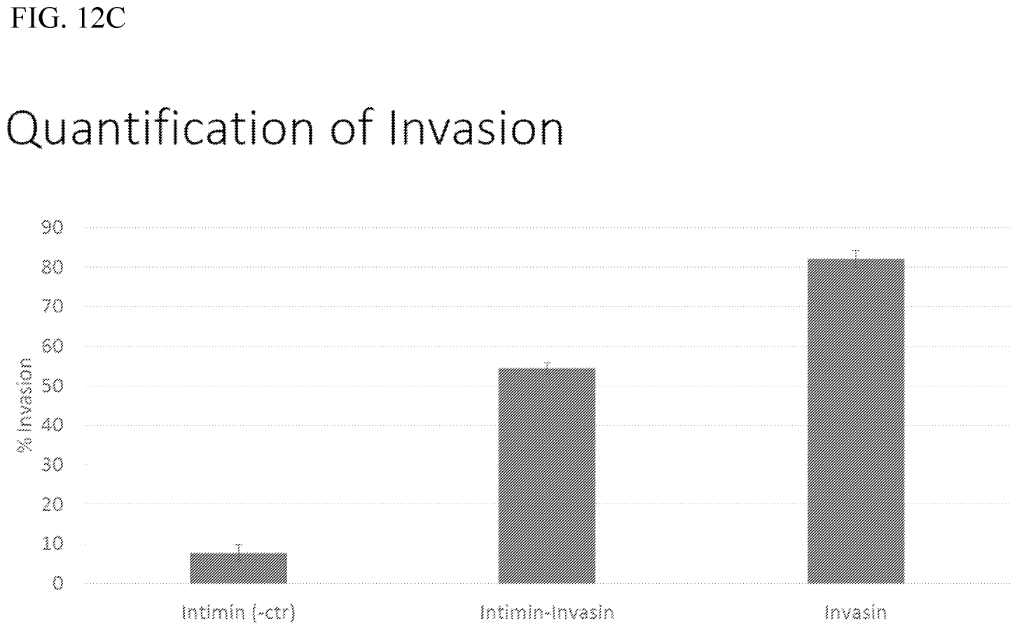

Intimins are proteins from “attaching and effacing” (A/E) pathogens such as enterohemorrhagic Escherichia coli (EHEC) and of Gram-negative bacteria. Intimins play a role in the pathogenicity of the A/E pathogens by promoting tight adhesion to epithelial cells. To evaluate whether intimin may be used to display cancer-specific ligands, a fusion protein of intimin-invasin was made by replacing the three C-terminal domains of intimin (D1, D2 and D3) with C-terminal domain of invasin (FIG. 12A). See Weikum et al., The extracellular juncture domains in the intimin passenger adopt a constitutively extended conformation inducing restraints to its sphere of action, Scientific Reports 10: 21249 (2020). E. coli Nissle 1917 derivative strains expressing an intimin scaffold alone (SEQ ID NO: 3), and the intimin-invasin fusion protein (SEQ ID NO: 4) were created. These strains or the E. coli Nissle 1917 derivative strain expressing invasin were coincubated with human colorectal cancer cells for one hour, followed by washing away of extracellular bacteria. Cancer cells were visualized by fluorescence microscopy and photographed using phase contrast and fluorescence microscopy. To see which cells are invaded, phase contrast and fluorescence microscopy images were merged. As shown in FIG. 12B, cells contacted with bacteria expressing the invasin-intimin fusion protein but not the intimin scaffold exhibited staining consistent with invasion of the cells. To quantitate invasion in mammalian cells, the treated cells were quantitate the fraction of the SW480 cells that were successfully invaded by the bacterial strain. As shown in FIG. 12C, the bacteria expressing an intimin-invasin fusion protein invaded the cancer cells.

These results demonstrate that the intimin scaffold disclosed herein may be used for displaying ligands for specific recognition of cells recognized by the ligands. For instance, cancer-specific ligands may be used for the detection of cancer cells.

### Example 4

### In Vivo Detection of Cancer Cells by the Engineered Microorgamisms

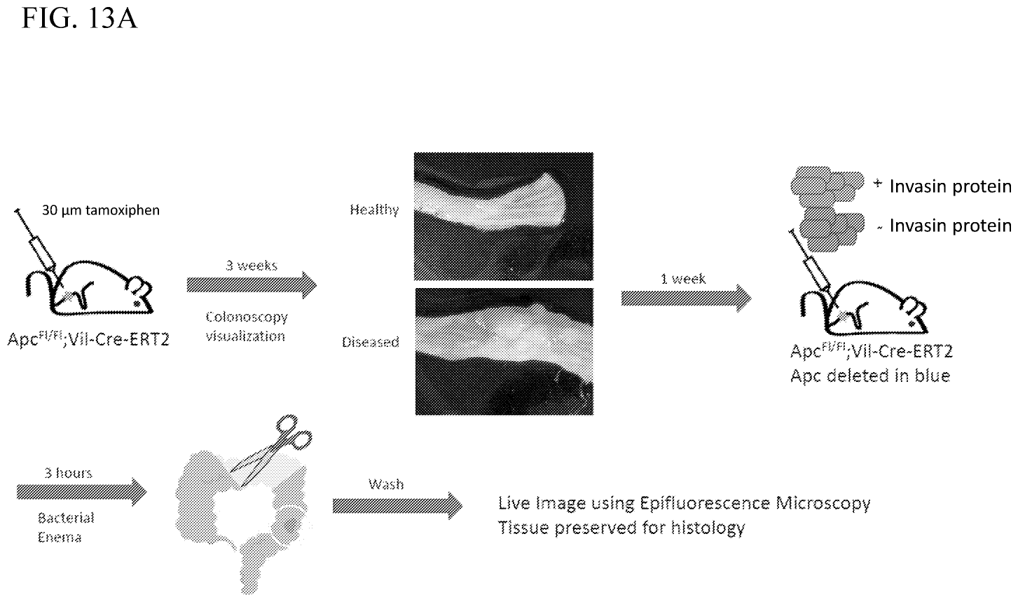

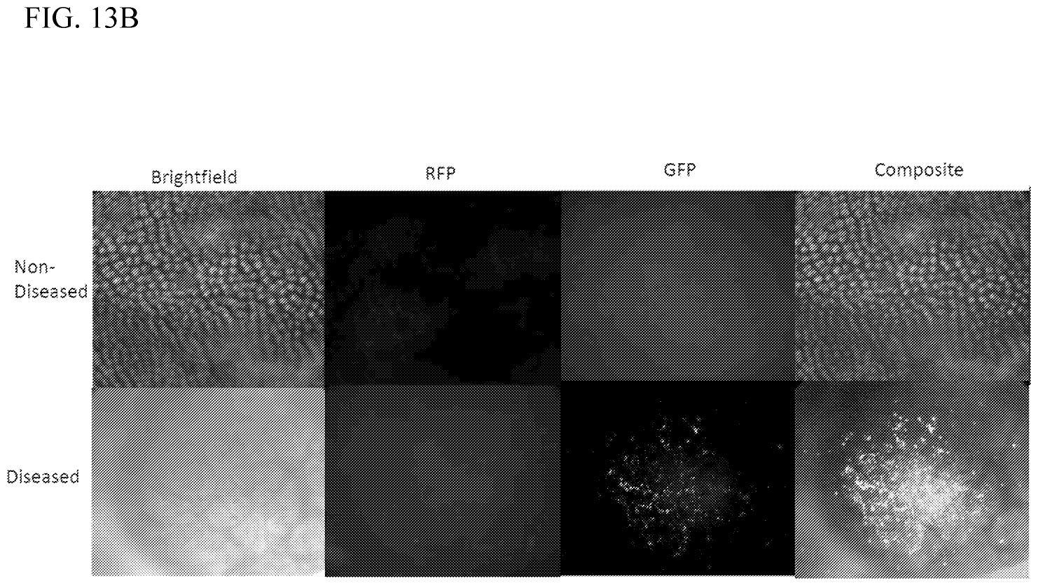

An E. coli Nissle 1917 derivative strain expressing invasin and harboring a plasmid carrying a GFP gene (mNeonGreen) under control of a bacterial promoter (such as a proD promoter) was constructed. A similar strain lacking invasin gene and expressing a RFP gene (mScarlet) was also generated. Cancer cells were detected in vivo using the process as shown in FIG. 13A. Briefly, Apc^{F1/F1}; Vil-Cre-ERT2 mice were administered 30 μM 4—OH tamoxifen to induce carcinogenesis. Tumors were visualized by colonoscopy. A mixture of bacteria expressing invasin and harboring the GFP expression vector and the bacteria not expressing invasin but harboring the RFP expression vector were administered to the mice using an enema. After 3 hours, mice were sacrificed, colons were excised, washed and observed using epifluorescence microscopy and brightfield microscopy (FIG. 13A).

As expected, the tumors from the mice treated with the bacterial mixture showed a background level of fluorescence of both GFP and RFP at the proximal (i.e. non-diseased) portion of the colon (FIG. 13B). On the other hand, as shown in FIG. 13B, the tumors at the distal end of the colon from the mice treated with the bacterial mixture showed clear invasion in the diseased tissue of only the bacteria expressing invasin and GFP. A merge of GFP fluorescence and brightfield images showed that the GFP fluorescence was detected in the areas corresponding to the tumor.

These results demonstrate that the genetically engineered microorganisms disclosed herein can discriminate between diseased vs. non diseased tissue in vivo. Accordingly, genetically engineered microorganisms of the present disclosure are useful in methods of detecting cancer lesions of the gastrointestinal tract.

### Example 5

### The Requirements for Efficient Delivery of DNA Payloads

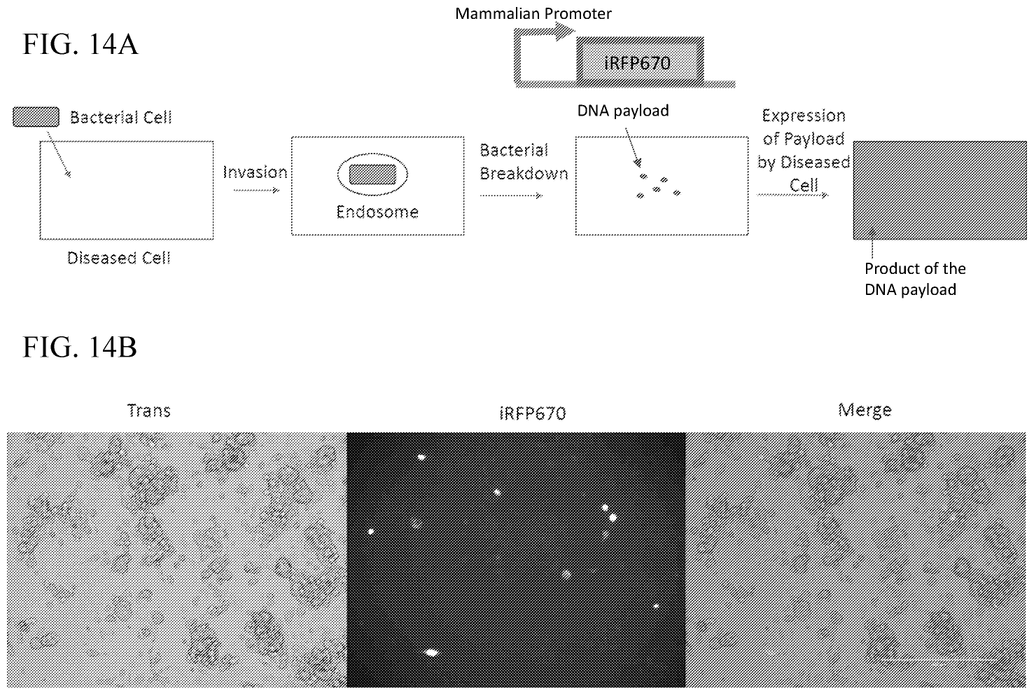

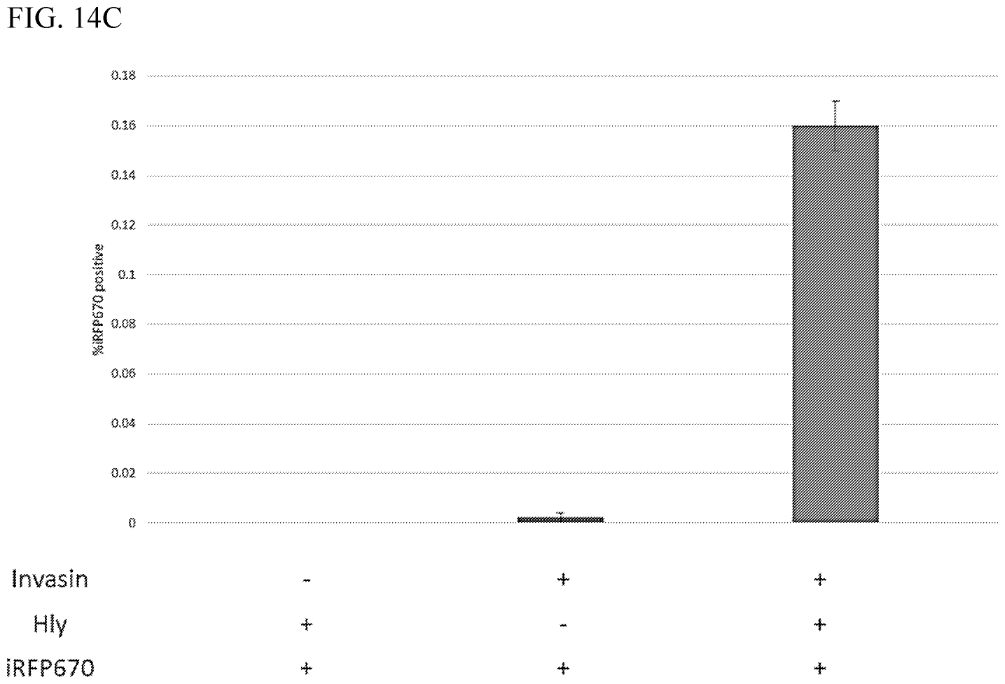

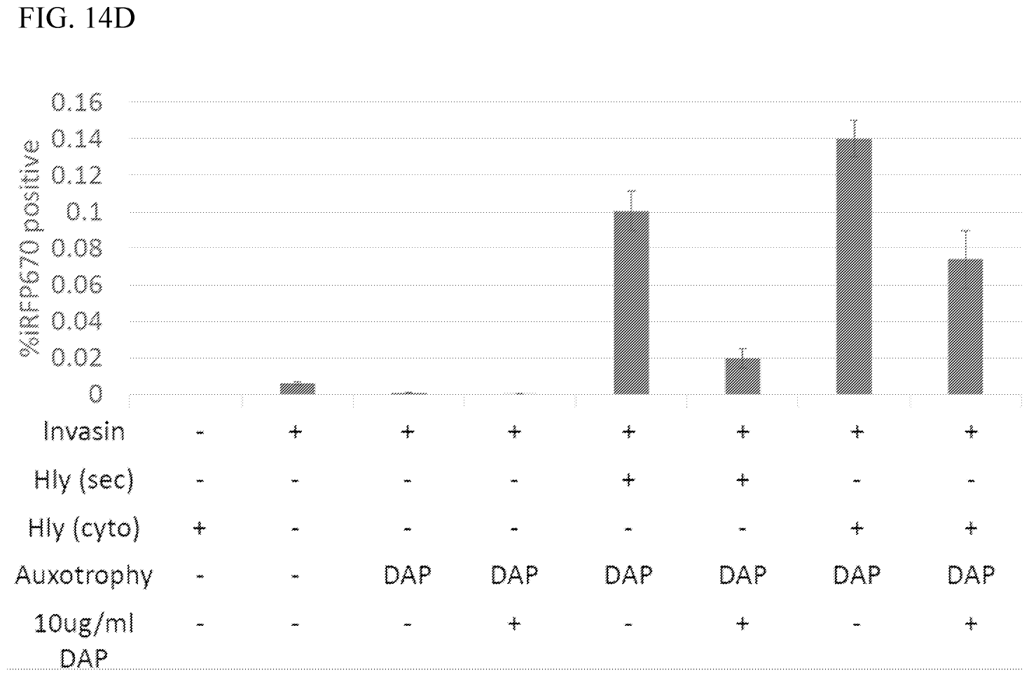

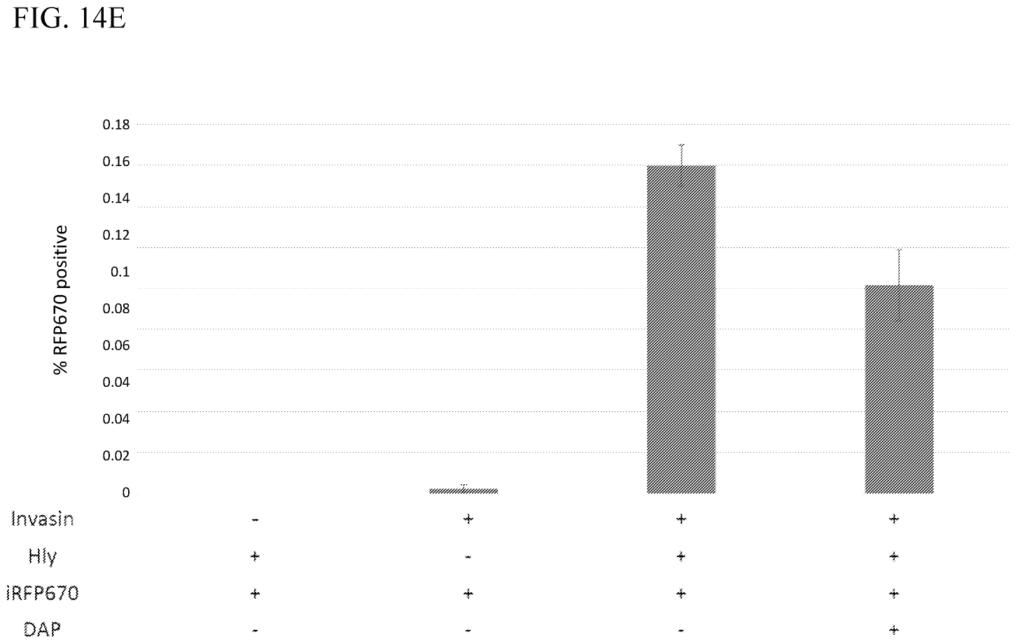

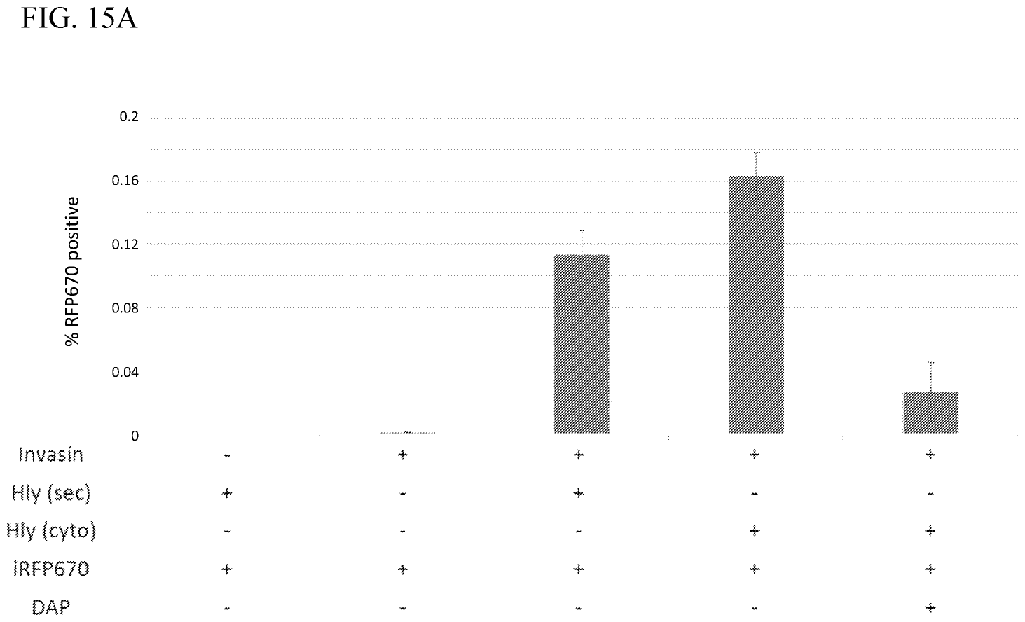

A base strain to study delivery of DNA payloads was an E. coli Nissle 1917 dapAΔ alrΔ dadXA strain harboring a plasmid comprising invasin controlled by bacterial a promoter and another multicopy plasmid harboring listeriolysin O (Hly; SEQ ID NO: 2) controlled by a bacterial promoter and an iRFP670 gene (SEQ ID NO: 5) controlled by a mammalian promoter (CMV promoter). The proposed mechanism of delivery of DNA payloads, without being bound by theory, is shown in FIG. 14A. To assay the delivery of DNA payloads, the base strain was coincubated with human cancer cells for 3 hours, followed by washing away of extracellular bacteria. Cancer cells were visualized by fluorescence microscopy and photographed using phase contrast and fluorescence microscopy. To visualize the cells that express the iRFP670 gene (SEQ ID NO: 5) controlled by the mammalian promoter (CMV promoter), phase contrast and fluorescence microscopy images were merged. As shown in FIG. 14B, RFP fluorescence was seen in many cells. Since the iRFP670 gene was controlled by a mammalian promoter (CMV promoter), moreover, iRFP670 requires biliverdin to fluoresce which the bacteria don't make. Therefore, these data provide evidence that the microorganisms disclosed herein must have transferred DNA to the nucleus of cancer cells.

To evaluate the requirement for invasin and listeriolysin O, the following strains were constructed: (1) an E. coli Nissle 1917 dapAΔ alrΔ dadXΔ strain harboring a plasmid comprising listeriolysin O (Hly; SEQ ID NO: 2) under control of a bacterial promoter and the iRFP670 gene (SEQ ID NO: 5) under the control of a mammalian promoter (CMV promoter) and another plasmid harboring the intimin scaffold (SEQ ID NO: 3) expressed from a bacterial promoter(the invasin⁻ strain); (2) E. coli Nissle 1917 dapAΔ alrΔ dadXΔ strain harboring a plasmid comprising invasin (SEQ ID NO: 1) under the control of a bacterial promoter and another plasmid harboring the iRFP670 gene (SEQ ID NO: 5) under the control of a mammalian promoter (the listeriolysin O⁻ strain); and 3) an E. coli Nissle 1917 dapAΔ alrΔ dadXΔ strain harboring a plasmid comprising listeriolysin O (Hly) under control of a bacterial promoter and the iRFP670 gene (SEQ ID NO: 5) under the control of a mammalian promoter and another plasmid harboring the invasin gene expressed from a bacterial promoter (the test strain).

To assay the delivery of DNA payloads, human cancer cells were coincubated with the test strain, the listeriolysin O⁻ strain or the invasin⁻ strain for one hour, followed by washing away of extracellular bacteria. To quantitate the delivery of DNA payloads, the cells were analyzed by flow cytometry to identify the portion of the cancer cells that were successfully expressed the iRFP670 gene (SEQ ID NO: 5) under a mammalian promoter. As shown in FIG. 14C, shows the listeriolysin O⁻ and the invasin⁻ strains were deficient in delivery of DNA payloads. The base strain which contained both invasin and listeriolysin O was capable of delivery of DNA payloads (FIG. 14C). These results demonstrate that both the surface protein (without limitation, e.g., invasin or invasin-intimin fusion protein) and lysin (without limitation, e.g., listeriolysin O that is secreted or retained in the cytoplasm) are required for efficient delivery of DNA payloads.

Then, the following strain capable of secreting listeriolysin O was constructed: an E. coli Nissle 1917 dapAΔ alrΔ dadXΔ strain harboring a plasmid comprising a modified secretory listeriolysin O (Hly) gene, the two additional genes required to form the machinery to secretion of the listeriolysin O gene (HlyB and HlyD) under a bacterial promoter and the iRFP670 gene (SEQ ID NO: 5) under the mammalian promoter and an additional plasmid harboring the invasin gene under a bacterial promoter (the listeriolysin O-secreting strain). Similarly, an E. coli Nissle 1917 dapA⁺ alrΔ dadXΔ strain harboring a plasmid comprising listeriolysin O (Hly) under a bacterial promoter and an additional plasmid containing the intimin scaffold (the dapA⁺ invasin⁻ strain) and another E. coli Nissle 1917 dapA⁺ alrΔ dadXΔ strain harboring a plasmid comprising invasin gene under a bacterial promoter and an additional plasmid harboring the iRFP670 gene under a mammalian promoter (the dapA⁺ listeriolysin O⁻ strain) was constructed.

To assay the delivery of DNA payloads, human cancer cells were coincubated with the dapA⁺ listeriolysin O⁻ strain, the dapA⁺ invasin⁻ strain, the invasin⁻ strain, the listeriolysin O-secreting strain or the base strain for one hour, followed by washing away of extracellular bacteria. The latter three incubations carried out in duplicate, with and without 10 μg/ml diamino pimelic acid (DAP). To quantitate the delivery of DNA payloads, the cells were analyzed by flow cytometry. As expected, the dapA⁺ listeriolysin O⁻ strain, the dapA⁺ invasin⁻ strain did not exhibit an iRFP670 signal, indicating an inability to deliver of DNA payloads (FIG. 14D). The invasin⁻ strain did not produce an iRFP670 signal, indicating an inability to deliver the DNA payloads (FIG. 14D). Both the listeriolysin O-secreting strain and the base strain produced iRFP670⁺ cells, showing a proficiency to deliver of DNA payloads (FIG. 14D). Interestingly, as shown in FIG. 14D, the addition of Dap to culture media decreases the extent of the delivery of DNA payloads (see also FIG. 14E).

These results indicate, without being bound by theory, that bacterial lysis upon invasion may be required for efficient delivery of DNA payloads by the engineered microbial strains disclosed herein.

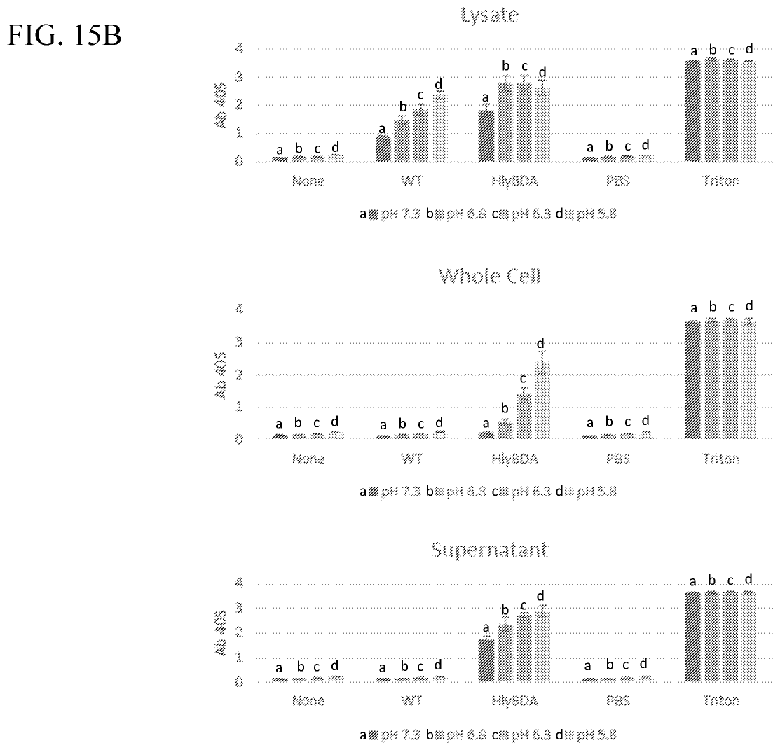

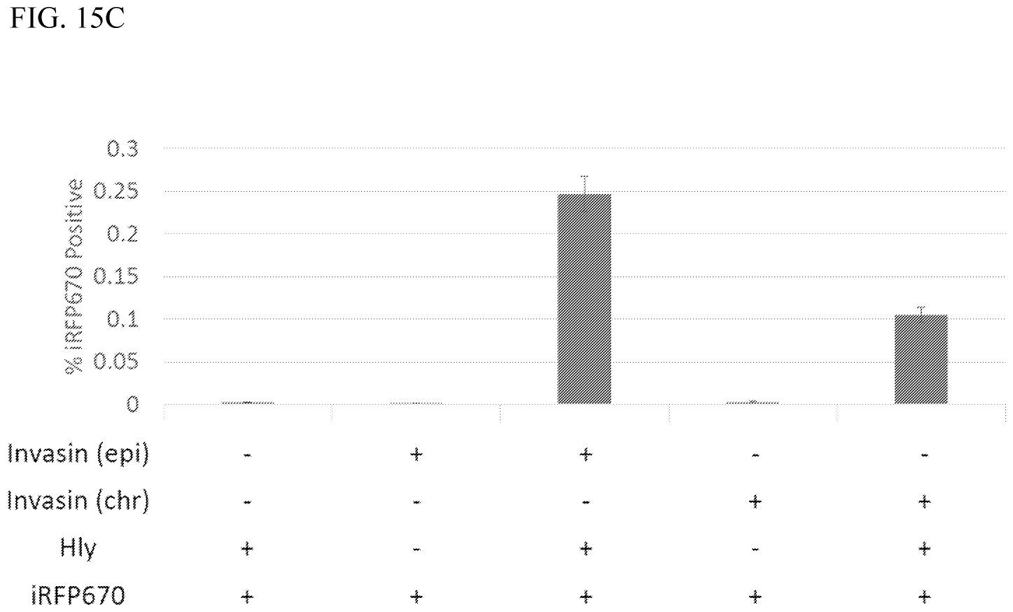

The secretion of listeriolysin O was confirmed. Briefly, the listeriolysin O-secreting strain and the base strain were grown to mid-log phase. An aliquot of each of the strains was recovered for assay of listeriolysin O activity of whole cells. Another aliquot of each of the strains was spun down and culture supernatant was recovered. Cell pellets were washed with PBS and sonicated to disrupt cell membranes and to prepare bacterial lysates. The whole cell samples, supernatants and the bacterial lysate samples were incubated with RBCs at pH 7.3, 6.8, 6.3 or 5.8. Untreated RBCs, PBS-treated RBCs were used as negative controls, and triton-treated RBCs were used as positive controls for hemolysis. The treated RBC samples were centrifuged and the absorbance of supernatants at 405 nm was measured to assess hemolysis. As expected, the untreated RBCs, PBS-treated RBCs showed a background level of hemolysis, and triton-treated RBCs showed pH-independent hemolysis (FIG. 15B). As shown in FIG. 15B, the lysates of both the listeriolysin O-secreting strain and the base strain showed pH-dependent hemolysis consistent with what is expected for listeriolysin O. Further, culture supernatants of the listeriolysin O-secreting strain but not the base strain produced a pH-dependent hemolysis at low pH (FIG. 15B). These results indicate that the listeriolysin O-secreting strain secretes listeriolysin O.

To test the effect of secretion of listeriolysin O on the delivery of DNA payloads, human cancer cells were coincubated with the invasin⁻ strain, the listeriolysin O⁻ strain, the listeriolysin O-secreting strain or the base strain for one hour, followed by washing away of extracellular bacteria. The delivery of DNA payloads was analyzed by flow cytometry. As shown in FIG. 15A, the listeriolysin O-secreting strain and the base strain were both proficient at delivering DNA payloads.

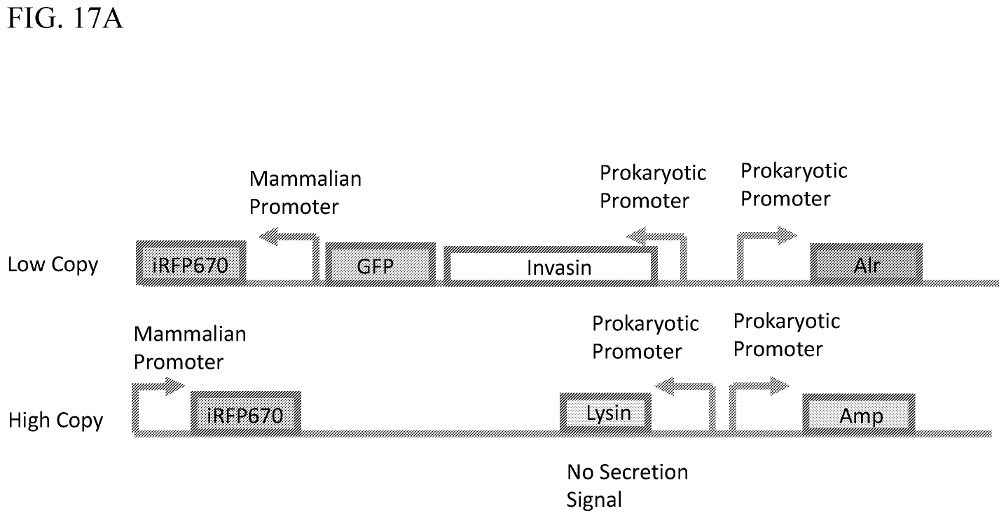

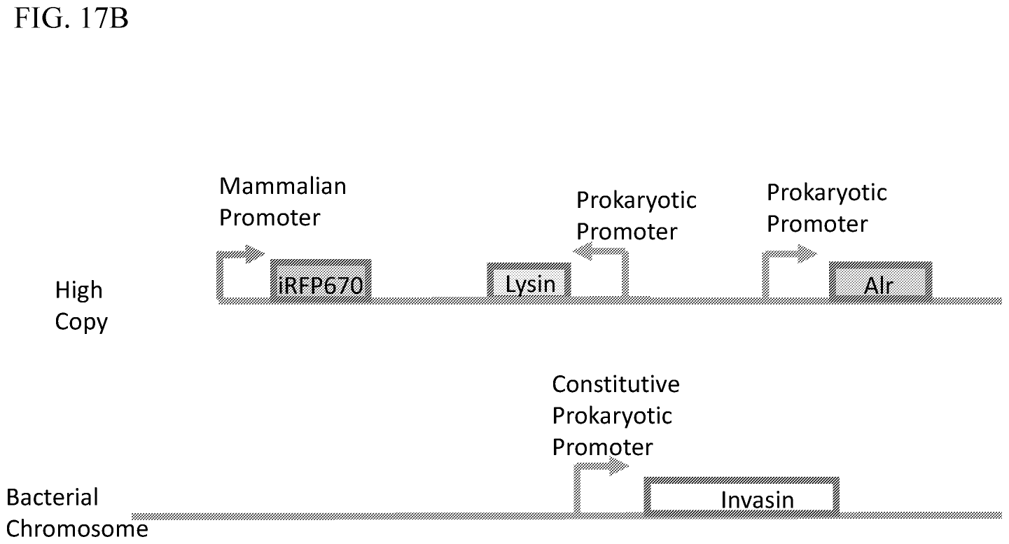

To test whether the invasin gene may be integrated on the bacterial chromosome for efficient DNA delivery, an invasin integrant strain was constructed: an E. coli Nissle 1917 dapAΔ alrΔ dadXΔ strain harboring a plasmid comprising the listeriolysin O (Hly) gene under the control of a bacterial promoter and the iRFP670 gene under the control of a mammalian promoter. This strain included invasin gene under control of a constitutive prokaryotic promoter integrated at the lambda attB site on the bacterial chromosome. The exemplary organization of such a strain is shown in FIG. 17B. An invasin integrant listeriolysin O⁻ was also constructed.

To test the effect of integration of invasin gene on the delivery of DNA payloads, human cancer cells were coincubated with listeriolysin O⁻ strain, the invasin⁻ strain, the invasin integrant strain, invasin integrant listeriolysin O⁻ strain, or the base strain. Extracellular bacteria were washed away, and the delivery of DNA payloads was analyzed by flow cytometry. As shown in FIG. 15C, the invasin integrant strain and the base strain were both proficient of delivering DNA payloads. As expected, the delivery of DNA payloads was not observed when either invasin (SEQ ID NO: 1) or listeriolysin O (SEQ ID NO: 2) were absent from the strains (FIG. 15C). These data indicated that the integration of invasin gene into the bacterial chromosome is a viable approach for constructing the strains disclosed herein.

### Example 6

### The Delivery of mRNA Payloads

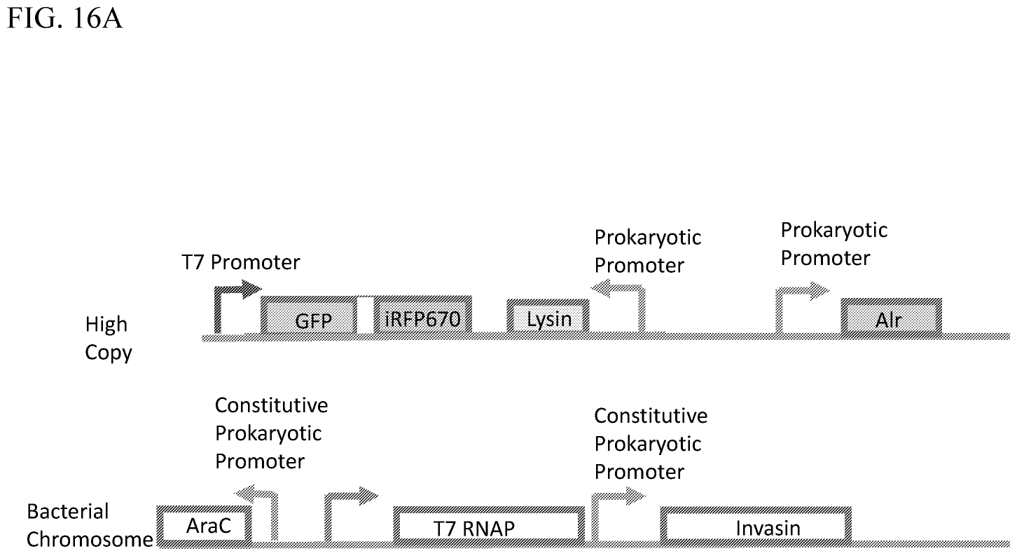

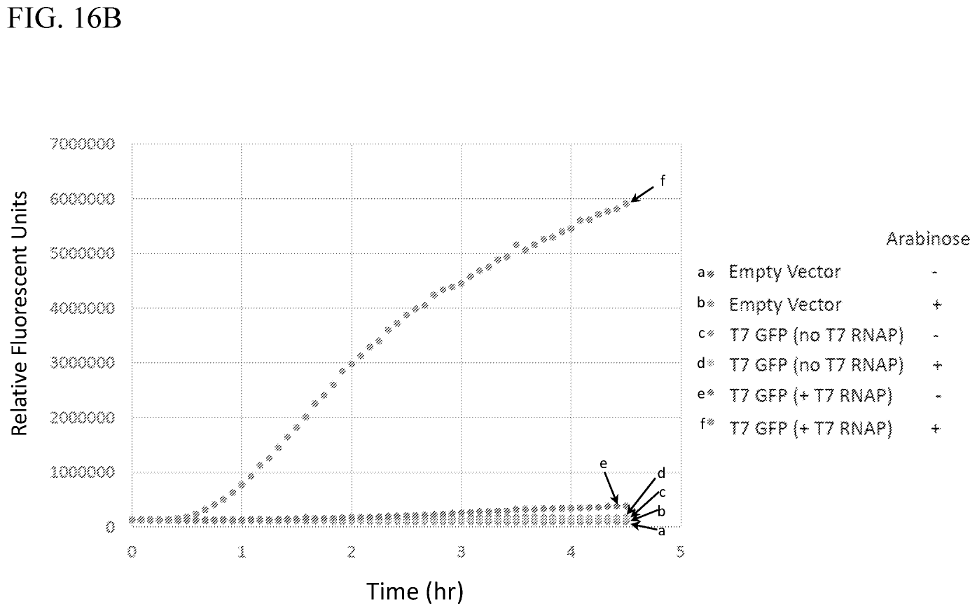

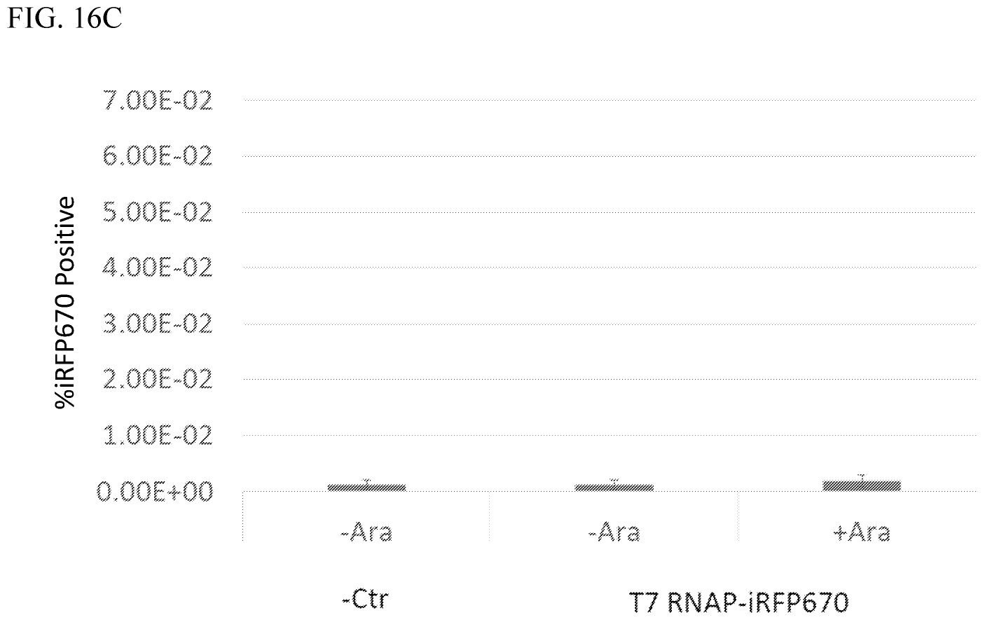

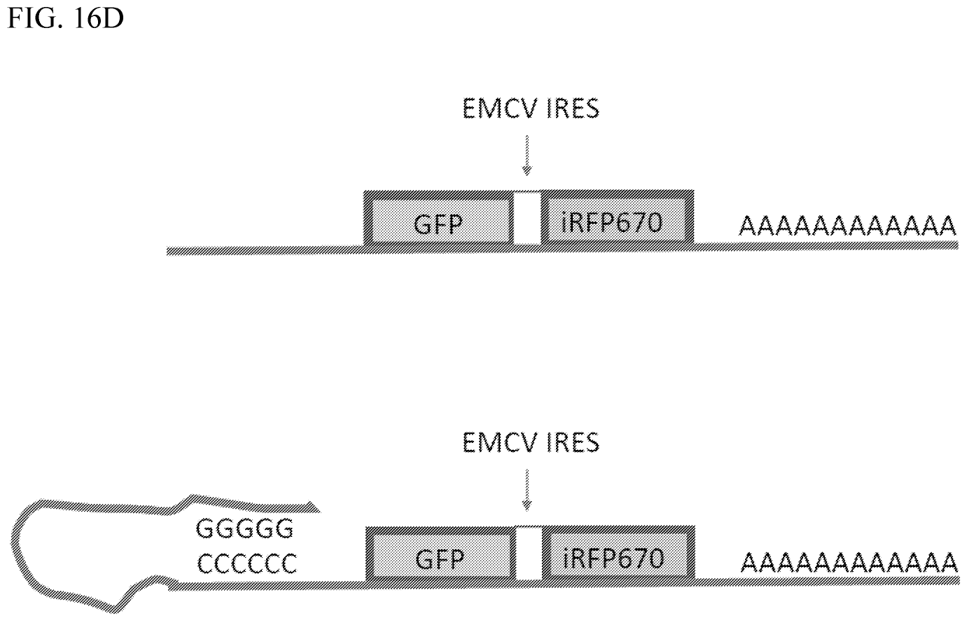

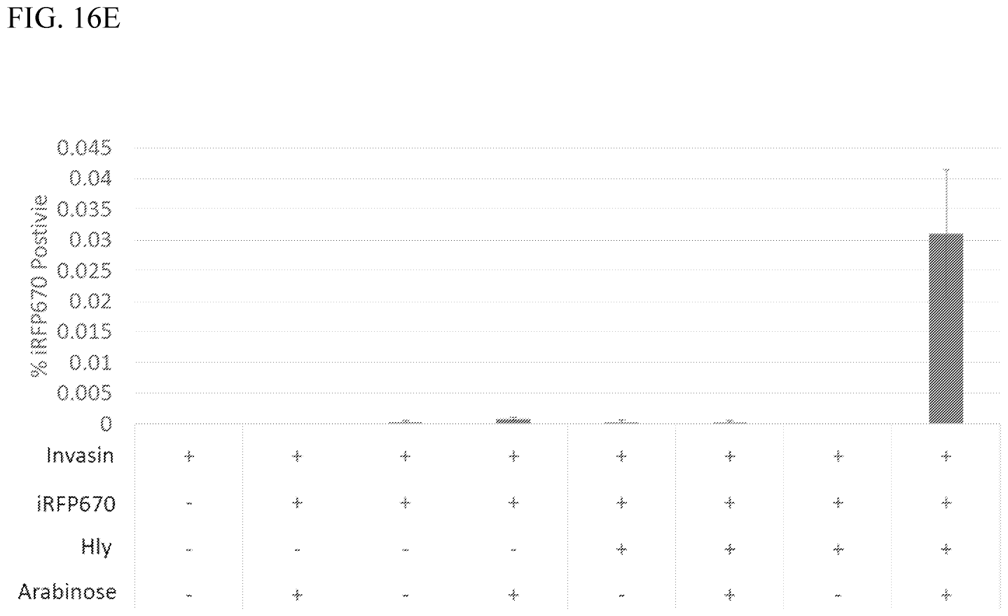

Next, strains for RNA delivery were constructed. Towards that, a base strain for RNA delivery was constructed (FIG. 16A). This strain had the gene encoding T7 RNA polymerase controlled by the araBAD promoter and invasin controlled by a constitutive bacterial promoter integrated in the bacterial chromosome. To construct such a strain, a cassette containing araC, T7 RNA polymerase gene under an araBAD promoter, and invasin gene under a constitutive E. coli promoter were incorporated in a lambda-based integration vector. An integrant harboring integration of the cassette was selected (FIG. 16A). The strain was further transformed with a plasmid harboring the listeriolysin O gene (lysin in FIG. 16A), a GFP-EMCV IRES-iRFP670 cassette controlled by a T7 promoter on a high copy plasmid selected by complementation of alrΔ dadXΔ by an alr gene controlled by its native promoter (FIG. 16A). Control strains lacking the GFP-EMCV IRES-iRFP670 cassette or lacking the gene encoding T7 RNA polymerase were also constructed.

The GFP is made by the bacteria and acts as both a visible marker for invasion and a confirmation of successful induction of RNA production from the T7 promoter. The expression of iRFP670, which is translated in mammalian cells, serves as a marker of mRNA delivery. The control strains and the base strain were grown in the absence or presence of arabinose and relative GFP expression was measured by spectrophotometry. As shown in FIG. 16B, the base strain exhibited the production of the mRNA cassette when induced with arabinose. In contrast, the base strain grown without arabinose or the control strains lacking either the GFP-EMCV IRES-iRFP670 cassette or lacking the gene encoding T7 RNA polymerase did not show detectable expression of GFP (FIG. 16B).

These results demonstrate that T7 RNAP can be induced for RNA production in E. coli Nissle 1917.

To assay the delivery of RNA payloads, human cancer cells were coincubated with the control strain lacking the gene encoding T7 RNA polymerase or the base strain for one hour, followed by washing away of extracellular bacteria. To quantitate the delivery of RNA payloads, the cells were analyzed by flow cytometry. As expected, the control strain lacking the gene encoding T7 RNA polymerase showed a background level of expression of iRFP670 (FIG. 16C). However, the base strain also showed a background level of expression of iRFP670 indicating the base strain was unable to deliver RNA payloads (FIG. 16C).

Without being bound by theory, it was hypothesized that the inability to deliver of RNA payloads may be because of instability of mRNA. Therefore, to stabilize mRNA, a stable hairpin was introduced at 5′ end of the mRNA, as shown in FIG. 16D. To assay the delivery of RNA payloads, human cancer cells were coincubated with the control strain lacking the GFP-EMCV IRES-iRFP670 cassette and listeriolysin O (Hly), the control strain lacking listeriolysin O (Hly), the base strain without a 5′ hairpin in the GFP-EMCV IRES-iRFP670 cassette, or the base strain having a 5′ hairpin in the GFP-EMCV IRES-iRFP670 cassette. After incubation for one hour, extracellular bacteria were washed away and the cancer cells were analyzed by flow cytometry. As expected, the control strains showed a background level of expression of iRFP670 (FIG. 16E). The base strain lacking the 5′ hairpin in the GFP-EMCV IRES-iRFP670 cassette also showed a background level of expression of iRFP670 (FIG. 16C). In contrast, the base strain having the 5′ hairpin in the GFP-EMCV IRES-iRFP670 cassette exhibited cells expressing iRFP670.

These results demonstrate that the bacterial strains disclosed herein are capable of delivering RNA to cancer cells.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present invention is not entitled to antedate such publication by virtue of prior invention.

As used herein, all headings are simply for organization and are not intended to limit the disclosure in any manner. The content of any individual section may be equally applicable to all sections.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Sequences of Surface Proteins (Invasin and Analogs)
SEQ ID NO: 1
Amino acid sequence of Invasin
MVFQPISEFLLIRNAGMSMYFNKIISFNIISRIVICIFLICGMFMAGASEKYDANAPQQVQPYS
VSSSAFENLHPNNEMESSINPFSASDTERNAAIIDRANKEQETEAVNKMISTGARLAASGRASD
VAHSMVGDAVNQEIKQWLNRFGTAQVNLNFDKNFSLKESSLDWLAPWYDSASFLFFSQLGIRNK
DSRNTLNLGVGIRTLENGWLYGLNTFYDNDLTGHNHRIGLGAEAWTDYLQLAANGYFRLNGWHS
SRDFSDYKERPATGGDLRANAYLPALPQLGGKLMYEQYTGERVALFGKDNLQRNPYAVTAGINY
TPVPLLTVGVDQRMGKSSKHETQWNLQMNYRLGESFQSQLSPSAVAGTRLLAESRYNLVDRNNN
IVLEYQKQQVVKLTLSPATISGLPGQVYQVNAQVQGASAVREIVWSDAELIAAGGTLTPLSTTQ
FNLVLPPYKRTAQVSRVTDDLTANFYSLSALAVDHQGNRSNSFTLSVTVQQPQLTLTAAVIGDG
APANGKTAITVEFTVADFEGKPLAGQEVVITTNNGALPNKITEKTDANGVARIALTNTTDGVTV
VTAEVEGQRQSVDTHFVKGTIAADKSTLAAVPTSIIADGLMASTITLELKDTYGDPQAGANVAF
DTTLGNMGVITDHNDGTYSAPLTSTTLGVATVTVKVDGAAFSVPSVTVNFTADPIPDAGRSSFT
VSTPDILADGTMSSTLSFVPVDKNGHFISGMQGLSFTQNGVPVSISPITEQPDSYTATVVGNTA
GDVTITPQVDTLILSTLQKKISLFPVPTLTGILVNGQNFATDKGFPKTIFKNATFQLQMDNDVA
NNTQYEWSSSFTPNVSVNDQGQVTITYQTYSEVAVTAKSKKFPSYSVSYRFYPNRWIYDGGTSL
VSSLEASRQCQGSDMSAVLESSRATNGTRAPDGTLWGEWGSLTAYSSDWQSGEYWVKKTSTDFE
TMNMDTGALVQGPAYLAFPLCALAI
SEQ ID NO: 3
Amino acid sequence of Intimin Scaffold
MITHGCYTRTRHKHKLKKTLIMLSAGLGLFFYVNQNS FANGENYFKLGSDSKLLTHDSYQNRLF
YTLKTGETVADLSKSQDINLSTIWSLNKHLYSSESEMMKAAPGQQIILPLKKLPFEYSALPLLG
SAPLVAAGGVAGHTNKLTKMSPDVTKSNMTDDKALNYAAQQAASLGSQLQSRSLNGDYAKDTAL
GIAGNQASSQLQAWLQHYGTAEVNLQSGNNFDGSSLDFLLPFYDSEKMLAFGQVGARYIDSRFT
ANLGAGQRFFLPANMLGYNVFIDQDFSGDNTRLGIGGEYWRDYFKSSVNGYFRMSGWHESYNKK
DYDERPANGFDIRFNGYLPSYPALGAKLIYEQYYGDNVALFNSDKLQSNPGAATVGVNYTPIPL
VTMGIDYRHGTGNENDLLYSMQFRYQFDKSWSQQIEPQYVNELRTLSGSRYDLVQRNNNIILEY
KKQDILSLNIPHDINGTEHSTQKIQLIVKSKYGLDRIVWDDSALRSQGGQIQHSGSQSAQDYQA
ILPAYVQGGSNIYKVTARAYDRNGNSSNNVQLTITVLSNGQVVDQVGVTDFTADKTSAKADNAD
TITYTATVKKNGVAQANVPVSFNIVSGTATLGANSAKTDANGKATVTLKSSTPGQVVVSAKTAE
MTSALNASAVIFFDGATR
SEQ ID NO: 4
Amino acid sequence of Intimin-Invasin fusion protein
MITHGCYTRTRHKHKLKKTLIMLSAGLGLFFYVNQNSFANGENYFKLGSDSKLLTHDSYQNRLF
YTLKTGETVADLSKSQDINLSTIWSLNKHLYSSESEMMKAAPGQQIILPLKKLPFEYSALPLLG
SAPLVAAGGVAGHTNKLTKMSPDVTKSNMTDDKALNYAAQQAASLGSQLQSRSLNGDYAKDTAL
GIAGNQASSQLQAWLQHYGTAEVNLQSGNNFDGSSLDFLLPFYDSEKMLAFGQVGARYIDSRFT
ANLGAGQRFFLPANMLGYNVFIDQDFSGDNTRLGIGGEYWRDYFKSSVNGYFRMSGWHESYNKK
DYDERPANGFDIRFNGYLPSYPALGAKLIYEQYYGDNVALFNSDKLQSNPGAATVGVNYTPIPL
VTMGIDYRHGTGNENDLLYSMQFRYQFDKSWSQQIEPQYVNELRTLSGSRYDLVQRNNNIILEY
KKQDILSLNIPHDINGTEHSTQKIQLIVKSKYGLDRIVWDDSALRSQGGQIQHSGSQSAQDYQA
ILPAYVQGGSNIYKVTARAYDRNGNSSNNVQLTITVLSNGQVVDQVGVTDFTADKTSAKADNAD
TITYTATVKKNGVAQANVPVSFNIVSGTATLGANSAKTDANGKATVTLKSSTPGQVVVSAKTAE
MTSALNASAVIFFDGATRVPTLTGILVNGQNFATDKGFPKTIFKNATFQLQMDNDVANNTQYEW
SSSFTPNVSVNDQGQVTITYQTYSEVAVTAKSKKFPSYSVSYRFYPNRWIYDGGTSLVSSLEAS
RQCQGSDMSAVLESSRATNGTRAPDGTLWGEWGSLTAYSSDWQSGEYWVKKTSTDFETMNMDTG
ALVQGPAYLAFPLCALAI
Sequences of Lysins (e.g. Listeriolysin O and Derivatives)
SEQ ID NO: 2
Amino acid sequence of Listeriolysin O
MKKIMLVFITLILISLPIAQQTEAKDASAFHKEDLISSMAPPTSPPASPKTPIEKKHADEIDKY
IQGLDYNKNNVLVYHGDAVTNVPPRKGYKDGNEYIVVEKKKKSINQNNADIQVVNAISSLTYPG
ALVKANSELVENQPDVLPVKRDSLTLSIDLPGMTNQDNKIVVKNATKSNVNNAVNTLVERWNEK
YAQAYPNVSAKIDYDDEMAYSESQLIAKFGTAFKAVNNSLNVNFGAISEGKMQEEVISFKQIYY
NVNVNEPTRPSRFFGKAVTKEQLQALGVNAENPPAYISSVAYGRQVYLKLSTNSHSTKVKAAFD
AAVSGKSVSGDVELTNIIKNSSFKAVIYGGSAKDEVQIIDGNLGDLRDILKKGATFNRETPGVP
IAYTTNFLKDNELAVIKNNSEYIETTSKAYTDGKINIDHSGGYVAQFNISWDEINYDPEGNEIV
QHKNWSENNKSKLAHFTSSIYLPGNARNINVYAKECTGLAWEWWRTVIDDRNLPLVKNRNISIW
GTTLYPKYSNSVDNPIE
SEQ ID NO: 6
Amino acid sequence of Listeriolysin O lacking periplasmic secretion signal
MDASAFHKEDLISSMAPPTSPPASPKTPIEKKHADEIDKYIQGLDYNKNNVLVYHGDAVTNVPP
RKGYKDGNEYIVVEKKKKSINQNNADIQVVNAISSLTYPGALVKANSELVENQPDVLPVKRDSL
TLSIDLPGMTNQDNKIVVKNATKSNVNNAVNTLVERWNEKYAQAYPNVSAKIDYDDEMAYSESQ
LIAKFGTAFKAVNNSLNVNFGAISEGKMQEEVISFKQIYYNVNVNEPTRPSRFFGKAVTKEQLQ
ALGVNAENPPAYISSVAYGRQVYLKLSTNSHSTKVKAAFDAAVSGKSVSGDVELTNIIKNSSFK
AVIYGGSAKDEVQIIDGNLGDLRDILKKGATFNRETPGVPIAYTTNFLKDNELAVIKNNSEYIE
TTSKAYTDGKINIDHSGGYVAQFNISWDEINYDPEGNEIVQHKNWSENNKSKLAHFTSSIYLPG
NARNINVYAKECTGLAWEWWRTVIDDRNLPLVKNRNISIWGTTLYPKYSNSVDNPIE
Sequences of Detection Markers
SEQ ID NO: 5
iRFP670 Protein
MARKVDLTSCDREPIHIPGSIQPCGCLLACDAQAVRITRITENAGAFFGRETPRVGELLADYFG
ETEAHALRNALAQSSDPKRPALIFGWRDGLTGRTFDISLHRHDGTSIIEFEPAAAEQADNPLRL
TRQIIARTKELKSLEEMAARVPRYLQAMLGYHRVMLYRFADDGSGMVIGEAKRSDLESFLGQHF
PASLVPQQARLLYLKNAIRVVSDSRGISSRIVPEHDASGAALDLSFAHLRSISPCHLEFLRNMG
VSASMSLSIIIDGTLWGLIICHHYEPRAVPMAQRVAAEMFADFLSLHFTAAHHQR

1. A method for detecting diseased epithelial tissue selected from gastrointestinal tract epithelium and bile duct epithelium, comprising:
(i) administering to the gastrointestinal tract of a subject in need thereof, a genetically engineered microorganism,
wherein the microorganism comprises an exogenous gene encoding a surface protein, wherein the surface protein specifically interacts with one or more cell membrane receptor(s),
wherein the one or more cell membrane receptor(s) are not exposed to the luminal side of epithelial cells of normal gastrointestinal tissue and/or epithelial tissue lining the bile duct, pancreatic duct, or common bile duct, etc.; and
wherein the one or more cell membrane receptor(s) are exposed to the luminal side of diseased epithelial cells of gastrointestinal tissue and/or epithelial tissue lining the bile duct, pancreatic duct, or common bile duct, etc. in the subject suffering from a disease,
wherein the surface protein promotes binding and invasion of the microorganism in the diseased epithelial cells,
wherein the microorganism comprises one or more gene(s) encoding at least one detection marker operably linked to a promoter; and
(ii) detecting the expression of the detection marker in epithelial cells to thereby detect diseased epithelial cells.

2.-3. (canceled)

4. The method of claim 1, wherein the promoter is a mammalian promoter or a microbial promoter, optionally wherein the mammalian promoter directs GI tract epithelial cell-specific expression.

5. (canceled)

7. The method of claim 1, wherein the one or more gene(s) encoding at least one detection marker further comprises an internal ribosome entry site (IRES) and/or an intron.

8. The method of claim 1, wherein the genetically engineered microorganism is administered via oral or rectal route.

9. (canceled)

10. The method of claim 1, wherein the at least one detection marker is selected from a fluorescent protein, a bioluminescent protein, a contrast agent for magnetic resonance imaging (MRI), a Positron Emission Tomography (PET) reporter, an enzyme reporter, a contrast agent for use in computerized tomography (CT), a Single Photon Emission Computed Tomography (SPECT) reporter, a photoacoustic reporter, an X-ray reporter, an ultrasound reporter (e.g. a bacterial gas vesicle), and ion channel reporters (e.g. cAMP activated cation channel), and a combination of any two or more thereof.

11. (canceled)

12. The method of claim 10, wherein the fluorescent protein is a near-infrared fluorescent protein selected from iRFP670, miRFP670, iRFP682, iRFP702, miRFP703, miRFP709, iRFP713 (iRFP), iRFP720 and iSplit.

13.-25. (canceled)

26. The method of claim 1, wherein the detection of the abnormal cells is performed using endoscopy, colonoscopy, MRI, CT scan, PET scan, SPECT, or a combination thereof.

27. The method of claim 1, wherein the surface protein comprises an invasin, intimin, or a fragment thereof.

28.-30. (canceled)

31. The method of claim 1, wherein the microorganism comprises a second exogenous gene encoding a lysin that lyses the endocytotic vacuole.

32.-33. (canceled)

34. The method of claim 1, wherein the microorganism is based on Escherichia coli Nissle 1917 or a derivative thereof.

35.-36. (canceled)

37. The method of claim 31, wherein the gene encoding the surface protein, the second gene encoding the lysin and/or at least one detection marker are integrated at a genomic site.

38.-41. (canceled)

42. The method of claim 1, wherein the gene encoding the surface protein, the second gene encoding the lysin and/or one or more gene(s) encoding at least one detection marker are inserted on a plasmid, which is optionally selected from the plasmid pMUT1, the plasmid pMUT2, and/or a derivative thereof.

43.-45. (canceled)

46. The method of claim 42, wherein the plasmid comprises a selection mechanism, selected from an antibiotic resistance marker, a toxin-antitoxin system, a marker causing complementation of a mutation in an essential gene, a cis acting genetic element and a combination of any two or more thereof.

47.-50. (canceled)

51. The method of claim 44, wherein the essential gene is selected from dapA, dapD, murA, air, dadX, murI, dapE, thyA and a combination of any two or more thereof, optionally wherein the essential genes are a combination of alr and dadX, and optionally wherein the plasmid is selected by complementation of the alr and dadX mutations by a functional alr gene present on the plasmid and/or the second plasmid.

52.-53. (canceled)

54. The method of claim 1, wherein the microorganism harbors at least one nutritional auxotrophic mutation selected from dapA, dapD, dapE, murA, air, dadX, murI, thyA, aroC, ompC, and ompF , optionally wherein the strain harbors a combination of dapA, alr and dadX auxotrophic mutations.

55.-59. (canceled)

60. The method of claim 1, wherein the disease is selected from a precancerous lesion, cancer, ulcerative colitis, Crohn's disease, Barrett's esophagus, irritable bowel syndrome and irritable bowel disease.

61. The method of claim 60, wherein the precancerous lesion comprises:
a polyp selected from sessile polyp, serrated polyp (e.g. hyperplastic polyps, sessile serrated adenomas/polyps, and traditional serrated adenoma), sessile serrated polyp, flat polyp, sub-pedunculated polyp , pedunculated polyp, and a combination thereof:
a biliary intraepithelial neoplasm (BilIN) selected from BilIN-1, BilIN-2, BilIN-3, and cholangiocarcinoma, or
a pancreatic intraepithelial neoplasm (PanIN) selected from PanIN-1, PanIN-2, PanIN-3 and pancreatic ductal adenocarcinoma (PDAC).

62.-65. (canceled)

66. The method of claim 60, wherein the precancerous lesion has a size of less than about 0.1 mm, less than about 0.25 mm, less than about 0.5 mm, less than about 1 mm, less than about 2 mm, less than about 5 mm, less than about 8 mm, less than about 10 mm, less than about 15 mm, less than about 20 mm, less than about 25 mm, or less than about 30 mm.

67.-69. (canceled)

70. A genetically engineered microorganism comprising a gene encoding a surface protein, wherein the surface protein specifically interacts with one or more cell membrane(s) receptor,
wherein the one or more cell membrane receptor(s) are not exposed to the luminal side of epithelial cells of normal gastrointestinal tissue and/or epithelial tissue lining the bile duct, pancreatic duct, or common bile duct, etc.; and
wherein the one or more cell membrane receptor(s) are exposed to the luminal side of epithelial cells of diseased gastrointestinal tissue and/or epithelial tissue lining the bile duct, pancreatic duct, or common bile duct, etc.,
wherein the surface protein promotes binding and invasion of epithelial cells of diseased gastrointestinal tissue and/or epithelial tissue lining the bile duct, pancreatic duct, or common bile duct, etc.,
wherein the microorganism comprises one or more gene(s) encoding at least one detection marker operably linked to a promoter.

71.-121. (canceled)

122. A method of selecting a subject suffering from or suspected to be suffering from a disease for a treatment, the method comprising:
(i) administering to the gastrointestinal tract of the subject the genetically engineered microorganism of claim 70;
(ii) detecting elevated expression of the detection marker compared to surrounding normal epithelial cells; and
(iii) selecting the subject for treatment if expression of the detection marker is observed compared to surrounding normal epithelial cells.

123.-132. (canceled)
